(19) United States
(12) Patent Application Publication
(10) Pub. No.: US 2023/0272442 A1
(43) Pub. Date: Aug. 31, 2023

(54) 5-METHYLFOLATE PRODUCING MICROORGANISM

(71) Applicant: Chifeng Pharmaceutical Co., Ltd., Chifeng (CN)

(72) Inventors: Mingan Shi, Chifeng (CN); Xin Xiong, Chifeng (CN); Jia Sun, Chifeng (CN); Jing Zuo, Chifeng (CN); Yunchong Xia, Chifeng (CN); Zailing Zhu, Chifeng (CN); Zhigang Cai, Chifeng (CN); Guoying Zhang, Chifeng (CN); Marko Blazlc, Domzale (SI); Tina Kogej, Ljubljana (SI); Gregor Kosec, Ljubljana (SI); Mirjan Svageij, Ljubljana (SI); Jaka Horvat, Kranj (SI); Stefan Fujs, Ljubljana (SI)

(21) Appl. No.: 18/015,840

(22) PCT Filed: Jun. 30, 2021
(86) PCT No.: PCT/IB2021/055845

§ 371 Date: Jan. 12, 2023

(30) Foreign Application Priority Data
Jul. 15, 2020 (EP) 20186028.5
Aug. 5, 2020 (EP) 20189690.9

(51) Int. Cl. C12P 17/18; C12N 9/10; C12N 9/06; C12N 9/00; C12N 1/20; C12N 15/52; C12N 15/75

(52) U.S. Cl. CPC C12P 17/182; C12N 9/1007; C12N 9/0028; C12N 9/93; C12N 1/20; C12N 15/52; C12N 15/75; C12Y 201/01014; C12Y 105/0102; C12Y 603/02012; C12Y 603/02017

(57) ABSTRACT

The present invention provides a 5-methylfolate producing microorganism which a) has been modified to have a decreased expression and/or activity of a polypeptide having both dihydrofolate synthase activity and folylpolyglutamate synthetase activity compared to an otherwise identical microorganism (reference microorganism); b) has been (further) modified to express a heterologous polypeptide having only dihydrofolate synthase activity; c) has been (further) modified to have an increased expression level of at least one enzyme (such as at least two, at least three, at least four, at least five, at least six, at least seven or at least eight) enzymes involved in the biosynthesis of a 5-methylfolate compared to an otherwise identical microorganism (reference microorganism); and/or d) has been (further) modified to have a decreased expression and/or activity of a polypeptide having 5-methyltetrahydropteroyltriglutamate-homocysteine S-methyltransferase activity compared to an otherwise identical microorganism (reference microorganism).

### TECHNICAL FIELD

The invention relates to the field of biotechnology engineering, in particular to a 5-methylfolate, such as 5-methyl-tetrahydrofolate (5-methyl-THF), producing microorganism and to the preparation and use thereof. More specifically, the present invention provides a 5-methylfolate producing microorganism, such as a 5-methylfolate producing bacterium, which a) has been modified to have an increased expression level of at least one enzyme (such as at least two, at least three, at least four, at least five, at least six, at least seven or at least eight enzymes) involved in the biosynthesis of a 5-methylfolate compared to an otherwise identical microorganism that does not carry said modification (reference microorganism); b) has been (further) modified to have a decreased expression and/or activity of an endogenous polypeptide having 5-methyltetrahydropteroyltriglutamate-homocysteine S-methyltransferase activity compared to an otherwise identical microorganism that does not carry said modification (reference microorganism); c) has been (further) modified to have a decreased expression and/or activity of an endogenous polypeptide having both dihydrofolate synthase activity and folylpolyglutamate synthetase activity compared to an otherwise identical microorganism that does not carry said modification (reference microorganism); and/or d) has been (further) modified to express a heterologous polypeptide having only dihydrofolate synthase activity.

### BACKGROUND

Folate is a general term for folic acid and a number of its derivatives; they differ in the state of oxidation, one-carbon substitution of the pteridine ring and in the number of γ-linked glutamate residues (shown in FIG. 1). The pteridine moiety of folates can exist in three oxidation states: fully oxidized (folic acid), or as the reduced 7,8-dihydrofolate (DHF), or 5,6,7,8-tetrahydrofolate (THF). THF is the co-enzymatically active form of the vitamin that accepts, transfers, and donates Cl groups, which are attached either at the N5 or N10 position or by bridging these positions. The Cl groups also differ in their oxidation state, with folates existing as derivatives of formate (5-formyl-THF (5-FTHF or folinic acid), 10-formyl-THF, 5,10-methenyl-THF, and 5-forminino-THF), methanol (5-methyl-THF; 5-MTHF) or formaldehyde (5,10-methylene-THF). In addition, most naturally occurring folates exist as γ-linked polyglutamate conjugates.

Folic acid (pteroyl-L-glutamic acid) is a synthetic compound, which does not exist in nature. Folic acid is not active as a coenzyme and has to undergo several metabolic steps within the cell to be converted into the metabolically active THF form. However, folic acid is the commercially most important folate compound, produced industrially by chemical synthesis. Mammals cannot synthesize folates and depend on dietary supplementation to maintain normal levels of folates. Low folate status may be caused by low dietary intake, poor absorption of ingested folate and alteration of folate metabolism due to genetic defects or drug interactions. Most countries have established recommended intakes of folate through folic acid supplements or fortified foods. Folates used in diet supplementation include folic acid, folinic acid (5-FTHF, Leucovorin) or 5-MTHF (Scaglione and Panzavolta 2014). Two salt forms of 5-MTHF are currently produced as supplements. Merck Millipore produces Metafolin®, a calcium salt of 5-MTHF, which is a stable crystalline form of the naturally-occurring predominant form of folate. Gnosis S.p.A. developed and patented a glucosamine salt of (6S)-5-MTHF, brand named Quatrefolic®.

Currently, folic acid is industrially primarily produced through chemical synthesis while, unlike other vitamins, microbial production of folic acid on industrial scale is not exploited due to the low yields of folic acid produced by current bacterial strains (Rossi et al., 2016). Although chemically produced folic acid is not a naturally occurring molecule human beings are able to metabolize it into biological active forms of folates by the action of the enzyme dihydrofolate reductase (DHFR). Several reasons support the replacement of chemical synthesis methods by microbial fermentation for commercial production of folates: first, reduced forms of folic acid can be produced by microorganisms, which can be used by humans more efficiently. Most importantly, a single step fermentation process can in principle be much more efficient and environmentally friendly than a multi-stage chemical process.

Previous studies have been done to elucidate folate/folic acid production in microorganisms. Most of microbial application for the production of folates is limited to the fortification of fermented dairy products and to folate-producing probiotics. The optimization of the culture conditions to improve the synthesis of folates have been also carried out, reaching folic acid yields of about 150 μg/g (Hjortmo et al., 2008; Sybesma et al., 2003b). A few studies have described genetically modified strains either of lactic acid bacteria (Sybesma et al., 2003a), yeasts (Walkey et al., 2015) or filamentous fungus (Serrano-Amatriain et al. 2016), which are able to produce folic acid with titers of up to 6.6 mg/L. Another successfully used approach for microbial production of folates is cultivation of yeast or bacterial strains in the presence of para-aminobenzoic acid (pABA). Total folate content of up to 22 mg/L were measured in supernatants of these cultures.

(6S)-5-methyltetrahydrofolate (L-5-methyltetrahydrofolate or L-5-methyl-THF) is an active form of folic acid (vitamin B9). Folic acid (pteroyl-L-glutamic acid) is a synthetic compound, produced industrially by chemical synthesis and is not found in fresh natural foods. Folic acid is not active as a coenzyme and has to undergo several metabolic steps within the cell to be converted into the metabolically active folate form. On the other hand, 5-methyl tetrahydrofolate is the predominant form of dietary folate and also predominant active form of folate in the human body, which accounts for approximately 98% of folates in human plasma. Intake of L-5-methyl-THF may have several advantages over intake of folic acid, such as reduced potential for masking the haematological symptoms of vitamin B12 deficiency, reduced interference with drugs that targets dihydrofolate reductase and L-5-methyl-THF does not accumulates in human plasma as unmetabolized vitamin.

The industrial technology for chemical synthesis of folic acid is hampered by a huge environmental burden. So far, microbial fermentation processes to produce folic acid or any of the natural forms of folates, such as 5-methylfolate, have not been competitive due to very low production titers/yields of folates.

Therefore, there is an urgent need to develop new genetically engineering microorganisms for enhancing the production capacity of 5-methylfolate, such as 5-methyl-tetrahydrofolate or a precursor or an intermediate thereof.

### SUMMARY OF THE INVENTION

The object of the present invention is to provide genetically engineered microorganisms for enhancing the production capacity of 5-methylfolate (such as 5-methyl-tetrahydrofolate) or a precursor or an intermediate thereof. This object is solved by the present inventors.

The present invention may be summarized by way of the following items.
1. A genetically engineered microorganism, such as bacterium.
2. The genetically engineered microorganism according to item 1, which has been modified to have a decreased expression and/or activity of an endogenous polypeptide having both dihydrofolate synthase activity and folylpolyglutamate synthetase activity compared to an otherwise identical microorganism that does not carry said modification (reference microorganism).
3. The genetically engineered microorganism according to item 2, which has been modified to have a decreased expression level of the endogenous gene encoding said polypeptide having both dihydrofolate synthase activity and folylpolyglutamate synthetase activity compared to an otherwise identical microorganism that does not carry said modification (reference microorganism).
4. The genetically engineered microorganism according to item 3, wherein the expression level of the endogenous gene is decreased by at least 50%, such as by at least 60%, at least 65%, at least 70%, at least 75%, at least 80%, at least 85%, at least 90%, at least 95% or at least 100% compared to the otherwise identical microorganism.
5. The genetically engineered microorganism according to item 3 or 4, wherein the endogenous gene encoding said polypeptide having both dihydrofolate synthase activity and folylpolyglutamate synthetase activity has been inactivated.
6. The genetically engineered microorganism according to item 5, wherein the endogenous gene encoding said polypeptide having both dihydrofolate synthase activity and folylpolyglutamate synthetase activity has been inactivated by deletion of part of or the entire gene sequence.
7. The genetically engineered microorganism according to item 4 or 5, wherein the endogenous gene encoding said polypeptide having both dihydrofolate synthase activity and folylpolyglutamate synthetase activity has been inactivated by introducing or expressing in the microorganism a rare-cutting endonuclease able to selectively inactivating by DNA cleavage the endogenous gene encoding said polypeptide.
8. The genetically engineered microorganism according to item 7, wherein said rare-cutting endonuclease is a transcription activator-like effector (TALE) nuclease, meganuclease, zing-finger nuclease (ZFN), or RNA-guided endonuclease.
9. The genetically engineered microorganism according to item 8, wherein the RNA-guided endonuclease is a catalytically inactive Cas9 protein.
10. The genetically engineered microorganism according to item 13, which comprises (e.g., expresses) a single guide RNA (sgRNA) specifically hybridizing (e.g. binding) under cellular conditions with the genomic DNA encoding said polypeptide.
11. The genetically engineered microorganism according to item 2, wherein the expression of said endogenous polypeptide having both dihydrofolate synthase activity and folylpolyglutamate synthetase activity is decreased (e.g., inhibited) by transcriptional and/or translational repression of the endogenous gene encoding said polypeptide.
12. The genetically engineered microorganism according to item 2, wherein the expression of said endogenous polypeptide having both dihydrofolate synthase activity and folylpolyglutamate synthetase activity is decreased (e.g. inhibited) by introducing or expressing in the microorganism an inhibitory nucleic acid molecule that specifically hybridizes (e.g. binds) under cellular conditions with cellular mRNA and/or genomic DNA encoding said polypeptide.
13. The genetically engineered microorganism according to item 12, wherein the inhibitory nucleic acid molecule is an antisense oligonucleotide, ribozyme or interfering RNA (RNAi) molecule.
14. The genetically engineered microorganism according to item 13, wherein the interfering RNA molecule is a micro RNA (miRNA), small interfering RNA (siRNA) or short hairpin RNA (shRNA).
15. The genetically engineered microorganism according to item 2 or 3, which has been modified to have a decreased activity of an endogenous polypeptide having both dihydrofolate synthase activity and folylpolyglutamate synthetase activity compared to an otherwise identical microorganism that does not carry said modification (reference microorganism).
16. The genetically engineered microorganism according to item 15, wherein the activity of said polypeptide is decreased by at least one active-site mutation resulting in the reduction or loss of activity.
17. The genetically engineered microorganism according to item 16, wherein the at least one active-site mutation is at a position which corresponds to any one of positions 51-54, 75, 114-117, 145, 152-154, 172, 263, 302 and 315 in the amino acid sequence set forth in SEQ ID NO: 11.
18. The genetically engineered microorganism according to item 16 or 17, wherein the at least one active-site mutation is a non-conservative amino acid substitution.
19. The genetically engineered microorganism according to any one of items 2 to 18, wherein the endogenous gene encoding said polypeptide having both dihydrofolate synthase activity and folylpolyglutamate synthetase activity is the gene folC.
20. The genetically engineered microorganism according to any one of items 2 to 18, wherein the endogenous gene encoding said polypeptide having both dihydrofolate synthase activity and folylpolyglutamate synthetase activity is the endogenous gene folC.
21. The genetically engineered microorganism according to any one of items 2 to 20, wherein the endogenous gene encoding said polypeptide having both dihydrofolate synthase activity and folylpolyglutamate synthetase activity comprises a nucleic acid sequence having at least 70%, such as at least 80%, at least 85%, at least 90%, at least 93%, at least 95%, at least 98% or at least 99%, sequence identity with the nucleic acid sequence set forth in SEQ ID NO: 5
22. The genetically engineered microorganism according to any one of items 2 to 21, wherein the polypeptide having both dihydrofolate synthase activity and folylpolyglutamate synthetase activity encoded by the endogenous gene comprises an amino acid which has at least 70%, such as at least 80%, at least 85%, at least 90%, at least 93%, at least 95%, at least 98% or at least 99%, sequence identity with the amino acid sequence set forth in SEQ ID NO: 11
23. The genetically engineered microorganism according to any one of items 1 to 22, which has been (further) modified to express a heterologous polypeptide having only dihydrofolate synthase activity.
24. The genetically engineered microorganism according to item 23, wherein the heterologous polypeptide having only dihydrofolate synthase activity is derived from a bacterium or fungus, preferably selected from Lactobacillus reuteri and Ashbya gossypii.
24. The genetically engineered microorganism according to item 23 or 24, wherein the heterologous polypeptide having only dihydrofolate synthase activity comprises an amino acid sequence having at least 70%, such as at least 80%, at least 85%, at least 90%, at least 93%, at least 95%, at least 98% or at least 99%, sequence identity with SEQ ID NO: 22 or 23.
25. The genetically engineered microorganism according to any one of items 1 to 24, which has been (further) modified to have a significantly improved production capacity of a 5-methylfolate (such as 5-methyl-tetrahydrofolate) or a precursor or an intermediate thereof compared to an otherwise identical microorganism that does not carry said modification (reference microorganism).
26. The genetically engineered microorganism according to item 25, wherein the production capacity of a 5-methylfolate (such as 5-methyl-tetrahydrofolate) or a precursor or an intermediate thereof is increased by at least 50%, such as at least 100%, at least 200%, at least 500%, at least 1000%, at least 2000%, at least 5000%, at least 10000%, at least 20000% or at least 50000%, compared to an otherwise identical microorganism that does not carry said modification (reference microorganism).
27. The genetically engineered microorganism according to any one of items 1 to 26, which has been (further) modified to have an increased expression level of at least one gene (such as at least two, at least three, at least four, at least five, at least six, at least seven or at least eight genes) encoding an enzyme involved in the biosynthesis of a 5-methylfolate (such as 5-methyl-tetrahydrofolate) compared to an otherwise identical microorganism that does not carry said modification (reference microorganism).
28. The genetically engineered microorganism according to any one of items 1 to 27, which has been (further) modified to have an increased expression level of at least two genes (such as at least three, at least four, at least five, at least six, at least seven or at least eight genes) encoding enzymes involved in the biosynthesis of a 5-methylfolate (such as 5-methyl-tetrahydrofolate) compared to an otherwise identical microorganism that does not carry said modification (reference microorganism).
29. The genetically engineered microorganism according to any one of items 1 to 28, which has been (further) modified to have an increased expression level of at least three genes (such as at least four, at least five, at least six, at least seven or at least eight genes) encoding enzymes involved in the biosynthesis of a 5-methylfolate compared to an otherwise identical microorganism that does not carry said modification (reference microorganism).
30. The genetically engineered microorganism according to any one of items 1 to 29, which has been (further) modified to have an increased expression level of at least four genes (such as at least five, at least six, at least seven or at least eight genes) encoding enzymes involved in the biosynthesis of a 5-methylfolate (such as 5-methyl-tetrahydrofolate) compared to an otherwise identical microorganism that does not carry said modification (reference microorganism).
31. The genetically engineered microorganism according to any one of items 1 to 30, which has been (further) modified to have an increased expression level of at least five genes (such as at least six genes; at least seven genes or at least eight genes) encoding enzymes involved in the biosynthesis of a 5-methylfolate (such as 5-methyl-tetrahydrofolate) compared to an otherwise identical microorganism that does not carry said modification (reference microorganism).
32. The genetically engineered microorganism according to any one of items 1 to 31, which has been (further) modified to have an increased expression level of at least six genes (such as at least seven genes or at least eight genes) encoding enzymes involved in the biosynthesis of a 5-methylfolate (such as 5-methyl-tetrahydrofolate) compared to an otherwise identical microorganism that does not carry said modification (reference microorganism).
33. The genetically engineered microorganism according to any one of items 1 to 32, which has been (further) modified to have an increased expression level of at least seven genes (such as at least eight genes) encoding enzymes involved in the biosynthesis of a 5-methylfolate (such as 5-methyl-tetrahydrofolate) compared to an otherwise identical microorganism that does not carry said modification (reference microorganism).
34. The genetically engineered microorganism according to any one of items 27 to 33, wherein the at least one gene encoding an enzyme involved in the biosynthesis of a 5-methylfolate (such as 5-methyl-tetrahydrofolate) is selected from the group consisting of folE/mtrA, folB, folK, folP/sul, folA/dfrA, glyA, purU, yitJ and metF.
35. The genetically engineered microorganism according to any one of items 27 to 34, wherein the enzyme involved in the biosynthesis of a 5-methylfolate, such as 5-methyl-tetrahydrofolate, is selected from the group consisting of: a polypeptide having GTP cyclohydrolase activity, a polypeptide having 7,8-dihydroneopterin aldolase activity, a polypeptide having 2-amino-4-hydroxy-6-hydroxymethyldihydropteridine pyrophosphokinase activity, a polypeptide having dihydropteroate synthase activity, a polypeptide having dihydrofolate reductase activity, a polypeptide having serine hydroxymethyltransferase activity, a polypeptide having formyltetrahydrofolate deformylase activity, and a polypeptide having 5,10-methylenetetrahydrofolate reductase activity.
36. The genetically engineered microorganism according to any one of items 1 to 34, which has been (further) modified to have an increased expression level of a gene encoding a polypeptide having GTP cyclohydrolase activity compared to an otherwise identical microorganism that does not carry said modification (reference microorganism).
37. The genetically engineered microorganism according to any one of items 1 to 36, which has been (further) modified to have an increased expression level of a gene encoding a polypeptide having 7,8-dihydroneopterin aldolase activity compared to an otherwise identical microorganism that does not carry said modification (reference microorganism).
38. The genetically engineered microorganism according to any one of items 1 to 37, which has been (further) modified to have an increased expression level of a gene encoding a polypeptide having 2-amino-4-hydroxy-6-hydroxymethyldihydropteridine pyrophosphokinase activity compared to an otherwise identical microorganism that does not carry said modification (reference microorganism).
39. The genetically engineered microorganism according to any one of items 1 to 38, which has been (further) modified to have an increased expression level of a gene encoding a polypeptide having dihydropteroate synthase activity compared to an otherwise identical microorganism that does not carry said modification (reference microorganism).
40. The genetically engineered microorganism according to any one of items 1 to 39, which has been (further) modified to have an increased expression level of a gene encoding a polypeptide having dihydrofolate reductase activity compared to an otherwise identical microorganism that does not carry said modification (reference microorganism).
41. The genetically engineered microorganism according to any one of items 1 to 40, which has been (further) modified to have an increased expression level of a gene encoding a polypeptide having serine hydroxymethyltransferase activity compared to an otherwise identical microorganism that does not carry said modification (reference microorganism).
42. The genetically engineered microorganism according to any one of items 1 to 41, which has been (further) modified to have an increased expression level of a gene encoding a polypeptide having formyltetrahydrofolate deformylase activity compared to an otherwise identical microorganism that does not carry said modification (reference microorganism).
43. The genetically engineered microorganism according to any one of items 1 to 42, which has been (further) modified to have an increased expression level of a gene encoding a polypeptide having 5,10-methylenetetrahydrofolate reductase activity compared to an otherwise identical microorganism that does not carry said modification (reference microorganism).
44. The genetically engineered microorganism according to any one of items 27 to 43, wherein the at least one gene encoding an enzyme involved in the biosynthesis of a 5-methylfolate (such as 5-methyl-tetrahydrofolate) is heterologous to the genetically engineered microorganism.
45. The genetically engineered microorganism according to any one of items 27 to 44, wherein the at least one gene encoding an enzyme involved in the biosynthesis of a 5-methylfolate is derived from a bacterium or fungus, preferably selected from the genus Bacillus, Escherichia, Lactococcus, Shewanella, Vibrio and Ashbya.
46. The genetically engineered microorganism according to any one of items 27 to 45, wherein the at least one gene encoding an enzyme involved in the biosynthesis of a 5-methylfolate (such as 5-methyl-tetrahydrofolate) is derived from a bacterium or fungus selected from Bacillus subtilis, Lactobacillus lactis, Escherichia coli, Shewanella violacea, Vibrio natriegens or Ashbya gossypii.
47. The genetically engineered microorganism according to any one of items 27 to 46, wherein the expression level of at least one gene (such as at least two, at least three, at least four, at least five, at least six, at least seven or at least eight genes) encoding an enzyme involved in the biosynthesis of a 5-methylfolate (such as 5-methyl-tetrahydrofolate) is increased by at least 50%, at least 100, at least 200%, at least 500%, at least 1000%, at least 2000%, at least 5000%, at least 10000%, at least 20000% or at least 50000%, compared to the otherwise identical microorganism (reference microorganism).
48. The genetically engineered microorganism according to any one of items 1 to 47, which has been further modified to have an increased expression level of at least one enzyme (such as at least two, at least three, at least four, at least five, at least six, at least seven or at least eight) enzymes involved in the biosynthesis of a 5-methylfolate (such as 5-methyl-tetrahydrofolate) compared to an otherwise identical microorganism that does not carry said modification (reference microorganism).
49. The genetically engineered microorganism according to any one of items 1 to 47, which has been further modified to have an increased expression level of at least two (such as at least three, at least four, at least five, at least six, at least seven or at least eight) enzymes involved in the biosynthesis of a 5-methylfolate (such as 5-methyl-tetrahydrofolate), compared to an otherwise identical microorganism that does not carry said modification (reference microorganism).
50. The genetically engineered microorganism according to any one of items 1 to 47, which has been further modified to have an increased expression level of at least three (such as at least four, at least five, at least six, at least seven or at least eight) enzymes involved in the biosynthesis of a 5-methylfolate (such as 5-methyl-tetrahydrofolate) compared to an otherwise identical microorganism that does not carry said modification (reference microorganism).
51. The genetically engineered microorganism according to any one of items 1 to 47, which has been further modified to have an increased expression level of at least four (such as at least five, at least six, at least seven or at least eight) enzymes involved in the biosynthesis of a 5-methylfolate (such as 5-methyl-tetrahydrofolate) compared to an otherwise identical microorganism that does not carry said modification (reference microorganism).
52. The genetically engineered microorganism according to any one of items 1 to 47, which has been further modified to have an increased expression level of at least five (such as at least six, at least seven or at least eight) enzymes involved in the biosynthesis of a 5-methylfolate (such as 5-methyl-tetrahydrofolate) compared to an otherwise identical microorganism that does not carry said modification (reference microorganism).
53. The genetically engineered microorganism according to any one of items 1 to 47, which has been further modified to have an increased expression level of at least six (such as at least seven or at least eight) enzymes involved in the biosynthesis of a 5-methylfolate (such as 5-methyl-tetrahydrofolate) compared to an otherwise identical microorganism that does not carry said modification (reference microorganism).
54. The genetically engineered microorganism according to any one of items 1 to 47, which has been (further) modified to have an increased expression level of at least seven (such as at least eight) enzymes involved in the biosynthesis of a 5-methylfolate (such as 5-methyl-tetrahydrofolate) compared to an otherwise identical microorganism that does not carry said modification (reference microorganism).
55. The genetically engineered microorganism according to any one of items 48 to 54, wherein said at least one enzyme involved in the biosynthesis of 5-methylfolate (such as 5-methyl-tetrahydrofolate) is selected from the group consisting of: a polypeptide having GTP cyclohydrolase activity, a polypeptide having 7,8-dihydroneopterin aldolase activity, a polypeptide having 2-amino-4-hydroxy-6-hydroxymethyldihydropteridine pyrophosphokinase activity, a polypeptide having dihydropteroate synthase activity, a polypeptide having dihydrofolate reductase activity, a polypeptide having serine hydroxymethyltransferase activity, a polypeptide having formyltetrahydrofolate deformylase activity, and a polypeptide having 5,10-methylenetetrahydrofolate reductase activity.
56. The genetically engineered microorganism according to any one of items 1 to 55, which has been (further) modified to have an increased expression level of a polypeptide having GTP cyclohydrolase activity compared to an otherwise identical microorganism that does not carry said modification (reference microorganism).
57. The genetically engineered microorganism according to any one of items 1 to 56, which has been (further) modified to have an increased expression level of a polypeptide having 7,8-dihydroneopterin aldolase activity compared to an otherwise identical microorganism that does not carry said modification (reference microorganism).
58. The genetically engineered microorganism according to any one of items 1 to 57, which has been (further) modified to have an increased expression level of a polypeptide having 2-amino-4-hydroxy-6-hydroxymethyldihydropteridine pyrophosphokinase activity compared to an otherwise identical microorganism that does not carry said modification (reference microorganism).
59. The genetically engineered microorganism according to any one of items 1 to 58, which has been (further) modified to have an increased expression level of a polypeptide having dihydropteroate synthase activity compared to an otherwise identical microorganism that does not carry said modification (reference microorganism).
60. The genetically engineered microorganism according to any one of items 1 to 59, which has been (further) modified to have an increased expression level of a polypeptide having dihydrofolate reductase activity compared to an otherwise identical microorganism that does not carry said modification (reference microorganism).
61. The genetically engineered microorganism according to any one of items 1 to 60, which has been (further) modified to have an increased expression level of a polypeptide having serine hydroxymethyltransferase activity compared to an otherwise identical microorganism that does not carry said modification (reference microorganism).
62. The genetically engineered microorganism according to any one of items 1 to 61, which has been (further) modified to have an increased expression level of a polypeptide having formyltetrahydrofolate deformylase activity compared to an otherwise identical microorganism that does not carry said modification (reference microorganism).
63. The genetically engineered microorganism according to any one of items 1 to 62, which has been (further) modified to have an increased expression level of a polypeptide having 5,10-methylenetetrahydrofolate reductase activity compared to an otherwise identical microorganism that does not carry said modification (reference microorganism).
64. The genetically engineered microorganism according to any one of items 48 to 63, wherein the at least one enzyme involved in the biosynthesis of a 5-methylfolate (such as 5-methyl-tetrahydrofolate), respectively the recited polypeptide is heterologous to the genetically engineered microorganism.
65. The genetically engineered microorganism according to any one of items 48 to 64, wherein the at least one enzyme involved in the biosynthesis of a 5-methylfolate, respectively the recited polypeptide is derived from a bacterium or fungus, preferably selected from the genus Bacillus, Escherichia, Lactococcus, Shewanella, Vibrio and Ashbya.
66. The genetically engineered microorganism according to any one of items 48 to 65, wherein the at least one enzyme involved in the biosynthesis of a 5-methylfolate (such as 5-methyl-tetrahydrofolate), respectively the recited polypeptide is derived from a bacterium or fungus selected from Bacillus subtilis, Lactobacillus lactis, Escherichia coli, Shewanella violacea, Vibrio natriegens or Ashbya gossypii.
67. The genetically engineered microorganism according to any one of items 35, 36, 55 and 56, wherein the polypeptide having GTP cyclohydrolase activity comprises an amino acid sequence having at least 70%, such as at least 80%, at least 85%, at least 90%, at least 93%, at least 95%, at least 98% or at least 99%, sequence identity with SEQ ID NO: 7.
68. The genetically engineered microorganism according to any one of items 35, 37, 55 and 57, wherein the polypeptide having 7,8-dihydroneopterin aldolase activity comprises an amino acid sequence having at least 70%, such as at least 80%, at least 85%, at least 90%, at least 93%, at least 95%, at least 98% or at least 99%, sequence identity with SEQ ID NO: 8.
69. The genetically engineered microorganism according to any one of items 35, 38, 55 and 58, wherein the polypeptide having 2-amino-4-hydroxy-6-hydroxymethyl-dihydropteridine pyrophosphokinase activity comprises an amino acid sequence having at least 70%, such as at least 80%, at least 85%, at least 90%, at least 95%, at least 98% or at least 99%, sequence identity with SEQ ID NO: 9.
70. The genetically engineered microorganism according to any one of items 35, 39, 55 or 59, wherein the polypeptide having dihydropteroate synthase activity comprises an amino acid sequence having at least 70%, such as at least 80%, at least 85%, at least 90%, at least 93%, at least 95%, at least 98% or at least 99%, sequence identity with SEQ ID NO: 10.
71. The genetically engineered microorganism according to any one of items 35, 40, 55 and 60, wherein the polypeptide having dihydrofolate reductase activity comprises an amino acid sequence having at least 70%, such as at least 80%, at least 85%, at least 90%, at least 93%, at least 95%, at least 98% or at least 99%, sequence identity with SEQ ID NO: 12.
72. The genetically engineered microorganism according to any one of items 35, 41, 55 and 61, wherein the polypeptide having serine hydroxymethyltransferase activity comprises an amino acid sequence having at least 70%, such as at least 80%, at least 85%, at least 90%, at least 93%, at least 95%, at least 98% or at least 99%, sequence identity with SEQ ID NO: 79.
73. The genetically engineered microorganism according to any one of items 35, 42, 55 and 62, wherein the polypeptide having formyltetrahydrofolate deformylase activity comprises an amino acid sequence having at least 70%, such as at least 80%, at least 85%, at least 90%, at least 95%, at least 98% or at least 99%, sequence identity with SEQ ID NO: 81.
74. The genetically engineered microorganism according to any one of items 35, 43, 55 and 63, wherein the polypeptide having 5,10-methylenetetrahydrofolate reductase activity comprises an amino acid sequence having at least 70%, such as at least 80%, at least 85%, at least 90%, at least 93%, at least 95%, at least 98% or at least 99%, sequence identity with SEQ ID NO: 83.
75. The genetically engineered microorganism according to any one of items 35, 43, 55 and 64, wherein the polypeptide having 5,10-methylenetetrahydrofolate reductase activity comprises an amino acid sequence having at least 70%, such as at least 80%, at least 85%, at least 90%, at least 93%, at least 95%, at least 98% or at least 99%, sequence identity with SEQ ID NO: 84.
76. The genetically engineered microorganism according to any one of items 1 to 75, which has been (further) modified to have a decreased expression and/or activity of an endogenous polypeptide having 5-methyltetrahydropteroyltriglutamate-homocysteine S-methyltransferase activity compared to an otherwise identical microorganism that does not carry said modification (reference microorganism).
77. The genetically engineered microorganism according to item 76, which has been modified to have a decreased expression level of an endogenous gene encoding said polypeptide having 5-methyltetrahydropteroyltriglutamate-homocysteine S-methyltransferase activity compared to an otherwise identical microorganism that does not carry said modification (reference microorganism).
78. The genetically engineered microorganism according to item 77, wherein the expression level of the endogenous gene is decreased by at least 50%, such as by at least 60%, at least 65%, at least 70%, at least 75%, at least 80%, at least 85%, at least 90%, at least 95% or at least 100% compared to the otherwise identical microorganism.
79. The genetically engineered microorganism according to item 76 or 77, which comprises at least one mutation in the regulatory region of the endogenous gene encoding said polypeptide having 5-methyltetrahydropteroyltriglutamate-homocysteine S-methyltransferase activity, resulting in the decreased expression level.
80. The genetically engineered microorganism according to item 76 or 77, wherein the endogenous gene encoding said polypeptide having 5-methyltetrahydropteroyltriglutamate-homocysteine S-methyltransferase activity has been inactivated.
81. The genetically engineered microorganism according to item 76 or 77, wherein the endogenous gene encoding said polypeptide having 5-methyltetrahydropteroyltriglutamate-homocysteine S-methyltransferase activity has been inactivated by deletion of part of or the entire gene sequence.
82. The genetically engineered microorganism according to item 76, wherein the endogenous gene encoding said polypeptide having 5-methyltetrahydropteroyltriglutamate-homocysteine S-methyltransferase activity has been inactivated by introducing or expressing in the microorganism a rare-cutting endonuclease able to selectively inactivating by DNA cleavage the endogenous gene encoding said polypeptide.
83. The genetically engineered microorganism according to item 82, wherein said rare-cutting endonuclease is a transcription activator-like effector (TALE) nuclease, meganuclease, zing-finger nuclease (ZFN), or RNA-guided endonuclease.
84. The genetically engineered microorganism according to item 83, wherein the RNA-guided endonuclease is a catalytically inactive Cas9 protein.
85. The genetically engineered microorganism according to item 84, which comprises (e.g., expresses) a single guide RNA (sgRNA) specifically hybridizing (e.g. binding) under cellular conditions with the genomic DNA encoding said polypeptide.
86. The genetically engineered microorganism according to item 76, wherein the expression of said endogenous polypeptide having 5-methyltetrahydropteroyltriglutamate-homocysteine S-methyltransferase activity is decreased (e.g., inhibited) by transcriptional and/or translational repression of the endogenous gene encoding said polypeptide.
87. The genetically engineered microorganism according to item 76, wherein the expression of said endogenous polypeptide having 5-methyltetrahydropteroyltriglutamate-homocysteine S-methyltransferase activity is decreased (e.g., inhibited) by introducing or expressing in the microorganism an inhibitory nucleic acid molecule that specifically hybridizes (e.g. binds) under cellular conditions with cellular mRNA and/or genomic DNA encoding said polypeptide.
88. The genetically engineered microorganism according to item 87, wherein the inhibitory nucleic acid molecule is an antisense oligonucleotide, ribozyme or interfering RNA (RNAi) molecule.
89. The genetically engineered microorganism according to item 88, wherein the interfering RNA molecule is a micro RNA (miRNA), small interfering RNA (siRNA) or short hairpin RNA (shRNA).
90. The genetically engineered microorganism according to item 76 or 77, which has been modified to have a decreased activity of an endogenous polypeptide having 5-methyltetrahydropteroyltriglutamate-homocysteine S-methyltransferase activity compared to an otherwise identical microorganism that does not carry said modification (reference microorganism).
91. The genetically engineered microorganism according to item 90, wherein the activity of said polypeptide is decreased by at least one active-site mutation resulting in the reduction or loss of activity.
92. The genetically engineered microorganism according to item 91, wherein the at least one active-site mutation is at a position which corresponds to any one of positions 18, 21, 112, 117, 119, 435-437, 488, 494, 519-521, 565, 601, 603, 605, 645, 647, 669, 730 and 731 in the amino acid sequence set forth in SEQ ID NO: 100.
92. The genetically engineered microorganism according to item 16 or 17, wherein the at least one active-site mutation is a non-conservative amino acid substitution.
93. The genetically engineered microorganism according to any one of items 76 to 92, wherein the endogenous gene encoding said polypeptide having 5-methyltetrahydropteroyltriglutamate-homocysteine S-methyltransferase activity is the gene metE.
94. The genetically engineered microorganism according to any one of items 76 to 93, wherein the endogenous gene encoding said polypeptide having 5-methyltetrahydropteroyltriglutamate-homocysteine S-methyltransferase activity comprises a nucleic acid sequence having at least 70%, such as at least 80%, at least 85%, at least 90%, at least 93%, at least 95%, at least 98% or at least 99%, sequence identity with the nucleic acid sequence set forth in SEQ ID NO: 101.
95. The genetically engineered microorganism according to any one of items 76 to 94, wherein the polypeptide having 5-methyltetrahydropteroyltriglutamate-homocysteine S-methyltransferase activity encoded by the endogenous gene comprises an amino acid sequence which has at least 70%, such as at least 80, at least 85%, at least 90%, at least 93%, at least 95%, at least 98% or at least 99%, sequence identity with the amino acid sequence set forth in SEQ ID NO: 100.
96. The genetically engineered microorganism according to any one of items 1 to 95, which is a bacterium.
97. The genetically engineered microorganism according to any one of items 1 to 96, which is a bacterium of the family Bacillaceae.
98. The genetically engineered microorganism according to any one of items 1 to 97, which is a bacterium of the genus Bacillus.
99. The genetically engineered microorganism according to any one of items 1 to 98, which is a bacterium of the species Bacillus subtilis.
100. A method for preparing a folate, precursor or intermediate thereof, comprising i) cultivating a genetically engineered microorganism according to any one of items 1 to 99 in a culture medium under suitable culture conditions to obtain a fermentation product containing said folate, precursor or intermediate thereof; and ii) optionally, separating and/or purifying said folate, precursor or intermediate thereof.
101. The method according to item 100, wherein step i) is carried out at a culture temperature in a range from 32 to 42° C., preferably in a range from 34 to 39° C., more preferably in a range from 36 to 39° C., such as at about 37° C.
102. The method according to item 100 or 101, wherein step i) is carried out for a period in the range from 10 to 70 h, preferably in a range from 24 to 60 h, more preferably in a range from 36 to 50 h.
103. The method according to any one of items 100 to 102, wherein step i) is carried out at a pH in the range of 6 to 8, preferably in a range of 6.5 to 7.5, more preferably in a range from 6.8 to 7.2.
104. The method according to any one of items 100 to 102, wherein the folate is a compound of Formula I:

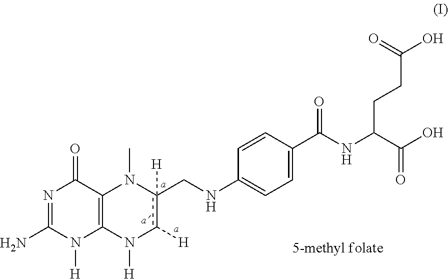

optionally in form of one of the stereoisomers, preferably enantiomers or diastereomers, in form of a racemate or in form of a mixture of at least two of the stereoisomers, preferably enantiomers and/or diastereomers, in any mixing ratio.
105. The method according to any one of items 100 to 104, wherein the folate is a compound of Formula II:

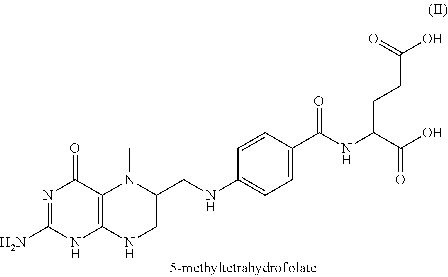 [SMILES: CN1c2c([nH]c(N)nc2=O)NCC1CNc1ccc(C(=O)NC(CCC(=O)O)C(=O)O)cc1]

optionally in form of one of the stereoisomers, preferably enantiomers or diastereomers, in form of a racemate or in form of a mixture of at least two of the stereoisomers, preferably enantiomers and/or diastereomers, in any mixing ratio.
106. The method according to any one of items 100 to 105, wherein the folate is a compound of Formula IIa:

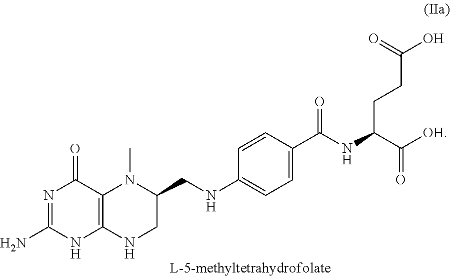 [SMILES: CN1c2c([nH]c(N)nc2=O)NC[C@@H]1CNc1ccc(C(=O)N[C@@H](CCC(=O)O)C(=O)O)cc1]

107. The method according to any one of items 100 to 104, wherein the folate is a compound of Formula III:

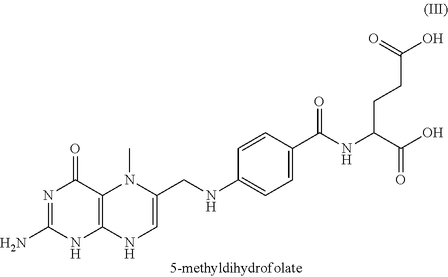 [SMILES: CN1C(CNc2ccc(C(=O)NC(CCC(=O)O)C(=O)O)cc2)=CNc2[nH]c(N)nc(=O)c21]

optionally in form of one of the stereoisomers, preferably enantiomers or diastereomers, in form of a racemate or in form of a mixture of at least two of the stereoisomers, preferably enantiomers and/or diastereomers, in any mixing ratio.
108. The method according to any one of items 100 to 107, further comprising the step of adding para-aminobenzoic acid (PABA) during the cultivation step (i).
109. The method according to item 108, wherein the para-aminobenzoic acid (PABA) is selected from the group consisting of: potassium para-aminobenzoate, sodium para-aminobenzoate, methyl para-aminobenzoate, ethyl para-aminobenzoate, butyl para-aminobenzoate, or a combination thereof.
110. The method according to any one of items 100 to 109, further comprising subjecting the product obtained in the steps (i) or (ii) to acidic or alkaline conditions to further obtain a derivative compound.
111. A method of preparing a genetically engineered microorganism, comprising any one of the steps (a) to (d) below:
(a) increased the expression level of at least one (such as at least two, at least three, at least four, at least five, at least six, at least seven or at least eight) enzyme involved in the biosynthesis of a 5-methylfolate (such as 5-methyl-tetrahydrofolate) compared to an otherwise identical microorganism (reference microorganism);
(b) decreasing the expression and/or activity of an endogenous polypeptide having 5-methyltetrahydropteroyltriglutamate-homocysteine S-methyltransferase activity compared to an otherwise identical microorganism that does not carry said modification (reference microorganism);
(c) decreasing the expression and/or activity of an endogenous polypeptide having both dihydrofolate synthase activity and folylpolyglutamate synthetase activity compared to an otherwise identical microorganism that does not carry said modification (reference microorganism); and/or
(d) expressing a heterologous polypeptide having only dihydrofolate synthase activity.
112. The method according to item 111, comprising any one of the steps (a) to (b) below:
(a) increased the expression level of at least one enzyme involved in the biosynthesis of a 5-methylfolate compared to an otherwise identical microorganism (reference microorganism); and
(b) decreasing the expression and/or activity of an endogenous polypeptide having 5-methyltetrahydropteroyltriglutamate-homocysteine S-methyltransferase activity compared to an otherwise identical microorganism that does not carry said modification (reference microorganism).
113. The method according to claim 112, further comprising the steps (c) and (d) below:
(c) decreasing the expression and/or activity of an endogenous polypeptide having both dihydrofolate synthase activity and folylpolyglutamate synthetase activity compared to an otherwise identical microorganism that does not carry said modification (reference microorganism); and
(d) expressing a heterologous polypeptide having only dihydrofolate synthase activity.
114. The method according to any one of items 111 to 113, comprising the steps of aa) introducing into said microorganism at least one exogenous nucleic acid molecule comprising a nucleic acid sequence encoding an enzyme involved in the biosynthesis of a 5-methylfolate (such as 5-methyl-tetrahydrofolate); bb) inactivating, such as by deleting part of or the entire gene sequence, the endogenous gene encoding said polypeptide having 5-methyltetrahydropteroyltriglutamate-homocysteine S-methyltransferase activity or introducing at least one mutation in the regulatory region of said endogenous gene, which results in the decrease expression level; cc) inactivating, such as by deleting part of or the entire gene sequence, the endogenous gene encoding said polypeptide having both dihydrofolate synthase activity and folylpolyglutamate synthetase activity in said microorganism; and/or dd) introducing into said microorganism an exogenous nucleic acid molecule comprising a nucleic acid sequence encoding a heterologous polypeptide having only dihydrofolate synthase activity.

It should be understood that, within the scope of the present invention, each technical feature of the present invention described above and in the following may be combined with each other to form a preferred technical solution, which is not listed here explicitly due to space limitations.

### DESCRIPTION OF FIGURES

FIG. 1 shows the core structure of folates. In natural folates, the pterin ring exists in tetrahydro form (as shown) or in 7,8-dihydro form. The ring is fully oxidized in chemically produced folic acid. Folates usually have a γ-linked polyglutamyl tail of up to about eight residues attached to the first glutamate. One-carbon unit (formyl, methyl, etc.) can be coupled to the N5 and/or N10 positions resulting in synthesis of 5-formyl folates, 10-formyl folates or 5-methyl folates.

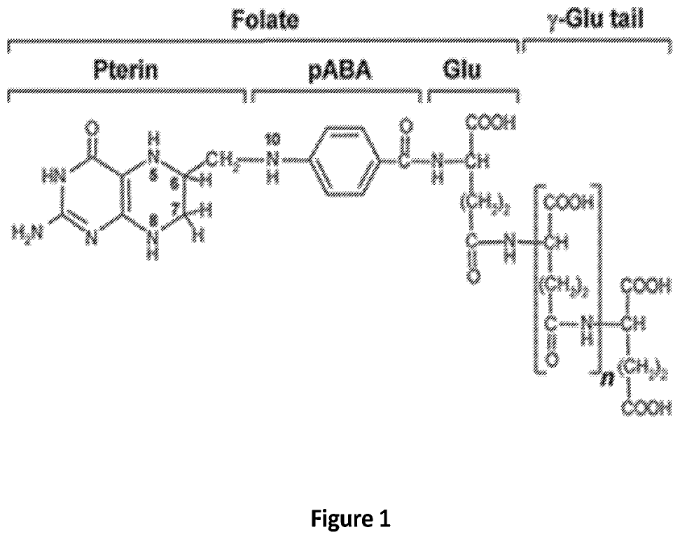

FIG. 2 shows schematic representation of an example of a folic acid operon consisting of L. lactis genes.

FIG. 3 shows schematic representation of an example of a folic acid operon consisting of A. gossypii genes.

FIG. 4 shows schematic representation of an example of a folic acid operon consisting of B. subtilis genes.

FIG. 5 shows schematic presentation of the FolC disruption cassettes with tetracycline resistance gene (TetR), heterologous folC2-LR or folC2-AG gene under P_{veg} promoter and flanking homology ends for native folC target gene disruption. The position of the primers used for PCR amplification of the DNA disruption cassette are denoted as lines.

FIG. 6 shows chromatogram of fermentation broth sample. Black: UV signal, red: MS scan signal.

FIG. 7 shows schematic representation of oxidation of 10-formyldihydrofolic acid to 10-formylfolic acid in the presence of oxygen, schematic representation of oxidation of 10-formyldihydrofolic acid to 10-formylfolic acid in the presence of hydrogen peroxide and schematic representation of oxidation of 10-formyldihydrofolic acid to 10-formylfolic acid in the presence of sodium periodate.

FIG. 8 shows schematic representation of deformylation of 10-formylfolic acid to folic acid in acidic medium.

FIG. 9 shows schematic representation of deformylation of 10-formylfolic acid to folic acid in alkaline medium.

FIG. 10 shows Folates production bioprocess profile. Folates (mg/L): full stars; Glucose concentration (g/L): empty squares; Acetoin concentration (g/L): full squares; PABA concentration (mg/L): empty circles; PABA feed (mg/L): vertical bars; Optical density: full circles.

FIG. 11 shows total folate production titers of B. subtilis strain w.t. 168, strain VBB38, strain FL21 and FL23 at the shaker 5 ml scale experiments.

FIG. 12 shows 5-methyl folate production titers of B. subtilis strain w.t. 168, strain VBB38, strain FL21, FL825 and FL2771 at the shaker 5 ml scale experiments.

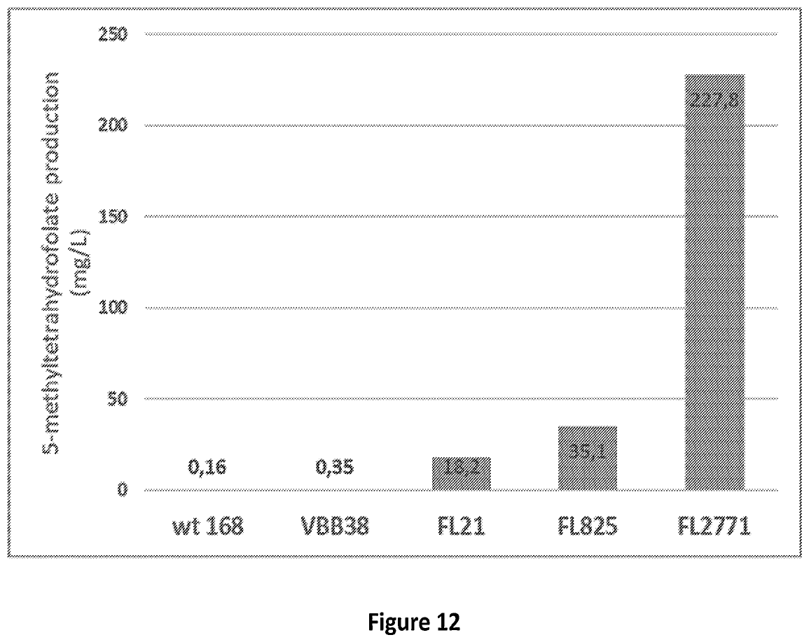

FIG. 13 shows a schematic presentation of 5-methyl-tetrahydrofolate producing strains development by transformation with artificial methyl-folate operon MTHF-OP-B into the parent strain FL2771.

FIG. 14 shoes a schematic presentation of the artificial MTHF-OP folate operons. Constructed operons have spectinomycin selectable marker and the homology for genome integration at ywhL locus. In the MTHF-OP-A operon genes (glyA, purU and yitJ) were selected from native organism B. subtilis and additional codon-optimized for optimal gene expression. Additionally, homologue gene (metF) from E. coli was used for construction of alternative to yitJ gene in operon MTHF-OP-B.

FIG. 15 shows transcription levels of metE gene in parent strain FL825 and the descendant strain FL2771.

FIG. 16 shows schematic representation of FOL-OP-BS1 a folic acid operon consisting of B. subtilis genes.

### DETAILED DESCRIPTION

Unless specifically defined herein, all technical and scientific terms used have the same meaning as commonly understood by a skilled artisan in the fields of biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); and Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986.

Genetically Engineered Microorganism of the Invention

In one aspect, the present invention thus provides a genetically engineered microorganism, such as genetically engineered bacterium. Suitably, the genetically engineered microorganism has the ability to produce 5-methylfolate (according to formula (I) shown below), and more specifically 5-methyl tetrahydrofolate (5-methyl-THF) (according to formula (II) shown below) including any stereoisomer thereof, such as enantiomer or diastereomer, such (6S)-5-Methyltetrahydrofolate (according to formula (IIa) shown below).

In some bacteria, such as Bacillus subtilis, the addition of L-glutamate to dihydropteroate (dihydrofolate synthetase (DHFS) activity, EC 6.3.2.12) and the subsequent additions of L-glutamate to tetrahydrofolate through gamma carboxyl groups (folylpolyglutamate synthetase (FPGS) activity, EC 6.3.2.17) are catalyzed by the same enzyme, FolC. In contrast, in eukaryotes and some other bacteria DHFS and FPGS enzymatic activities are encoded in different genes. B. subtilis, as many other bacteria, adds gamma-linked poly-glutamate tails to folates in order to increase solubility and prevent the loss of this essential cofactor into the environment. Thus, the Bacillus subtilis FolC possesses folyl-poly-glutamate synthetase (FPGS) activity which catalyzes the polyglutamylation of folates through their gamma-carboxyl groups in addition to its role as dihydrofolate synthase in the de novo folate biosynthetic pathway. The folate polyanions cannot be exported out of cells, resulting in enhanced intracellular retention (Sybesma et al., 2003c). In addition, the products of the FPGS enzyme, folyl-polyglutamates, are strong inhibitors of the folate biosynthetic enzymes (McGuire and Bertino, 1981). Therefore, in order to increase the production of folates, the present inventors have abolished the polyglutamylation of folates by knocking-out the native (endogenous) folC gene and replaced it with a heterologous folC gene encoding only for the essential dihydrofolate synthetase (DHFS) activity, resulting in the addition of only one essential glutamate moiety. Homologs of FolC with only the dihydrofolate synthetase (DHFS) and without folylpolyglutamate (FGPS) synthetase activity can be found in many bacteria species like Lactobacillus reuteri and many eukaryotic organisms like Ashbya gossypii.

After extensive and intensive research, the inventors have surprisingly found that if the expression level of an endogenous gene encoding a polypeptide having both dihydrofolate synthase activity and folylpolyglutamate synthetase activity, such as the gene folC, is reduced in a microorganism, and instead an exogenous gene is introduced encoding a polypeptide having only dihydrofolate synthase activity, only one glutamate is added on the biosynthesized folate, and the production capacity of a folate (such as 5-methyl-tetrahydrofolate), a salt, a precursor, or an intermediate thereof is thereby significantly increased.

Thus, the genetically engineered microorganism of the invention may been modified to have a decreased expression and/or activity of an endogenous polypeptide having both dihydrofolate synthase activity and folylpolyglutamate synthetase activity compared to an otherwise identical microorganism that does not carry said modification (reference microorganism).

According to some embodiments, the genetically engineered microorganism of the invention may been modified to have a decreased expression level of the endogenous gene encoding said polypeptide having both dihydrofolate synthase activity and folylpolyglutamate synthetase activity compared to an otherwise identical microorganism that does not carry said modification (reference microorganism). The expression level of the endogenous gene may, for example, be decreased by at least 50%, such as by at least 60%, at least 65%, at least 70%, at least 75%, at least 80%, at least 85%, at least 90%, at least 95% or at least 100% compared to the otherwise identical microorganism.

According to some embodiments, the endogenous gene encoding said polypeptide having both dihydrofolate synthase activity and folylpolyglutamate synthetase activity has been inactivated, such as by deletion of part of or the entire gene sequence.

According to some embodiments, the endogenous gene encoding said polypeptide having both dihydrofolate synthase activity and folylpolyglutamate synthetase activity has been inactivated by introducing or expressing in the microorganism a rare-cutting endonuclease able to selectively inactivating by DNA cleavage, preferably by double-strand break, the endogenous gene encoding said polypeptide. A rare-cutting endonuclease to be used in accordance of the present invention to inactivate the endogenous gene may, for instance, be a transcription activator-like effector (TALE) nuclease, meganuclease, zing-finger nuclease (ZFN), or RNA-guided endonuclease.

One way to inactivate the endogenous gene encoding said polypeptide having both dihydrofolate synthase activity and folylpolyglutamate synthetase activity is to use the CRISPRi system. The CRISPRi system was developed as a tool for targeted repression of gene expression or for blocking targeted locations on the genome. The CRISPRi system consists of the catalytically inactive, “dead” Cas9 protein (dCas9) and a guide RNA that defines the binding site for the dCas9 to DNA.

Thus, according to some embodiments, the endogenous gene encoding said polypeptide having both dihydrofolate synthase activity and folylpolyglutamate synthetase activity is inactivated by introducing or expressing in the microorganism a RNA-guided endonuclease, such as a catalytically inactive Cas9 protein, and a single guide RNA (sgRNA) specifically hybridizing (e.g. binding) under cellular conditions with the genomic DNA encoding a said polypeptide.

By way of example, if the endogenous gene encoding said polypeptide having both dihydrofolate synthase activity and folylpolyglutamate synthetase activity is to be inhibited in Bacillus subtilis, the single guide RNA (sgRNA) may comprise at least 20 consecutive nucleotides of SEQ ID NO: 5 or its complement.

According to some embodiments, the expression of an endogenous polypeptide having both dihydrofolate synthase activity and folylpolyglutamate synthetase activity is decreased by way of inhibition.

Inhibition of the expression of said endogenous polypeptide having both dihydrofolate synthase activity and folylpolyglutamate synthetase activity may be achieved by any suitable means known in the art. For example, the expression may be inhibited by gene silencing techniques involving the use of inhibitory nucleic acid molecules, such as antisense oligonucleotides, ribozymes or interfering RNA (RNAi) molecules, such as microRNA (miRNA), small interfering RNA (siRNA) or short hairpin RNA (shRNA).

According to some embodiments, the expression of said endogenous polypeptide having both dihydrofolate synthase activity and folylpolyglutamate synthetase activity is decreased (e.g., inhibited) by transcriptional and/or translational repression of the endogenous gene encoding said polypeptide.

According to some embodiments, the expression of said endogenous polypeptide having both dihydrofolate synthase activity and folylpolyglutamate synthetase activity is inhibited by introducing or expressing in the microorganism an inhibitory nucleic acid molecule. For example, the inhibitory nucleic acid molecule may be introduced by way of an exogenous nucleic acid molecule comprising a nucleotide sequence encoding said inhibitory nucleic acid molecule operably linked to a promoter, such as an inducible promoter, that is functional in the microorganism to cause the production of said inhibitory nucleic acid molecule. Suitably, the inhibitory nucleic acid molecule is one that specifically hybridizes (e.g. binds) under cellular conditions with cellular mRNA and/or genomic DNA encoding the endogenous polypeptide. Depending on the target, transcription of the encoding genomic DNA and/or translation of the encoding mRNA is/are inhibited.

According to some embodiments, the inhibitory nucleic acid molecule is an antisense oligonucleotide, ribozyme or interfering RNA (RNAi) molecule. Preferably, such nucleic acid molecule comprises at least 10 consecutive nucleotides of the complement of the cellular mRNA and/or genomic DNA encoding the polypeptide or enzyme of interest (e.g., the cellular mRNA and/or genomic DNA encoding the polypeptide.

By way of example, if the expression of an endogenous polypeptide having both dihydrofolate synthase activity and folylpolyglutamate synthetase activity is to be inhibited in Bacillus subtilis, such inhibitory nucleic acid molecule may comprise at least 10 consecutive nucleotides of the complement of SEQ ID NO: 5.

According to some embodiments, the inhibitory nucleic acid is an antisense oligonucleotide. Such antisense oligonucleotide is a nucleic acid molecule (either DNA or RNA) which specifically hybridizes (e.g. binds) under cellular conditions with the cellular mRNA and/or genomic DNA encoding the polypeptide.

According to some embodiments, the inhibitory nucleic acid molecule is a ribozyme, such as a hammerhead ribozyme. A ribozyme molecule is designed to catalytically cleave the mRNA transcript to prevent translation of the polypeptide.

According to some embodiments, the inhibitory nucleic acid molecule is an interfering RNA (RNAi) molecule. RNA interference is a biological process in which RNA molecules inhibit expression, typically causing the destruction of specific mRNA. Exemplary types of RNAi molecules include microRNA (miRNA), small interfering RNA (siRNA) and short hairpin RNA (shRNA). According to some embodiments, the RNAi molecule is a miRNA. According to some embodiments, the RNAi molecule is a siRNA. According to some embodiments, the RNAi molecule is a shRNA.

By way of example, if the expression of an endogenous polypeptide having both dihydrofolate synthase activity and folylpolyglutamate synthetase activity is to be inhibited in Bacillus subtilis, the RNAi molecule may be an interfering RNA complementary to SEQ ID NO: 5. The RNAi molecule may be a ribonucleic acid molecule comprising at least 10 consecutive nucleotides of the complement of SEQ ID NO: 5. The RNAi molecule may be a double-stranded ribonucleic acid molecule comprising a first strand identical to 20 to 25, such as 21 to 23, consecutive nucleotides of SEQ ID NO: 5, and a second strand complementary to said first strand.

According to some embodiments, the genetically engineered microorganism of the invention has been modified to have a decreased activity of an endogenous polypeptide having both dihydrofolate synthase activity and folylpolyglutamate synthetase activity compared to an otherwise identical microorganism that does not carry said modification (reference microorganism).

A decrease of the activity of the endogenous polypeptide having both dihydrofolate synthase activity and folylpolyglutamate synthetase activity may be achieved by any suitable means known in the art. For example, the activity may be decrease by introducing one or more mutations in the active site of the polypeptide resulting in the reduction or loss of activity. Thus, according to some embodiments, the activity of the endogenous polypeptide having both dihydrofolate synthase activity and folylpolyglutamate synthetase activity is decreased by at least one active-site mutation resulting in the reduction or loss of activity. The at least one active-site mutation may, for example, be at least one non-conservative amino acid substitution.

By way of example, if the activity of the endogenous polypeptide having both dihydrofolate synthase activity and folylpolyglutamate synthetase activity is to be decreased in Bacillus subtilis, the at least one active-site mutation may occur at any one of positions 51-54, 75, 114-117, 145, 152-154, 172, 263, 302 and 315 in the amino acid sequence set forth in SEQ ID NO: 11, which form part of the active site. In case of orthologous polypeptides, the at least one active-site mutation may be at a position which corresponds to any one of positions 51-54, 75, 114-117, 145, 152-154, 172, 263, 302 and 315 in the amino acid sequence set forth in SEQ ID NO: 11. According to some embodiments, the endogenous gene encoding said polypeptide having both dihydrofolate synthase activity and folylpolyglutamate synthetase activity is the gene folC.

According to some embodiments, the endogenous gene encoding said polypeptide having both dihydrofolate synthase activity and folylpolyglutamate synthetase activity comprises a nucleic acid sequence having at least 70%, such as at least 80%, at least 85%, at least 90%, at least 93%, at least 95%, at least 98% or at least 99%, sequence identity with the nucleic acid sequence set forth in SEQ ID NO: 5. According to come embodiments, the endogenous gene encoding a polypeptide having both dihydrofolate synthase activity and folylpolyglutamate synthetase activity comprises a nucleic acid sequence having at least 85%, such as at least 90%, sequence identity with the nucleic acid sequence set forth in SEQ ID NO: 5. According to come embodiments, the endogenous gene encoding a polypeptide having both dihydrofolate synthase activity and folylpolyglutamate synthetase activity comprises a nucleic acid sequence having at least 95%, such as at least 98%, sequence identity with the nucleic acid sequence set forth in SEQ ID NO: 5.

According to some embodiments, the polypeptide having both dihydrofolate synthase activity and folylpolyglutamate synthetase activity encoded by the endogenous gene comprises an amino acid which has at least 70%, such as at least 80%, at least 85%, at least 90%, at least 93%, at least 95%, at least 98% or at least 99%, sequence identity with the amino acid sequence set forth in SEQ ID NO: 11. According to some embodiments, the polypeptide having both dihydrofolate synthase activity and folylpolyglutamate synthetase activity encoded by the endogenous gene comprises an amino acid which has at least 85%, such as at least 90%, sequence identity with the amino acid sequence set forth in SEQ ID NO: 11. According to some embodiments, the polypeptide having both dihydrofolate synthase activity and folylpolyglutamate synthetase activity encoded by the endogenous gene comprises an amino acid which has at least 95%, such as at least 98%, sequence identity with the amino acid sequence set forth in SEQ ID NO: 11.

The genetically engineered microorganism of the invention may be (further) modified to express a heterologous polypeptide having only dihydrofolate synthase activity. The heterologous polypeptide having only dihydrofolate synthase activity may, for example, be derived from a bacterium or fungus, preferably selected from Lactobacillus reuteri and Ashbya gossypii.

According to some embodiments, the heterologous polypeptide having only dihydrofolate synthase activity comprises an amino acid sequence having at least 70%, such as at least 80%, at least 85%, at least 90%, at least 93%, at least 95%, at least 98% or at least 99%, sequence identity with SEQ ID NO: 22 or 23. According to some embodiments, the heterologous polypeptide having only dihydrofolate synthase activity comprises an amino acid sequence having at least 85%, such as at least 90%, sequence identity with SEQ ID NO: 22 or 23. According to some embodiments, the heterologous polypeptide having only dihydrofolate synthase activity comprises an amino acid sequence having at least 95%, such as at least 98%, sequence identity with SEQ ID NO: 22 or 23.

The folate molecule contains one pterin moiety, originating from guanosine triphosphate (GTP), bound to para-aminobenzoic acid (pABA) and at least one molecule of glutamic acid. Thus, de novo biosynthesis of folate requires three precursors: GTP, pABA and glutamic acid.

Folate biosynthesis proceeds via the conversion of GTP to the 6-hydroxymethyl-7,8-dihydropterin pyrophosphate (DHPPP) in four consecutive steps. The first step is catalyzed by GTP cyclohydrolase I (EC 3.5.4.16) (gene folE/mtrA) and involves an extensive transformation of GTP, to form a pterin ring structure. Following dephosphorylation, the pterin molecule undergoes aldolase (EC 4.1.2.25) (gene folB) and pyrophosphokinase reactions (EC 2.7.6.3) (gene folK), which produce the activated pyrophosphorylated DHPPP. Following the first condensation of para-aminobenzoic acid (pABA) with DHPPP catalyzed by dihydropteroate synthase (EC 2.5.1.15) (gene folP/sul) to produce dihydropteroate. The second condensation is reaction of glutamate with dihydropteroate to form dihydrofolate by dihydrofolate synthase (DHFS) (EC 6.3.2.12) (gene folC). Then, DHF is reduced by DHF reductase—DHFR (EC 1.5.1.3) (gene folA/dfrA) to the biologically active cofactor tetrahydrofolate (THF).

Proteins GlyA, PurU, YitJ and MetF are further involved in the tetrahydrofolate interconversion pathways. Tetrahydrofolate (THF) can be activated by serine hydroxymethyltransferase (gene glyA) (EC:2.1.2.1) by converting serine to glycine and subsequently transferring a methyl group to tetrahydrofolate, thus forming 5,10-methylene-tetrahydrofolate (5,10-mTHF). 5,10-mTHF is the major source of C1 units in the cell. Further in the tetrahydrofolate interconversion the B. subtilis yitJ gene and Escherichia coli metF gene are coding for 5,10-methylene-tetrahydrofolate reductase (EC 1.5.1.20), the enzyme that leads to the final formation of 5-methyltetrahydrofolate.

Additionally, enzyme PurU is also important for the tetrahydrofolate interconversion pathways, as it is involved as a formyltetrahydrofolate deformylase (EC 3.5.1.10) in the conversion of 10-formyltetrahydrofolate back to THF, to be further available for 5-methyltetrahydrofolate biosynthesis.

TABLE 1
Folate biosynthetic genes

| Gene | | Microorganism | Protein | Nucleotide |
|---|---|---|---|---|
| name/ | Enzymatic activity | (gene | sequence | sequence |
| synonym: | and EC number | source) | ID | ID |
| folC2-AG/ | dihydrofolate synthetase | Ashbya | SEQ ID | SEQ ID |
| FOL3 | EC: 6.3.2.12 | gossypii | NO: 23 | NO: 58 |
| folC2- | dihydrofolate synthetase | Lactobacillus | SEQ ID | SEQ ID |
| LR | EC: 6.3.2.12 | reuteri | NO: 22 | NO: 99 |
| folC | Bifunctional | Bacillus | SEQ ID | SEQ ID |
| | folylpolyglutamate synthase/ | subtilis | NO: 11 | NO: 5 |
| | dihydrofolate synthase | | | |
| | EC: 6.3.2.12/EC: 6.3.2.17 | | | |
| folE/ | GTP cyclohydrolase | Bacillus | SEQ ID | SEQ ID |
| mtrA | EC: 3.5.4.16 | subtilis | NO: 7 | NO: 1 |
| folB | 7,8-dihydroneopterin aldolase | Bacillus | SEQ ID | SEQ ID |
| | EC: 4.1.2.25 | subtilis | NO: 8 | NO: 2 |
| folK | 2-amino-4-hydroxy-6- | Bacillus | SEQ ID | SEQ ID |
| | hydroxymethyldihydropteridine | subtilis | NO: 9 | NO: 3 |
| | pyrophosphokinase | | | |
| | EC: 2.7.6.3 | | | |
| folP/ | dihydropteroate synthase | Bacillus | SEQ ID | SEQ ID |
| sul | EC: 2.5.1.15 | subtilis | NO: 10 | NO: 4 |
| folA/ | dihydrofolate reductase | Bacillus | SEQ ID | SEQ ID |
| dfrA | EC: 1.5.1.3 | subtilis | NO: 12 | NO: 6 |
| glyA | serine | Bacillus | SEQ ID | SEQ ID |
| | hydroxymethyltransferase | subtilis | NO: 79 | NO: 80 |
| | EC: 2.1.2.1 | | | |
| purU/ | formyltetrahydrofolate | Bacillus | SEQ ID | SEQ ID |
| ykkE | deformylase | subtilis | NO: 81 | NO: 82 |
| | EC: 3.5.1.10 | | | |
| yitJ | 5,10- | Bacillus | SEQ ID | SEQ ID |
| | methylenetetrahydrofolate | subtilis | NO: 83 | NO: 85 |
| | reductase | | | |
| | EC: 1.5.1.20 | | | |
| metF | 5,10- | Escherichia | SEQ ID | SEQ ID |
| | methylenetetrahydrofolate | coli | NO: 84 | NO: 86 |
| | reductase | | | |
| | EC: 1.5.1.20 | | | |
| metE | methionine synthase or 5- | Bacillus | SEQ ID | SEQ ID |
| | methyltetrahydropteroyltriglut | subtilis | NO: 100 | NO: 101 |
| | amate---homocysteine S- | | | |
| | methyltransferase | | | |

The present inventors have found that the introduction or up-regulation of one or more genes involved in the biosynthesis of 5-methyl-THF (such as, folE/mtrA, folB, folK, folP/sul, folA/dfrA, glyA, purU, yitJ and metF) in a microorganism can also significantly increase the production capacity of 5-methyl-THF, a salt, a precursor, or an intermediate thereof.

Thus, the genetically engineered microorganism of the invention may be (further) modified to have a significantly improved production capacity of a 5-methylfolate (such as 5-methyl-tetrahydrofolate) or a precursor or an intermediate thereof compared to an otherwise identical microorganism that does not carry said modification (reference microorganism). The production capacity of a 5-methylfolate (such as 5-methyl-tetrahydrofolate) or a precursor or an intermediate thereof may, for example, be increased by at least 50%, such as at least 100%, at least 200%, at least 500%, at least 1000%, at least 2000%, at least 5000%, at least 10000%, at least 20000% or at least 50000%, compared to the otherwise identical microorganism (reference microorganism).

According to some embodiments, the genetically engineered microorganism of the invention has been (further) modified to have an increased expression level of at least one gene (such as at least two, at least three, at least four, at least five, at least six, at least seven or at least eight genes) encoding an enzyme involved in the biosynthesis of a 5-methylfolate (such as 5-methyl-tetrahydrofolate) compared to an otherwise identical microorganism that does not carry said modification (reference microorganism). Thus, according to some embodiments, the genetically engineered microorganism of the invention has been (further) modified to have an increased expression level of at least one (such as at least two, at least three, at least four, at least five, at least six, at least seven or at least eight genes) enzymes involved in the biosynthesis of a 5-methylfolate (such as 5-methyl-tetrahydrofolate) compared to an otherwise identical microorganism that does not carry said modification (reference microorganism).

The expression level of the at least one gene (such as at least two, at least three, at least four, at least five, at least six, at least seven or at least eight genes) encoding an enzyme involved in the biosynthesis of a 5-methylfolate (such as 5-methyl-tetrahydrofolate) may, for example, be increased by at least 50%, at least 100 at least 200%, at least 500%, at least 1000%, at least 2000%, at least 5000%, at least 10000%, at least 20000% or at least 50000%, compared to the otherwise identical microorganism (reference microorganism).

According to some embodiments, the enzyme involved in the biosynthesis of a 5-methylfolate (such as 5-methyl-tetrahydrofolate) is selected from selected from the group consisting of: a polypeptide having GTP cyclohydrolase activity, a polypeptide having 7,8-dihydroneopterin aldolase activity, a polypeptide having 2-amino-4-hydroxy-6-hydroxymethyldihydropteridine pyrophosphokinase activity, a polypeptide having dihydropteroate synthase activity, a polypeptide having dihydrofolate reductase activity, a polypeptide having serine hydroxymethyltransferase activity, a polypeptide having formyltetrahydrofolate deformylase activity, and a polypeptide having 5,10-methylenetetrahydrofolate reductase activity.

According to some embodiments, the genetically engineered microorganism of the invention has been (further) modified to have an increased expression level of a gene encoding a polypeptide having GTP cyclohydrolase activity compared to an otherwise identical microorganism that does not carry said modification (reference microorganism). Thus, a genetically engineered microorganism of the invention may have an increased expression level of a polypeptide having GTP cyclohydrolase activity compared to an otherwise identical microorganism that does not carry said modification (reference microorganism).

According to some embodiments, the polypeptide having GTP cyclohydrolase activity comprises an amino acid sequence having at least 70%, such as at least 80%, at least 85%, at least 90%, at least 93%, at least 95%, at least 98% or at least 99%, sequence identity with SEQ ID NO: 7. According to some embodiments, the polypeptide having GTP cyclohydrolase activity comprises an amino acid sequence having at least 85%, such as at least 90%, sequence identity with SEQ ID NO: 7. According to some embodiments, the polypeptide having GTP cyclohydrolase activity comprises an amino acid sequence having at least 95%, such as at least 98%, sequence identity with SEQ ID NO: 7.

According to some embodiments, the genetically engineered microorganism of the invention has been (further) modified to have an increased expression level of a gene encoding a polypeptide having 7,8-dihydroneopterin aldolase activity compared to an otherwise identical microorganism that does not carry said modification (reference microorganism). Thus, a genetically engineered microorganism of the invention may have an increased expression level of a polypeptide having 7,8-dihydroneopterin aldolase activity compared to an otherwise identical microorganism that does not carry said modification (reference microorganism).

According to some embodiments, the polypeptide having 7,8-dihydroneopterin aldolase activity comprises an amino acid sequence having at least 70%, such as at least 80%, at least 85%, at least 90%, at least 93%, at least 95%, at least 98% or at least 99%, sequence identity with SEQ ID NO: 8. According to some embodiments, the polypeptide having 7,8-dihydroneopterin aldolase activity comprises an amino acid sequence having at least 85%, such as at least 90%, sequence identity with SEQ ID NO: 8. According to some embodiments, the polypeptide having 7,8-dihydroneopterin aldolase activity comprises an amino acid sequence having at least 95%, such as at least 98%, sequence identity with SEQ ID NO: 8.

According to some embodiments, the genetically engineered microorganism of the invention has been (further) modified to have an increased expression level of a gene encoding a polypeptide having 2-amino-4-hydroxy-6-hydroxymethyldihydropteridine pyrophosphokinase activity compared to an otherwise identical microorganism that does not carry said modification (reference microorganism). Thus, a genetically engineered microorganism of the invention may have an increased expression level of a polypeptide having 2-amino-4-hydroxy-6-hydroxymethyldihydropteridine pyrophosphokinase activity compared to an otherwise identical microorganism that does not carry said modification (reference microorganism).

According to some embodiments, the polypeptide having 2-amino-4-hydroxy-6-hydroxymethyl-dihydropteridine pyrophosphokinase activity comprises an amino acid sequence having at least 70%, such as at least 80%, at least 85%, at least 90%, at least 95%, at least 98% or at least 99%, sequence identity with SEQ ID NO: 9. According to some embodiments, the polypeptide having 2-amino-4-hydroxy-6-hydroxymethyl-dihydropteridine pyrophosphokinase activity comprises an amino acid sequence having at least 85%, such as at least 90%, sequence identity with SEQ ID NO: 9. According to some embodiments, the polypeptide having 2-amino-4-hydroxy-6-hydroxymethyl-dihydropteridine pyrophosphokinase activity comprises an amino acid sequence having at least 95%, such as at least 98%, sequence identity with SEQ ID NO: 9.

According to some embodiments, the genetically engineered microorganism of the invention has been (further) modified to have an increased expression level of a gene encoding a polypeptide having dihydropteroate synthase activity compared to an otherwise identical microorganism that does not carry said modification (reference microorganism). Thus, a genetically engineered microorganism of the invention may have an increased expression level of a polypeptide having dihydropteroate synthase activity compared to an otherwise identical microorganism that does not carry said modification (reference microorganism).

According to some embodiments, the polypeptide having dihydropteroate synthase activity comprises an amino acid sequence having at least 70%, such as at least 80%, at least 85%, at least 90%, at least 93%, at least 95%, at least 98% or at least 99%, sequence identity with SEQ ID NO: 10. According to some embodiments, the polypeptide having dihydropteroate synthase activity comprises an amino acid sequence having at least 85%, such as at least 90%, sequence identity with SEQ ID NO: 10. According to some embodiments, the polypeptide having dihydropteroate synthase activity comprises an amino acid sequence having at least 95%, such as at least 98%, sequence identity with SEQ ID NO: 10.

According to some embodiments, the genetically engineered microorganism of the invention has been (further) modified to have an increased expression level of a gene encoding a polypeptide having dihydrofolate reductase activity compared to an otherwise identical microorganism that does not carry said modification (reference microorganism). Thus, a genetically engineered microorganism of the invention may have an increased expression level of a polypeptide having dihydrofolate reductase activity compared to an otherwise identical microorganism that does not carry said modification (reference microorganism).

According to some embodiments, the polypeptide having dihydrofolate reductase activity comprises an amino acid sequence having at least 70%, such as at least 80%, at least 85%, at least 90%, at least 93%, at least 95%, at least 98% or at least 99%, sequence identity with SEQ ID NO: 12. According to some embodiments, the polypeptide having dihydrofolate reductase activity comprises an amino acid sequence having at least 85%, such as at least 90%, sequence identity with SEQ ID NO: 12. According to some embodiments, the polypeptide having dihydrofolate reductase activity comprises an amino acid sequence having at least 95%, such as at least 98%, sequence identity with SEQ ID NO: 12.

According to some embodiments, the genetically engineered microorganism of the invention has been (further) modified to have an increased expression level of a gene encoding a polypeptide having serine hydroxymethyltransferase activity compared to an otherwise identical microorganism that does not carry said modification (reference microorganism). Thus, a genetically engineered microorganism of the invention may have an increased expression level of a polypeptide having serine hydroxymethyltransferase activity compared to an otherwise identical microorganism that does not carry said modification (reference microorganism).

According to some embodiments, the polypeptide having serine hydroxymethyltransferase activity comprises an amino acid sequence having at least 70%, such as at least 80%, at least 85%, at least 90%, at least 93%, at least 95%, at least 98% or at least 99%, sequence identity with SEQ ID NO: 79. According to some embodiments, the polypeptide having serine hydroxymethyltransferase activity comprises an amino acid sequence having at least 85%, such as at least 90%, sequence identity with SEQ ID NO: 79. According to some embodiments, the polypeptide having serine hydroxymethyltransferase activity comprises an amino acid sequence having at least 95%, such as at least 98%, sequence identity with SEQ ID NO: 79.

According to some embodiments, the genetically engineered microorganism of the invention has been (further) modified to have an increased expression level of a gene encoding a polypeptide having formyltetrahydrofolate deformylase activity compared to an otherwise identical microorganism that does not carry said modification (reference microorganism). Thus, a genetically engineered microorganism of the invention may have an increased expression level of a polypeptide having formyltetrahydrofolate deformylase activity compared to an otherwise identical microorganism that does not carry said modification (reference microorganism).

According to some embodiments, the polypeptide having formyltetrahydrofolate deformylase activity comprises an amino acid sequence having at least 70%, such as at least 80%, at least 85%, at least 90%, at least 95%, at least 98% or at least 99%, sequence identity with SEQ ID NO: 81. According to some embodiments, the polypeptide having formyltetrahydrofolate deformylase activity comprises an amino acid sequence having at least 85%, such as at least 90%, sequence identity with SEQ ID NO: 81. According to some embodiments, the polypeptide having formyltetrahydrofolate deformylase activity comprises an amino acid sequence having at least 95%, such as at least 98%, sequence identity with SEQ ID NO: 81.

According to some embodiments, the genetically engineered microorganism of the invention has been (further) modified to have an increased expression level of a gene encoding a polypeptide having 5,10-methylenetetrahydrofolate reductase activity compared to an otherwise identical microorganism that does not carry said modification (reference microorganism). Thus, a genetically engineered microorganism of the invention may have an increased expression level of a polypeptide having 5,10-methylenetetrahydrofolate reductase activity compared to an otherwise identical microorganism that does not carry said modification (reference microorganism).

According to some embodiments, the polypeptide having 5,10-methylenetetrahydrofolate reductase activity comprises an amino acid sequence having at least 70%, such as at least 80%, at least 85%, at least 90%, at least 93%, at least 95%, at least 98% or at least 99%, sequence identity with SEQ ID NO: 83. According to some embodiments, the polypeptide having 5,10-methylenetetrahydrofolate reductase activity comprises an amino acid sequence having at least 85%, such as at least 90%, sequence identity with SEQ ID NO: 83. According to some embodiments, the polypeptide having 5,10-methylenetetrahydrofolate reductase activity comprises an amino acid sequence having at least 95%, such as at least 98%, sequence identity with SEQ ID NO: 83.

According to some embodiments, the polypeptide having 5,10-methylenetetrahydrofolate reductase activity comprises an amino acid sequence having at least 70%, such as at least 80%, at least 85%, at least 90%, at least 93%, at least 95%, at least 98% or at least 99%, sequence identity with SEQ ID NO: 84. According to some embodiments, the polypeptide having 5,10-methylenetetrahydrofolate reductase activity comprises an amino acid sequence having at least 85%, such as at least 90%, sequence identity with SEQ ID NO: 84. According to some embodiments, the polypeptide having 5,10-methylenetetrahydrofolate reductase activity comprises an amino acid sequence having at least 95%, such as at least 98%, sequence identity with SEQ ID NO: 84.

According to some embodiments, the at least one gene encoding an enzyme involved in the biosynthesis of a 5-methylfolate (such as 5-methyl-tetrahydrofolate) is selected from the group consisting of folE/mtrA, folB, folK, folP/sul, folA/dfrA, glyA, purU, yitJ and metF.

According to some embodiments, the at least one gene encoding an enzyme involved in the biosynthesis of a 5-methylfolate (such as 5-methyl-tetrahydrofolate) is heterologous to the genetically engineered microorganism.

According to some embodiments, the at least one gene encoding an enzyme involved in the biosynthesis of a 5-methylfolate is derived from a bacterium or fungus, preferably selected from the genus Bacillus, Escherichia, Lactococcus, Shewanella, Vibrio and Ashbya.

According to some embodiments, the at least one gene encoding an enzyme involved in the biosynthesis of a 5-methylfolate (such as 5-methyl-tetrahydrofolate) is derived from a bacterium or fungus selected from Bacillus subtilis, Lactobacillus lactis, Escherichia coli, Shewanella violacea, Vibrio natriegens or Ashbya gossypii.

The present inventors have further found that the down-regulation or deletion of an endogenous gene encoding a polypeptide having 5-methyltetrahydropteroyltriglutamate-homocysteine S-methyltransferase activity (such as the gene metE), which is the main enzyme that metabolizes/consumes the 5-methyltetrahydrofolate, in the microorganism can significantly further increase the accumulation and production capacity of a 5-methyltetrahydrofolate, a salt, a precursor, or an intermediate thereof.

Thus, the genetically engineered microorganism of the invention may be (further) modified to have a decreased expression level of an endogenous gene encoding a polypeptide having 5-methyltetrahydropteroyltriglutamate-homocysteine S-methyltransferase activity compared to an otherwise identical microorganism that does not carry said modification (reference microorganism). The expression level of the endogenous gene may, for example, be decreased by at least 50%, such as by at least 60%, at least 65%, at least 70%, at least 75%, at least 80%, at least 85%, at least 90%, at least 95% or at least 100% compared to the otherwise identical microorganism.

According to some embodiments, the genetically engineered microorganism comprises at least one mutation in the regulatory region of the endogenous gene encoding a polypeptide having 5-methyltetrahydropteroyltriglutamate-homocysteine S-methyltransferase activity, resulting in the decreased expression level. For example, the at least one mutation in the regulatory region may be at least one nucleotide substitution at a position in close proximity (e.g., 1 or 2 nucleotides up- or downstream) to the Pribnow box (TATAAT) sequence, resulting in the decreased expression level of the encoded polypeptide. As the particular case may be, for example, if the microorganism is Bacillus subtilis, the nucleotide substitution may be at a position located 12 nucleotides upstream from the start codon of the endogenous gene. The at least one nucleotide substitution may, for example, be a substitution of one type of purine by another type of purine (such as guanine to adenine).

According to some embodiments, the genetically engineered microorganism has been modified by replacing the endogenous promoter operatively linked to the endogenous gene encoding the polypeptide having 5-methyltetrahydropteroyltriglutamate-homocysteine S-methyltransferase activity with an exogenous promoter which is weaker in its affinity for RNA polymerase compared to the endogenous promoter. As will be appreciated by those skilled in the art, the weaker affinity for RNA polymerase will result in decreased levels of transcription, and hence decreased levels of the corresponding polypeptide being producing the microorganism.

According to some embodiments, the endogenous gene encoding a polypeptide having 5-methyltetrahydropteroyltriglutamate-homocysteine S-methyltransferase activity has been inactivated, such as by deletion of part of or the entire gene sequence.

According to some embodiments, the endogenous gene encoding said polypeptide having 5-methyltetrahydropteroyltriglutamate-homocysteine S-methyltransferase activity has been inactivated by introducing or expressing in the microorganism a rare-cutting endonuclease able to selectively inactivating by DNA cleavage, preferably by double-strand break, the endogenous gene encoding said polypeptide. A rare-cutting endonuclease to be used in accordance of the present invention to inactivate the endogenous gene may, for instance, be a transcription activator-like effector (TALE) nuclease, meganuclease, zing-finger nuclease (ZFN), or RNA-guided endonuclease.

One way to inactivate the endogenous gene encoding said polypeptide 5-methyltetrahydropteroyltriglutamate-homocysteine S-methyltransferase activity is to use the CRISPRi system as mentioned above. Thus, according to some embodiments, the endogenous gene encoding said polypeptide having 5-methyltetrahydropteroyltriglutamate-homocysteine S-methyltransferase activity is inactivated by introducing or expressing in the microorganism a RNA-guided endonuclease, such as a catalytically inactive Cas9 protein, and a single guide RNA (sgRNA) specifically hybridizing (e.g. binding) under cellular conditions with the genomic DNA encoding said polypeptide.

By way of example, if the endogenous gene encoding said polypeptide having 5-methyltetrahydropteroyltriglutamate-homocysteine S-methyltransferase activity is to be inhibited in Bacillus subtilis, the single guide RNA (sgRNA) may comprise at least 20 consecutive nucleotides of SEQ ID NO: 101 or its complement.

According to some embodiments, the expression of an endogenous polypeptide having 5-methyltetrahydropteroyltriglutamate-homocysteine S-methyltransferase activity is decreased by way of inhibition.

Inhibition of the expression of said endogenous polypeptide having 5-methyltetrahydropteroyltriglutamate-homocysteine S-methyltransferase activity may be achieved by any suitable means known in the art. For example, the expression may be inhibited by gene silencing techniques involving the use of inhibitory nucleic acid molecules, such as antisense oligonucleotides, ribozymes or interfering RNA (RNAi) molecules, such as microRNA (miRNA), small interfering RNA (siRNA) or short hairpin RNA (shRNA).

According to some embodiments, the expression of said endogenous polypeptide having 5-methyltetrahydropteroyltriglutamate-homocysteine S-methyltransferase activity is decreased (e.g., inhibited) by transcriptional and/or translational repression of the endogenous gene encoding said polypeptide.

According to some embodiments, the expression of said endogenous polypeptide having 5-methyltetrahydropteroyltriglutamate-homocysteine S-methyltransferase activity is inhibited by introducing or expressing in the microorganism an inhibitory nucleic acid molecule. For example, the inhibitory nucleic acid molecule may be introduced by way of an exogenous nucleic acid molecule comprising a nucleotide sequence encoding said inhibitory nucleic acid molecule operably linked to a promoter, such as an inducible promoter, that is functional in the microorganism to cause the production of said inhibitory nucleic acid molecule. Suitably, the inhibitory nucleic acid molecule is one that specifically hybridizes (e.g. binds) under cellular conditions with cellular mRNA and/or genomic DNA encoding the endogenous polypeptide.

According to some embodiments, the inhibitory nucleic acid molecule is an antisense oligonucleotide, ribozyme or interfering RNA (RNAi) molecule. Preferably, such nucleic acid molecule comprises at least 10 consecutive nucleotides of the complement of the cellular mRNA and/or genomic DNA encoding the polypeptide.

By way of example, if the expression of an endogenous polypeptide having 5-methyltetrahydropteroyltriglutamate-homocysteine S-methyltransferase activity is to be inhibited in Bacillus subtilis, such inhibitory nucleic acid molecule may comprise at least 10 consecutive nucleotides of the complement of SEQ ID NO: 101.

According to some embodiments, the inhibitory nucleic acid is an antisense oligonucleotide. Such antisense oligonucleotide is a nucleic acid molecule (either DNA or RNA) which specifically hybridizes (e.g. binds) under cellular conditions with the cellular mRNA and/or genomic DNA encoding the polypeptide.

According to some embodiments, the inhibitory nucleic acid molecule is a ribozyme, such as a hammerhead ribozyme. A ribozyme molecule is designed to catalytically cleave the mRNA transcript to prevent translation of the polypeptide.

According to some embodiments, the inhibitory nucleic acid molecule is an interfering RNA (RNAi) molecule. According to some embodiments, the RNAi molecule is a miRNA. According to some embodiments, the RNAi molecule is a siRNA. According to some embodiments, the RNAi molecule is a shRNA.

By way of example, if the expression of an endogenous polypeptide having 5-methyltetrahydropteroyltriglutamate-homocysteine S-methyltransferase activity is to be inhibited in Bacillus subtilis, the RNAi molecule may be an interfering RNA complementary to SEQ ID NO: 101. The RNAi molecule may be a ribonucleic acid molecule comprising at least 10 consecutive nucleotides of the complement of SEQ ID NO: 101. The RNAi molecule may be a double-stranded ribonucleic acid molecule comprising a first strand identical to 20 to 25, such as 21 to 23, consecutive nucleotides of SEQ ID NO: 101, and a second strand complementary to said first strand.

According to some embodiments, the genetically engineered microorganism of the invention has been modified to have a decreased activity of an endogenous polypeptide having 5-methyltetrahydropteroyltriglutamate-homocysteine S-methyltransferase activity compared to an otherwise identical microorganism that does not carry said modification (reference microorganism).

A decrease of the activity of the endogenous polypeptide having 5-methyltetrahydropteroyltriglutamate-homocysteine S-methyltransferase activity may be achieved by any suitable means known in the art. For example, the activity may be decreased by introducing one or more mutations in the active site of the polypeptide resulting in the reduction or loss of activity. Thus, according to some embodiments, the activity of the endogenous polypeptide having 5-methyltetrahydropteroyltriglutamate-homocysteine S-methyltransferase activity is decreased by at least one active-site mutation resulting in the reduction or loss of activity. The at least one active-site mutation may, for example, be at least one non-conservative amino acid substitution.

By way of example, if the activity of the endogenous polypeptide having 5-methyltetrahydropteroyltriglutamate-homocysteine S-methyltransferase activity is to be decreased in Bacillus subtilis, the at least one active-site mutation may occur at any one of positions 18, 21, 112, 117, 119, 435-437, 488, 494, 519-521, 565, 601, 603, 605, 645, 647, 669, 730 and 731 in the amino acid sequence set forth in SEQ ID NO: 100, which form part of the active site. In case of orthologous polypeptides, the at least one active-site mutation may be at a position which corresponds to any one of positions 18, 21, 112, 117, 119, 435-437, 488, 494, 519-521, 565, 601, 603, 605, 645, 647, 669, 730 and 731 in the amino acid sequence set forth in SEQ ID NO: 100. According to some embodiments, the endogenous gene encoding a polypeptide having 5-methyltetrahydropteroyltriglutamate-homocysteine S-methyltransferase activity is the gene metE.

According to some embodiments, the endogenous gene encoding a polypeptide having 5-methyltetrahydropteroyltriglutamate-homocysteine S-methyltransferase activity comprises a nucleic acid sequence having at least 70%, such as at least 80%, at least 85%, at least 90%, at least 93%, at least 95%, at least 98% or at least 99%, sequence identity with the nucleic acid sequence set forth in SEQ ID NO: 101. According to some embodiments, the endogenous gene encoding a polypeptide having 5-methyltetrahydropteroyltriglutamate-homocysteine S-methyltransferase activity comprises a nucleic acid sequence having at least 85%, such as at least 90%, sequence identity with the nucleic acid sequence set forth in SEQ ID NO: 101. According to some embodiments, the endogenous gene encoding a polypeptide having 5-methyltetrahydropteroyltriglutamate-homocysteine S-methyltransferase activity comprises a nucleic acid sequence having at least 95%, such as at least 98%, sequence identity with the nucleic acid sequence set forth in SEQ ID NO: 101.

According to some embodiments, the polypeptide having polypeptide having 5-methyltetrahydropteroyltriglutamate-homocysteine S-methyltransferase activity encoded by the endogenous gene comprises an amino acid which has at least 70%, such as at least 80, at least 85%, at least 90%, at least 93%, at least 95%, at least 98% or at least 99%, sequence identity with the amino acid sequence set forth in SEQ ID NO: 100. According to some embodiments, the polypeptide having polypeptide having 5-methyltetrahydropteroyltriglutamate-homocysteine S-methyltransferase activity encoded by the endogenous gene comprises an amino acid which has at least 85%, such as at least 90%, sequence identity with the amino acid sequence set forth in SEQ ID NO: 100.

According to some embodiments, the polypeptide having polypeptide having 5-methyltetrahydropteroyltriglutamate-homocysteine S-methyltransferase activity encoded by the endogenous gene comprises an amino acid which has at least 95%, such as at least 98%, sequence identity with the amino acid sequence set forth in SEQ ID NO: 100.

Generally, a microorganism as referred to herein may be any suitable microorganism, including single-celled or multicellular microorganisms such as bacteria or yeast.

Bacterial microorganisms may be Gram-positive or Gram-negative bacteria. Non-limiting examples for Gram-negative bacteria include species from the genera Escherichia, Erwinia, Klebsiella and Citrobacter. Non-limiting examples of Gram-positive bacteria include species from the genera Bacillus, Lactococcus, Lactobacillus, Geobacillus, Pediococcus, Moorella, Clostridium, Corynebacterium, Streptomyces, Streptococcus, and Cellulomonas.

According to some embodiments, the microorganism is a bacterium, which may be a bacterium of the genus Bacillus, Lactococcus, Lactobacillus, Clostridium, Corynebacterium, Geobacillus, Streptococcus, Pediococcus, Moorella, Pseudomonas, Streptomyces, Escherichia, Shigella, Acinetobacter, Citrobacter, Salmonella, Klebsiella, Enterobacter, Erwinia, Kluyvera, Serratia, Cedecea, Morganella, Hafnia, Edwardsiella, Providencia, Proteus, or Yersinia.

According to some embodiments, the microorganism is a bacterium of the genus Escherichia. A non-limiting example of a bacterium of the genus Escherichia is Escherichia coli. According to some embodiments, the microorganism is Escherichia coli.

According to some embodiments, the microorganism is a bacterium of the genus Bacillus. Non-limiting examples of a bacterium of the genus Bacillus are Bacillus subtilis, Bacillus amyloliquefaciens, Bacillus licheniformis, and Bacillus mojavensis. According to some embodiments, the microorganism is Bacillus subtilis.

Yeast cells may be derived from e.g., Saccharomyces, Pichia, Schizosacharomyces, Zygosaccharomyces, Hansenula, Pachyosolen, Kluyveromyces, Debaryomyces, Yarrowia, Candida, Cryptococcus, Komagataella, Lipomyces, Rhodospiridium, Rhodotorula, or Trichosporon.

According to some embodiments, the microorganism is a yeast of the genus Saccharomyces. A non-limiting example of a yeast of the genus Saccharomyces is Saccharomyces cerevisiae. According to certain embodiments, the microorganism is Saccharomyces cerevisiae.

As noted above, a genetically engineered microorganism of the invention may be modified to express one or more polypeptides as detailed herein, which means that one or more exogenous nucleic acid molecules, such as DNA molecules, which comprise(s) a nucleotide sequence or nucleotide sequences encoding said polypeptide or polypeptides has been introduced in the microorganism. Techniques for introducing exogenous nucleic acid molecule, such as a DNA molecule, into the various host cells are well-known to those of skill in the art, and include transformation (e.g., heat shock or natural transformation), transfection, conjugation, electroporation, microinjection and microparticle bombardment.

Accordingly, a genetically engineered microorganism of the invention may comprise an exogenous nucleic acid molecule comprising a nucleotide sequence encoding a polypeptide as detailed herein. In order to facilitate expression of a polypeptide in the microorganism, the exogenous nucleic acid molecule may comprise suitable regulatory elements such as a promoter that is functional in the host cell to cause the production of an mRNA molecule and that is operably linked to the nucleotide sequence encoding said polypeptide. Promoters useful in accordance with the invention are any known promoters that are functional in a given host cell to cause the production of an mRNA molecule. Many such promoters are known to the skilled person. Such promoters include promoters normally associated with other genes, and/or promoters isolated from any bacteria, yeast, fungi, alga or plant cell. The use of promoters for protein expression is generally known to those of skilled in the art of molecular biology, for example, see Sambrook et al., Molecular cloning: A Laboratory Manual, Cold Spring Harbor Laboratory, Cold Spring Harbor, N.Y., 1989. Besides a promoter, the exogenous nucleic acid molecule may further comprise at least one regulatory element selected from a 5′ untranslated region (5′UTR) and 3′ untranslated region (3′ UTR). Many such 5′ UTRs and 3′ UTRs derived from prokaryotes and eukaryotes are well known to the skilled person.

The exogenous nucleic acid molecule may be a vector or part of a vector, such as an expression vector. Normally, such a vector remains extrachromosomal within the microorganism which means that it is found outside of the nucleus or nucleoid region of the microorganism. It is also contemplated by the present invention that the exogenous nucleic acid molecule is stably integrated into the genome of the host cell. Means for stable integration into the genome of a microorganism, e.g., by homologous recombination, are well known to the skilled person.

It is understood that the details given herein with respect to a genetically engineered microorganism apply to other aspects of the invention, in particular to the methods according to the invention, which are described in more detail below.

### Methods of the Invention

In a second aspect, the present invention provides a method for preparing a folate, precursor or intermediate thereof. Particularly, a method for preparing a folate, precursor or intermediate thereof comprises:
i) cultivating a genetically engineered microorganism according to the invention in a culture medium under suitable culture conditions to obtain a fermentation product containing said folate, precursor or intermediate thereof; and ii) optionally, separating and/or purifying said folate, precursor or intermediate thereof.

The medium employed may be any conventional medium suitable for culturing the host cell in question, and may be composed according to the principles of the prior art. The medium will usually contain all nutrients necessary for the growth and survival of the respective host cell, such as carbon and nitrogen sources and other inorganic salts. Suitable media, e.g. minimal or complex media, are available from commercial suppliers, or may be prepared according to published receipts, e.g. the American Type Culture Collection (ATCC) Catalogue of strains. Non-limiting standard medium well known to the skilled person include Luria Bertani (LB) broth, Sabouraud Dextrose (SD) broth, MS broth, Yeast Peptone Dextrose, BMMY, GMMY, or Yeast Malt Extract (YM) broth, which are all commercially available. A non-limiting example of suitable media for culturing bacterial cells, such as B. subtilis or E. coli cells, including minimal media and rich media such as Luria Broth (LB), M9 media, M17 media, SA media, MOPS media, Terrific Broth, YT and others. Suitable media for culturing eukaryotic cells, such as yeast cells, are RPMI 1640, MEM, DMEM, all of which may be supplemented with serum and/or growth factors as required by the particular host cell being cultured. The medium for culturing eukaryotic cells may also be any kind of minimal media such as Yeast minimal media.

Suitable conditions for culturing the respective microorganism are well known to the skilled person. Typically, the genetically engineered microorganism is cultured at a temperature ranging from 32 to about 42° C., preferably in a range from 34 to 39° C., more preferably in a range from 36 to 39° C., such as at about 37° C. The pH of the medium may be in a range from 6 to 8, preferably in a range from 6.5 to 7.5, more preferably in a range from 6.8 to 7.2. The cultivation in step i) may be carried out for a period in the range from 10 to 70 h, preferably in a range from 24 to 60 h, more preferably in a range from 36 to 50 h.

The method may further comprise ii) separating and/or purifying said folate, precursor or intermediate thereof. The folate, precursor or intermediate thereof may be separated and/or purified by any conventional method for isolation and purification chemical compounds from a medium. Well-known purification procedures include centrifugation or filtration, precipitation, and chromatographic methods such as e.g. ion exchange chromatography, gel filtration chromatography, etc.

The folate prepared by the method of the invention is preferably a compound of Formula I:

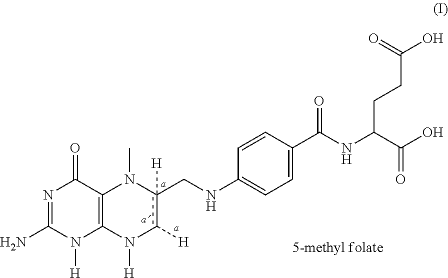

optionally in form of one of the stereoisomers, preferably enantiomers or diastereomers, in form of a racemate or in form of a mixture of at least two of the stereoisomers, preferably enantiomers and/or diastereomers, in any mixing ratio.

In the generic formula (I) above, it is meant that when a is single bond, a′ is none, or when a′ is a single bond, a is none.

According to certain embodiments, the folate prepared by the method of the invention is compound of Formula II (5-methyltetrahydrofolate):

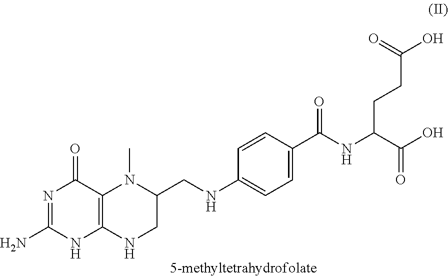 [SMILES: CN1c2c([nH]c(N)nc2=O)NCC1CNc1ccc(C(=O)NC(CCC(=O)O)C(=O)O)cc1]

optionally in form of one of the stereoisomers, preferably enantiomers or diastereomers, in form of a racemate or in form of a mixture of at least two of the stereoisomers, preferably enantiomers and/or diastereomers, in any mixing ratio.

According to particular embodiments, the folate prepared by the method of the invention is compound of Formula IIa (L-5-methyltetrahydrofolate; (6S)-5-methyltetrahydrofolate):

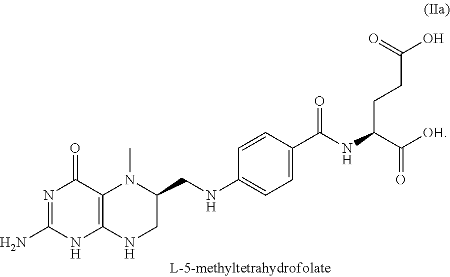 [SMILES: CN1c2c([nH]c(N)nc2=O)NC[C@@H]1CNc1ccc(C(=O)N[C@@H](CCC(=O)O)C(=O)O)cc1]

According to certain embodiments, the folate prepared by the method of the invention is compound of Formula III (5-methyldihydrofolate):

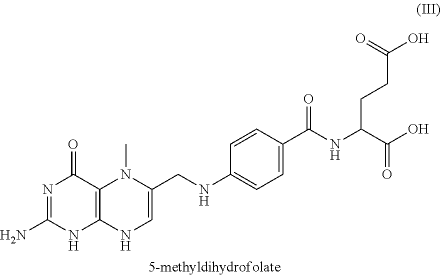 [SMILES: CN1C(CNc2ccc(C(=O)NC(CCC(=O)O)C(=O)O)cc2)=CNc2[nH]c(N)nc(=O)c21]

optionally in form of one of the stereoisomers, preferably enantiomers or diastereomers, in form of a racemate or in form of a mixture of at least two of the stereoisomers, preferably enantiomers and/or diastereomers, in any mixing ratio.

The inventors have also surprisingly found that the addition of para-aminobenzoic acid (PABA) during the cultivation of the strain, obtained as described above, can significantly further increase the production capacity of a folate, a salt, a precursor, or an intermediate thereof. Thus, according to some embodiments, the method further comprises the step of adding para-aminobenzoic acid (PABA) during the cultivation step (i).

The para-aminobenzoic acid (PABA) may, for example, be a PABA selected from the group consisting of: potassium para-aminobenzoate, sodium para-aminobenzoate, methyl para-aminobenzoate, ethyl para-aminobenzoate, butyl para-aminobenzoate, or a combination thereof.

According to some embodiments, the method further comprises subjecting the product obtained in the steps (i) or (ii) to acidic or alkaline conditions to further obtain a derivative compound.

In a further aspect, the present invention provides a method of preparing a genetically engineered microorganism of the present invention. Particularly, the method of preparing a genetically engineered microorganism of the present invention comprises any one (such as all) of the steps (a) to (d) below:
(a) increased the expression level of at least one (such as at least two, at least three, at least four, at least five, at least six, at least seven or at least eight) enzyme involved in the biosynthesis of a 5-methylfolate (such as 5-methyl-tetrahydrofolate) compared to an otherwise identical microorganism (reference microorganism);
(b) decreasing the expression and/or activity of an endogenous polypeptide having 5-methyltetrahydropteroyltriglutamate-homocysteine S-methyltransferase activity compared to an otherwise identical microorganism that does not carry said modification (reference microorganism);
(c) decreasing the expression and/or activity of an endogenous polypeptide having both dihydrofolate synthase activity and folylpolyglutamate synthetase activity compared to an otherwise identical microorganism that does not carry said modification (reference microorganism); and/or
(d) expressing a heterologous polypeptide having only dihydrofolate synthase activity.

According to some embodiments, the method comprises any one of the steps (a) to (b) below:
(a) increased the expression level of at least one enzyme involved in the biosynthesis of a 5-methylfolate compared to an otherwise identical microorganism (reference microorganism); and
(b) decreasing the expression level of an endogenous gene encoding a polypeptide having 5-methyltetrahydropteroyltriglutamate-homocysteine S-methyltransferase activity compared to an otherwise identical microorganism (reference microorganism);

optionally further comprising the steps (c) and (d) below:
(c) decreasing the expression level of the endogenous gene encoding a polypeptide having both dihydrofolate synthase activity and folylpolyglutamate synthetase activity compared to an otherwise identical microorganism (reference microorganism); and
(d) expressing a heterologous polypeptide having only dihydrofolate synthase activity.

According to some embodiments, the method of preparing a genetically engineered microorganism of the present invention comprises the steps of aa) introducing into said microorganism at least one exogenous nucleic acid molecule comprising a nucleic acid sequence encoding an enzyme involved in the biosynthesis of a 5-methylfolate (such as 5-methyl-tetrahydrofolate); bb) inactivating, such as by deleting part of or the entire gene sequence, the endogenous gene encoding a polypeptide having 5-methyltetrahydropteroyltriglutamate-homocysteine S-methyltransferase activity or introducing at least one mutation in the regulatory region of said endogenous gene, which results in the decrease expression level; cc) inactivating, such as by deleting part of or the entire gene sequence, the endogenous gene encoding a polypeptide having both dihydrofolate synthase activity and folylpolyglutamate synthetase activity in said microorganism; and/or dd) introducing into said microorganism an exogenous nucleic acid molecule comprising a nucleic acid sequence encoding a heterologous polypeptide having only dihydrofolate synthase activity.

### Certain Definitions

5-methylfolate as used herein means any one of 5-methyltetrahydrofolate and 5-methyldihydrofolate including any stereoisomer form and protonated (acid) or deprotonated (salt) form thereof.

The phrase “ability to produce 5-methlyfolate” means that the microorganism, such as a bacterium, is able to produce, excrete or secrete, and/or cause accumulation of 5-methylfolate in a culture medium or in the microorganism when the microorganism is cultured in the medium. A microorganism may be considered as having the ability to produce 5-methlyfolate, if it expresses all enzymes involved in the biosynthetic pathway resulting in 5-methlyfolate.

The phrase “ability to produce 5-methyl-tetrahydrofolate (5-methyl-THF)” means that the microorganism, such as a bacterium, is able to produce, excrete or secrete, and/or cause accumulation of 5-methyl-THF in a culture medium or in the microorganism when the microorganism is cultured in the medium. A microorganism may be considered as having the ability to produce 5-methyl-THF, if it expresses all enzymes involved in the biosynthetic pathway resulting in 5-methyl-THF.

As used herein, a “polypeptide having both dihydrofolate synthase activity and folylpolyglutamate synthetase activity” means a polypeptide that catalyzes the reaction: ATP+7,8-dihydropteroate+L-glutamate<=>ADP+phosphate+7,8-dihydropteroylglutamate (EC 6.3.2.12) and the reaction: ATP+tetrahydropteroyl-(gamma-Glu)(n)+L-glutamate<=>ADP+phosphate+tetrahydropteroyl-(gamma-Glu)(n+1) (EC 6.3.2.17). A polypeptide having both dihydrofolate synthase activity and folylpolyglutamate synthetase activity is, for example, encoded by the gene folC found in, e.g., Bacillus subtilis. Further information regarding folC of, e.g., Bacillus subtilis is available at KEGG (https://www.kegg.jp/kegg/genes.html) under Accession number BSU28080. See also NCBI Reference Sequence: NP_390686.1 for the amino acid sequence (B. subtilis).

As used herein, a “polypeptide having only dihydrofolate synthase activity” means a polypeptide that catalyzes only the reaction: ATP+7,8-dihydropteroate+L-glutamate<=>ADP+phosphate+7,8-dihydropteroylglutamate (EC 6.3.2.12). A polypeptide having only dihydrofolate synthase activity is, for example, encoded by the gene folC2 found in, e.g., Ashbya gossypii and Lactobacillus reuteri. Further information regarding folC2 of, e.g., Ashbya gossypii and Lactobacillus reuteri is available at KEGG (https://www.kegg.jp/kegg/genes.html) under Accession number AGOS_AEL310C and Lreu_1277, respectively. See also NCBI Reference Sequence: NP_984550.1 (Ashbya gossypii) and WP 003668526.1 (Lactobacillus reuteri) for the amino acid sequence.

As used herein, a “polypeptide having GTP cyclohydrolase activity” means a polypeptide that catalyzes the reaction: GTP+H(2)O<=>formate+2-amino-4-hydroxy-6-(erythro-1,2,3-trihydroxypropyl)-dihydropteridine triphosphate (EC 3.5.4.16). A polypeptide having GTP cyclohydrolase activity is, for example, encoded by the gene folE found in, e.g., Bacillus subtilis. Further information regarding folE of, e.g., Bacillus subtilis is available at KEGG (https://www.kegg.jp/kegg/genes.html) under Accession number BSU22780. See also NCBI Reference Sequence: NP_390159.1 for the amino acid sequence (B. subtilis).

As used herein, a “polypeptide having 7,8-dihydroneopterin aldolase activity” means a polypeptide that catalyzes the reaction: 7,8-dihydroneopterin<=>6-hydroxymethyl-7,8-dihydropterin+glycolaldehyde (EC 4.1.2.25). A polypeptide having 7,8-dihydroneopterin aldolase activity is, for example, encoded by the gene folB found in, e.g., Bacillus subtilis. Further information regarding folB of, e.g., Bacillus subtilis is available at KEGG (https://www.kegg.jp/kegg/genes.html) under Accession number BSU00780. See also NCBI Reference Sequence: NP_387959.1 for the amino acid sequence (B. subtilis).

As used herein, a “polypeptide having 2-amino-4-hydroxy-6-hydroxymethyldihydropteridine pyrophosphokinase activity” means a polypeptide that catalyzes the reaction: ATP+6-hydroxymethyl-7,8-dihydropterin<=>AMP+6-hydroxymethyl-7,8-dihydropterin diphosphate (EC 2.7.6.3). A polypeptide having 2-amino-4-hydroxy-6-hydroxymethyldihydropteridine pyrophosphokinase activity is, for example, encoded by the gene folK found in, e.g., Bacillus subtilis. Further information regarding folK of, e.g., Bacillus subtilis is available at KEGG (https://www.kegg.jp/kegg/genes.html) under Accession number BSU00790. See also NCBI Reference Sequence: NP_387960.1 for the amino acid sequence (B. subtilis).

As used herein, a “polypeptide having dihydropteroate synthase activity” means a polypeptide that catalyzes the reaction: 6-hydroxymethyl-7,8-dihydropterin diphosphate+4-aminobenzoate<=>diphosphate+dihydropteroate (EC 2.5.1.15). A polypeptide having dihydropteroate synthase activity is, for example, encoded by the gene folP found in, e.g., Bacillus subtilis. Further information regarding folP of, e.g., Bacillus subtilis is available at KEGG (https://www.kegg.jp/kegg/genes.html) under Accession number BSU00770. See also NCBI Reference Sequence: NP_387958.1 for the amino acid sequence (B. subtilis).

As used herein, a “polypeptide having dihydrofolate reductase activity” means a polypeptide that catalyzes the reaction: 5,6,7,8-tetrahydrofolate+NADP(+)<=>7,8-dihydrofolate+NADPH (EC 1.5.1.3). A polypeptide having dihydrofolate reductase activity is, for example, encoded by the gene folA found in, e.g., Bacillus subtilis. Further information regarding folA of, e.g., Bacillus subtilis is available at KEGG (https://www.kegg.jp/kegg/genes.html) under Accession number BSU21810. See also NCBI Reference Sequence: NP_390064.1 for the amino acid sequence (B. subtilis).

As used herein, a “polypeptide having serine hydroxymethyltransferase activity” means a polypeptide that catalyzes the reaction: 5,10-methylenetetrahydrofolate+glycine+H(2)O<=>tetrahydrofolate+L-serine (EC 2.1.2.1). A polypeptide having serine hydroxymethyltransferase activity is, for example, encoded by the gene glyA found in, e.g., Bacillus subtilis. Further information regarding glyA of, e.g., Bacillus subtilis is available at KEGG (https://www.kegg.jp/kegg/genes.html) under Accession number BSU36900. See also NCBI Reference Sequence: NP_391571.1 for the amino acid sequence (B. subtilis).

As used herein, a “polypeptide having formyltetrahydrofolate deformylase activity” means a polypeptide that catalyzes the reaction: 10-formyltetrahydrofolate+H(2)O<=>formate+tetrahydrofolate (EC 5.5.1.10). A polypeptide having formyltetrahydrofolate deformylase activity is, for example, encoded by the gene purU found in, e.g., Bacillus subtilis. Further information regarding purU of, e.g., Bacillus subtilis is available at KEGG (https://www.kegg.jp/kegg/genes.html) under Accession number BSU13110. See also NCBI Reference Sequence: NP_389194.2 for the amino acid sequence (B. subtilis).

As used herein, a “polypeptide having 5,10-methylenetetrahydrofolate reductase activity” means a polypeptide that catalyzes the reaction: 5-methyltetrahydrofolate+NAD(P)(+)<=>5,10-methylenetetrahydrofolate+NAD(P)H (EC 1.5.1.20). A polypeptide having 5,10-methylenetetrahydrofolate reductase activity is, for example, encoded by the gene yitJ found in, e.g., Bacillus subtilis. Further information regarding yitJ of, e.g., Bacillus subtilis is available at KEGG (https://www.kegg.jp/kegg/genes.html) under Accession number BSU11010. See also NCBI Reference Sequence: NP_388982.1 for the amino acid sequence (B. subtilis). A polypeptide having 5,10-methylenetetrahydrofolate reductase activity is, for example, encoded by the gene metF found in, e.g., Escherichia coli. Further information regarding metF of, e.g., Escherichia coli is available at KEGG (https://www.kegg.jp/kegg/genes.html) under Accession number b3941. See also NCBI Reference Sequence: NP_418376.1 for the amino acid sequence (B. subtilis).

As used herein, a “polypeptide having 5-methyltetrahydropteroyltriglutamate-homocysteine S-methyltransferase activity” means a polypeptide that catalyzes the reaction: 5-methyltetrahydropteroyltri-L-glutamate+L-homocysteine<=>tetrahydropteroyltri-L-glutamate+L-methionine (EC 2.1.1.14). A polypeptide having 5-methyltetrahydropteroyltriglutamate-homocysteine S-methyltransferase activity is, for example, encoded by the gene metE found in, e.g., Bacillus subtilis. Further information regarding metE of, e.g., Bacillus subtilis is available at KEGG (https://www.kegg.jp/kegg/genes.html) under Accession number BSU13180. See also NCBI Reference Sequence: NP_389201.2 for the amino acid sequence (B. subtilis).

“Heterologous” or “exogenous” as used herein in the context of a gene or nucleic acid molecule refer to a gene or nucleic acid molecule (i.e. DNA or RNA molecule) that does not occur naturally as part of the genome of the microorganism in which it is present or which is found in a location or locations in the genome that differ from that in which it occurs in nature. Thus, a “heterologous” or “exogenous” gene or nucleic acid molecule is not endogenous to the microorganism and has been exogenously introduced into the microorganism. A “heterologous” gene or nucleic acid molecule DNA molecule may be from a different organism, a different species, a different genus or a different kingdom, as the host DNA.

“Heterologous” as used herein in the context of a polypeptide (such as an enzyme) means that a polypeptide is normally not found in or made (i.e. expressed) by the host microorganism, but derived from a different organism, a different species, a different genus or a different kingdom.

As used herein, the term “ortholog” or “orthologs” refers to genes, nucleic acid molecules encoded thereby, i.e., mRNA, or proteins encoded thereby that are derived from a common ancestor gene but are present in different species.

By “decreased expression level” of a gene it is meant that the amount of the transcription product, respectively the amount of the polypeptide encoded by said gene produced by the genetically engineered microorganism is decreased compared to an otherwise identical microorganism that does not carry said modification. More particularly, by “decreased expression level” of a gene it is meant that the amount of the transcription product, respectively the amount of the polypeptide encoded by said gene produced by the genetically engineered microorganism is decreased by at least 10%, such as at least 20%, at least 30%, at least 40%, at least 50% at least 60%, at least 70%, at least 80%, at least 90% or at least 100%, compared to an otherwise identical microorganism that does not carry said modification. The level of expression of a gene can be determined by well-known methods, including PCR, Southern blotting, and the like. In addition, the level of gene expression can be estimated by measuring the amount of mRNA transcribed from the gene using various well-known methods, including Northern blotting, quantitative RT-PCR, and the like. The amount of the polypeptide encoded by the gene can be measured by well-known methods, including ELISA, Immunohistochemistry or Western Blotting and the like.

Expression of a gene can be decreased by introducing a mutation into the gene in the genome of the microorganism so that the intracellular activity of the polypeptide encoded by the gene is decreased as compared to an otherwise identical microorganism that does not carry said mutation. Mutations which result in a decreased expression of the gene include the replacement of one nucleotide or more to cause an amino acid substitution in the polypeptide encoded by the gene (missense mutation), introduction of a stop codon (nonsense mutation), deletion or insertion of nucleotides to cause a frame shift, insertion of a drug-resistance gene, or deletion of a part of the gene or the entire gene (Qiu and Goodman, 1997; Kwon et al., 2000). Expression can also be decreased by modifying an expression regulating sequence such as the promoter, the Shine-Dalgarno (SD) sequence, etc. Expression of the gene can also be decreased by gene replacement (Datsenko and Wanner, 2000), such as the “lambda-red mediated gene replacement”. The lambda-red mediated gene replacement is a particularly suitable method to inactive one or more genes as described herein.

“Inactivating”, “inactivation” and “inactivated”, when used in the context of a gene, means that the gene in question no longer expresses a functional protein. It is possible that the modified DNA region is unable to naturally express the gene due to the deletion of a part of or the entire gene sequence, the shifting of the reading frame of the gene, the introduction of missense/nonsense mutation(s), or the modification of an adjacent region of the gene, including sequences controlling gene expression, such as a promoter, enhancer, attenuator, ribosome-binding site, etc. Preferably, a gene of interest is inactivated by deletion of a part of or the entire gene sequence, such as by gene replacement. Inactivation may also be accomplished by introducing or expressing a rare-cutting endonuclease able to selectively inactivating by DNA cleavage, preferably by double-strand break, the gene of interest. A “rare-cutting endonuclease” within the context of the present invention includes transcription activator-like effector (TALE) nucleases, meganucleases, zing-finger nucleases (ZFN), and RNA-guided endonucleases.

The presence or absence of a gene in the genome of a microorganism, such as a bacterium, can be detected by well-known methods, including PCR, Southern blotting, and the like. In addition, the level of gene expression can be estimated by measuring the amount of mRNA transcribed from the gene using various well-known methods, including Northern blotting, quantitative RT-PCR, and the like. The amount of the protein encoded by the gene can be measured by well-known methods, including SDS-PAGE followed by an immunoblotting assay (Western blotting analysis), and the like.

By “increased expression level” of a gene it is meant that the amount of the transcription product, respectively the amount of the polypeptide encoded by said gene produced by the genetically engineered microorganism is increased compared to an otherwise identical microorganism that does not carry said modification. More particularly, by “increased expression level” of a gene it is meant that the amount of the transcription product, respectively the amount of the polypeptide encoded by said gene produced by the genetically engineered microorganism is increased by at least 10%, such as at least 20%, at least 30%, at least 40%, at least 50% at least 60%, at least 70%, at least 80%, at least 90%, at least 100%, at least 150%, at least 200%, at least 300%, at least 400%, at least 500%, at least 600%, at least 700% at least 800%, at least about 900%, at least about 1000%, at least about 2000%, at least about 3000%, at least about 4000%, at least about 5000%, at least about 6000%, at least about 7000%, at least about 8000% at least about 9000% or at least about 10000%, compared to an otherwise identical microorganism that does not carry said modification. The level of expression of a gene can be determined by well-known methods, including PCR, Southern blotting, and the like. In addition, the level of gene expression can be estimated by measuring the amount of mRNA transcribed from the gene using various well-known methods, including Northern blotting, quantitative RT-PCR, and the like. The amount of the polypeptide encoded by the gene can be measured by well-known methods, including ELISA, Immunohistochemistry or Western Blotting and the like.

By “increased expression level” of a polypeptide it is meant that the amount of the polypeptide in question produced by the genetically engineered microorganism is increased compared an otherwise identical microorganism that does not carry said modification. More particularly, by “increased expression level” of a polypeptide it is meant that the amount of the polypeptide in question produced by the genetically engineered microorganism is increased by at least 10%, such as at least 20%, at least 30%, at least 40%, at least 50% at least 60%, at least 70%, at least 80%, at least 90%, at least 100%, at least 150%, at least 200%, at least 300%, at least 400%, at least 500%, at least 600%, at least 700% at least 800%, at least about 900%, at least about 1000%, at least about 2000%, at least about 3000%, at least about 4000%, at least about 5000%, at least about 6000%, at least about 7000%, at least about 8000% at least about 9000% or at least about 10000%, compared an otherwise identical microorganism that does not carry said modification. The amount of a polypeptide produced in a given cell can be determined by any suitable quantification technique known in the art, such as ELISA, Immunohistochemistry or Western Blotting.

An increase in polypeptide expression may be achieved by any suitable means well-known to those skilled in the art. For example, an increase in polypeptide expression may be achieved by increasing the number of copies of the gene or genes encoding the polypeptide in the microorganism, such as by introducing into the microorganism an exogenous nucleic acid, such as a vector, comprising the gene or genes encoding the polypeptide operably linked to a promoter that is functional in the microorganism to cause the production of an mRNA molecule. An increase in polypeptide expression may also be achieved by integration of at least a second copy of the gene or genes encoding the polypeptide into the genome of the microorganism. An increase in polypeptide expression may also be achieved by increasing the strength of the promoter(s) operably linked to the gene or genes encoding the polypeptide. An increase in polypeptide expression may also be achieved by modifying the ribosome binding site on the mRNA molecule encoding the polypeptide. By modifying the sequence of the ribosome binding site the translation initiation rate may be increased, thus increasing the translation efficiency.

As used herein, “decreased”, “decreasing” or “decrease of” expression of a polypeptide (such as a polypeptide having both dihydrofolate synthase activity and folylpolyglutamate synthetase activity or a polypeptide having 5-methyltetrahydropteroyltriglutamate-homocysteine S-methyltransferase activity) means that the expression of said polypeptide in a modified microorganism is reduced compared to the expression of said polypeptide in an otherwise identical microorganism that does not carry said modification (control). The expression of a polypeptide in a modified microorganism may be reduced by at least about 10%, and preferably by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% or 100%, or any percentage, in whole integers between 10% and 100% (e.g., 6%, 7%, 8%, etc.), compared to the expression of said polypeptide in an otherwise identical microorganism that does not carry said modification (control). More particularly, “decreased”, “decreasing” or “decrease of” expression of a polypeptide means that the amount of the polypeptide in the microorganism is reduced by at least about 10%, and preferably by at least about 20%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% or 100%, or any percentage, in whole integers between 10% and 100% (e.g., 6%, 7%, 8%, etc.), compared to the amount of said polypeptide in an otherwise identical microorganism that does not carry said modification (control). The expression or amount of a polypeptide in a microorganism can be determined by any suitable means know in the art, including techniques such as ELISA, Immunohistochemistry, Western Blotting or Flow Cytometry.

As used herein, “decreased”, “decreasing” or “decrease of” activity of a polypeptide (such as a polypeptide having both dihydrofolate synthase activity and folylpolyglutamate synthetase activity or a polypeptide having 5-methyltetrahydropteroyltriglutamate-homocysteine S-methyltransferase activity) means that the catalytic activity of said polypeptide in a modified microorganism is reduced compared to the catalytic activity of said polypeptide in an otherwise identical microorganism that does not carry said modification (control). The activity of a polypeptide in a modified microorganism may be reduced by at least about 10%, and preferably by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% or 100%, or any percentage, in whole integers between 10% and 100% (e.g., 6%, 7%, 8%, etc.), compared to the expression of said polypeptide in an otherwise identical microorganism that does not carry said modification (control). The activity of a polypeptide in a microorganism can be determined by any suitable protein and enzyme activity assay.

As used herein, “regulatory region” of a gene refers to a nucleic acid sequence that affect the expression of a coding sequence. Regulatory regions are known in the art and include, but are not limited to, promoters, enhancers, transcription terminators, polyadenylation sites, matrix attachment regions and/or other elements that regulate expression of a coding sequence.

As used herein, “expression” includes any step involved in the production of a polypeptide (e.g., encoded enzyme) including, but not limited to, transcription, post-transcriptional modification, translation, post-translational modification, and secretion.

“Substitution” or “substituted” refers to modification of the polypeptide by replacing one amino acid residue with another, for instance the replacement of an Serine residue with a Glycine or Alanine residue in a polypeptide sequence is an amino acid substitution. When used with reference to a polynucleotide, “substitution” or “substituted” refers to modification of the polynucleotide by replacing one nucleotide with another. For instance the replacement of a cytosine with a thymine in a polynucleotide sequence is a nucleotide substitution.

“Non-conservative substitution”, when used with reference to a polypeptide, refers to a substitution of an amino acid in a polypeptide with an amino acid with significantly differing side chain properties. Non-conservative substitutions may use amino acids between, rather than within, the defined groups and affects (a) the structure of the peptide backbone in the area of the substitution (e.g., serine for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Percentage of sequence identity,” “% sequence identity” and “percent identity” are used herein to refer to comparisons between an amino acid sequence and a reference amino acid sequence. The “% sequence identify”, as used herein, is calculated from the two amino acid sequences as follows: The sequences are aligned using Version 9 of the Genetic Computing Group's GAP (global alignment program), using the default BLOSUM62 matrix with a gap open penalty of −12 (for the first null of a gap) and a gap extension penalty of −4 (for each additional null in the gap). After alignment, percentage identity is calculated by expressing the number of matches as a percentage of the number of amino acids in the reference amino acid sequence.

Where a numerical limit or range is stated herein, the endpoints are included. Also, all values and sub ranges within a numerical limit or range are specifically included as if explicitly written out.

Having generally described this invention, a further understanding can be obtained by reference to certain specific examples, which are provided herein for purposes of illustration only, and are not intended to be limiting unless otherwise specified.

### EXAMPLES

### Example 1: Identification of Folate Biosynthetic Genes in the Genome of Bacillus subtilis

Genes and enzymes involved in the folate biosynthetic pathway are known in the literature and are described in detail in the KEGG database (www.genome.jp/kegg/pathway.html). Nucleotide and protein sequences of key folate biosynthetic genes of B. subtilis were obtained by investigating the genome and protein databases of B. subtilis using the BLAST algorithm. Sequences of folate biosynthetic genes and enzymes were introduced as “query” and the corresponding B. subtilis sequences were identified as “hits.” Sequences of folate biosynthetic genes are presented in Table 2 below.

TABLE 2
Genes and enzymes involved in folate biosynthesis in Bacillus subtilis.

| | | | | Nucleotide sequence |
|---|---|---|---|---|
| | | NCBI | Protein | *optimized sequence |
| Gene | | accession | sequence | for Bacillus subtilis |
| name: | Enzymatic activity | number | ID | codon usage |
| folE | GTP cyclohydrolase | NP_390159 | SEQ ID | SEQ ID |
| | (Bacillus subtilis) | | NO: 7 | NO: 13 |
| folB | 7,8-dihydroneopterin | NP_387959 | SEQ ID | SEQ ID |
| | aldolase | | NO: 8 | NO: 14 |
| | (Bacillus subtilis) | | | |
| folK | 2-amino-4-hydroxy-6- | NP_387960 | SEQ ID | SEQ ID |
| | hydroxymethyldihydropteridine | | NO: 9 | NO: 15 |
| | pyrophosphokinase | | | |
| | (Bacillus subtilis) | | | |
| sul | dihydropteroate | NP_387958 | SEQ ID | SEQ ID |
| | synthase | | NO: 10 | NO: 16 |
| | (Bacillus subtilis) | | | |
| folC | bifunctional | NP_390686 | SEQ ID | / |
| | folylpolyglutamate | | NO: 11 | |
| | synthetase/ | | | |
| | dihydrofolate | | | |
| | synthetase (Bacillus | | | |
| | subtilis) | | | |
| dfrA | dihydrofolate reductase | NP_390064 | SEQ ID | SEQ ID |
| | (Bacillus subtilis) | | NO: 12 | NO: 17 |
| glyA | serine | NP_391571 | SEQ ID | SEQ ID |
| | hydroxymethyltransferase | | NO: 79 | NO: 91 |
| | (Bacillus subtilis) | | | |
| purU | formyltetrahydrofolate | NP_389194 | SEQ ID | SEQ ID |
| | deformylase (Bacillus | | NO: 81 | NO: 92 |
| | subtilis) | | | |
| yitJ | 5,10- | NP_388982 | SEQ ID | SEQ ID |
| | methylenetetrahydrofolate | | NO: 83 | NO: 93 |
| | reductase | | | |
| | (Bacillus subtilis) | | | |

### Example 2: Synthesis of Synthetic Genes for Folic Acid Biosynthesis, Optimized for Bacillus subtilis

The amino acid sequences (SEQ ID NOs: 7, 8, 9, 10, 12, 79, 81 and 83) were used for gene codon optimization (Codon Optimization Tool from IDT Integrated DNA Technologies) in order to improve protein expression in B. subtilis. The synthesized DNA fragments (SEQ ID NOs: 13, 14, 15, 16, 17, 91, 92 and 93, respectively) were designed with addition of RBS sequences, regulatory promoter sequence (such as p15 SEQ ID NO:38) for gene overexpression and short adapter sequences at both ends needed for further assembly of folic acid operon expression cassette.

### Example 3: Assembly of Folic Acid Operons

Folic Acid Operon Assembled from Bacillus subtilis Genes

Key folate biosynthetic genes from Bacillus subtilis genes synthesized as DNA fragments (SEQ ID NOs: 13, 14, 15, 16 and 17) were used for assembly of folic acid operon (FOL-OP-BS2). For integration of folic acid operon into B. subtilis genome two additional DNA fragments with lacA homologies and erythromycin selectable marker (SEQ ID NOs: 18 and 19) were designed and synthesized for stabile genome integration.

In the first step of the folic acid operon assembly PCR amplification of separate DNA fragments was performed with specific set of primers (primer pair SEQ ID NO: 26 and SEQ ID NO: 27 for fragment SEQ ID NO: 13; primer pair SEQ ID NO: 32 and SEQ ID NO: 28 for fragment SEQ ID NO: 17; primer pair SEQ ID NO: 33 and SEQ ID NO: 29 for fragment SEQ ID NO:15; primer pair SEQ ID NO: 34 and SEQ ID NO: 30 for fragment SEQ ID NO: 16; primer pair SEQ ID NO:35 and SEQ ID NO: 31 for fragment SEQ ID NO: 14).

Fragments were amplified using Eppendorf cycler and Phusion polymerase (Thermo Fisher) with buffer provided by the manufacturer with addition of 200 μM dNTPs, 5% DMSO, 0.5 μM of each primer and approximately 20 ng of template in a final volume of 50 μl for 32 cycles.

Used program: 98° C. 2 min
32 cycles of (98° C. 30 s, 65° C. 15 s, 72° C. 30 s)
72° C. 5 min
10° C. hold

PCR of each fragment was run on 0.8% agarose gel and cleaned from gel by protocol provided in Wizard PCR cleaning kit (Promega). The fragments were assembled into artificial folate operon by repetitive steps of restriction and ligation. A combination of NdeI and AseI restriction sites were used in order to assure compatible restriction ends for successful ligation. After each step of ligation, the combined fragments were used as a new template for next PCR amplification. Restriction was done in 50 μl volume with addition of 5 μl FD green buffer, 3 μl of selected enzyme and approximately 1500 ng of PCR fragment. Fragments were cleaned after restriction with Wizard SV Gel and PCR Clean-up system and first two were used in ligation. We used 2.5 U T4 DNA ligase (Thermo Fisher) with buffer provided by manufacturer and addition of 5% PEG 4000 and both fragments in 1:1 molar ratio to final volume 15 μl. In the next step 1 μl of inactivated ligation was used as a template in new 50 μL PCR with primers SEQ ID NO: 26 and SEQ ID NO:28 and same program (with longer elongation time) and mix as used above. PCR was run on 0.8% agarose gel, fragment was excised from gel and cleaned. Cleaned new fragment (assembly of SEQ ID NO: 13 and SEQ ID NO: 17) was cut with AseI restriction enzyme and after additional cleaning used in ligation with third fragment (SEQ ID NO: 15), already cut with NdeI and cleaned after. Following new PCR on ligation as a template, we also added fragment four and five by same protocol to make fragment of up to five folate biosynthetic genes.

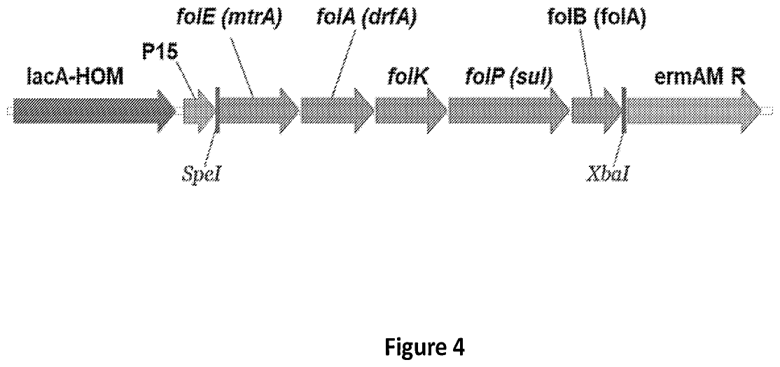

Constructed folic acid operon assembled from Bacillus subtilis genes (shown in FIG. 4), was used for transformation (see Example 5) in order to generate strain FL722, after cultivation measurements of total folate was performed (see Example 12).

Folic Acid Operon from Lactococcus lactis Subsp. lactis Genes

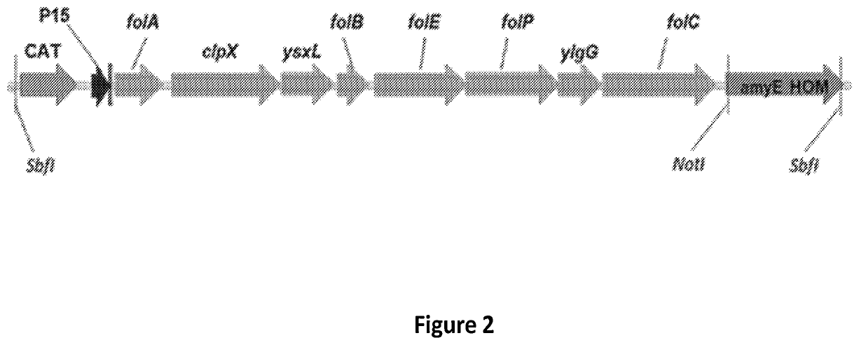

Heterologous genes (folA, clpX, ysxL, folB, folE, folP, ylgG and folC) from Lactococcus lactis subsp. lactis operon FOL-OP-LL (SEQ ID NO: 49) were amplified by PCR and isolated genomic DNA was used as a template. Primers for PCR amplification were designed for two separate PCR reactions, where in the 1^{st} PCR reaction primers (SEQ ID NO:45 and SEQ ID NO:46) were used for specific amplification of genes from genomic DNA and in the 2^{nd} PCR reaction primers (SEQ ID NO:47 and SEQ ID NO:48) were used to additionally restriction sites (NheI and NotI) were introduced at both ends of the operon. The PCR product was subcloned into a low copy vector pFOL1 and the strong constitutive promoter P₁₅ (SEQ ID NO: 38) was added at the start of the FOL-OP-LL operon. For construction of integration cassette for FOL-OP-LL operon, chloramphenicol resistance cassette and downstream homology for amyE locus was introduced. In the final step, the integration cassette was realised from cloning vector by using SbfI restriction enzyme and used for self-ligation to achieve multi copy genome integration. Constructed folic acid operon assembled from Lactococcus lactis subsp. lactis genes (shown in FIG. 2), was used for transformation in order to generate strain FL84, after cultivation measurements of total folate was performed (see Example 12).

Folic Acid Operon FOL-OP-BS1 Assembled from Bacillus subtilis Genes

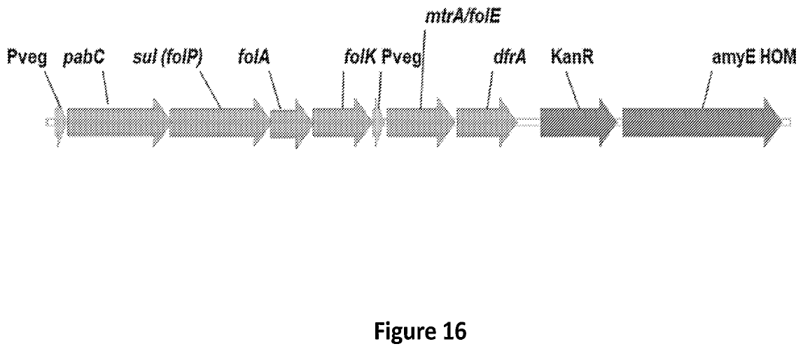

Assembly of FOL-OP-BS1 (artificial folate operon) was carried out from two separate DNA fragments of the synthesis technology (BS-FOLOP1-COMB and BS-FOLOP2-COMB, SEQ ID NOs: 77 and 78). Synthetic DNA fragment BS-FOLOP2-COMB was cloned into low-copy-number plasmids bearing kanamycin resistance cassette and downstream homology for amyE locus. In the final step of constructing the FOL-OP-BS1 integration cassette, the assembly was done in vitro using Gibson assembly protocol with specifically designed primer pair (SEQ ID NOs: 87 and 88) for amplification of BS-FOLOP1-COMB (part A) and primer pair (SEQ ID NO 89 and 90) for amplification of BS-FOLOP2-COMB+KnR+amyE-HOM (part B). Operon FOL-OP-BS1 for folate biosynthesis is under the expression of a strong constitutive promoter Pveg and kanamycin-resistance cassette as a selective marker (FIG. 16). Assembled integration cassette FOL-OP-BS1 was PCR amplified and further used for self-ligation to enable multi-copy genome integration into the B. subtilis genome at amyE locus.

Folic Acid Operon from Ashbya gossypii (Eremothecium gossypii) Genes

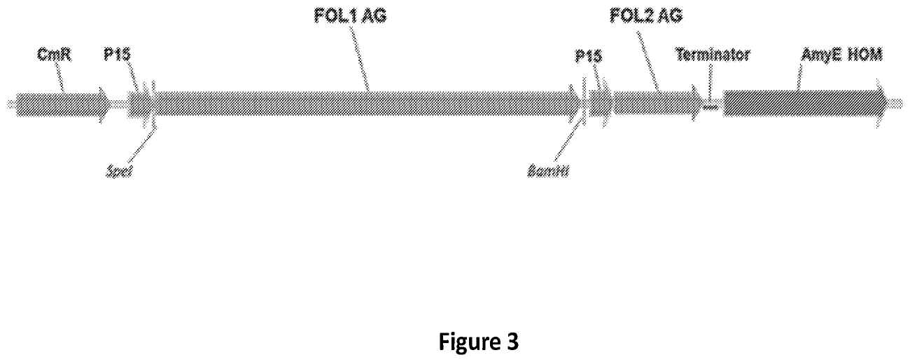

The expression cassette (FOL-OP-AG) from Ashbya gossypii (Eremothecium gossypii), a known B2 vitamin-producing filamentous fungus, was constructed using two synthetic folate biosynthesis genes, fol1-AG (SEQ ID NO:50) and fol2-AG (SEQ ID NO:51). The genes were codon-optimized for B. subtilis optimal expression and synthesized as two separate DNA fragments FOL1-AG (SEQ ID NO:52) and FOL2-AG (SEQ ID NO:53) where additional regulatory promoter sequence (promoter P₁₅) was introduced. The FOL1-AG fragment was first subcloned into a low copy vector pFOL1 using SpeI/BamHI restriction sites downstream of the chloramphenicol resistance cassette and strong constitutive promoter P₁₅. In the second step the FOL2-AG fragment was subcloned into a low copy vector pFOL2 upstream of the homology for amyE locus using EcoRV restriction site. In the next step DNA fragment containing P₁₅-fol2-AG and amyE homology was PCR amplified using primers (SEQ ID NO:54 and SEQ ID NO:55) and cloned into plasmid pFOL1 downstream of the chloramphenicol resistance cassette and P₁₅-fol1-AG using BamHI restriction site. In the final step, the assembled integration cassette FOL-OP-AG was PCR amplified using primers (SEQ ID NO:56 and SEQ ID NO:57) and PCR product was used for transformation of the cell. Constructed folic acid operon assembled from Ashbya gossypii genes (shown in FIG. 3), was used for transformation in order to generate strain FL260, after cultivation measurements of total folate was performed (see Example 12).

### Example 4: Assembly of Genetic Construct for folC Replacement

In order to replace the native folylpolyglutamate synthase (folC), which is capable of attaching multiple glutamate residues to folates, with the variant, capable of attaching only the first glutamate residue in folate biosynthesis we set out to generate the corresponding genetic constructs. The folC disruption cassettes were assembled by using folC homology ends amplified by PCR from gDNA B. subtilis VBB38 by using the corresponding primer pairs SEQ ID NO:43 and SEQ ID NO:44. PCR mix was made with Phusion polymerase (Thermo Fisher) and buffer provided by manufacturer with addition of 5% DMSO, 200 μM dNTPs and 0.5 μM of each primer to final volume of 50 μL for 32 cycles (annealing temperature 65° C., elongation time 2 min). The amplified PCR fragment was excised from 0.8% agarose gel, cleaned with Wizard Gel and PCR Clean-up system kit and phosphorylated with T4 polynucleotide kinase (Thermo Fisher) in buffer A, provided by manufacturer, with addition of 1 mM ATP.

Prepared fragment was ligated in low copy plasmid pET-29c (Novagen), which was previously cut with FspAl and Xhol, blunt-ended with DNA polymerase 1, Large (Klenow) fragment (Thermo Fisher) and dephosphorylated with FastAP Thermosensitive Alkaline Phosphatase (Thermo Fisher).

Tetracycline resistance cassette (SEQ ID NO:21) was used to disrupt folC gene sequence. Tetracycline resistance cassette was inserted into folC sequence by cutting plasmid with Bsp119l restriction enzyme, blunt-ended with DNA polymerase 1, Large (Klenow) fragment (Thermo Fisher), dephosphorylated, using FastAP and ligated using T4 DNA ligase (Thermo Fisher).

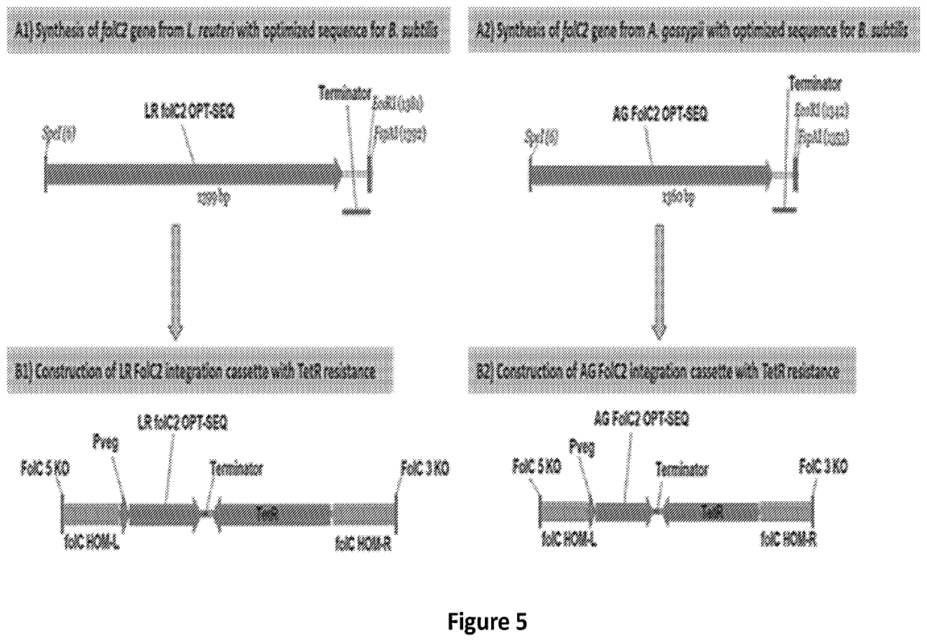

Further, heterologous folC2 protein sequences from Lactobacillus reuteri (folC2-LR) (SEQ ID NO:22) and from Ashbya gossypii (folC2-AG) (SEQ ID NO:23) were used for design codon optimized DNA sequence for folC2-LR (Lactobacillus reuteri) (SEQ ID NO:24) and for folC2-AG (Ashbya gossypii) (SEQ ID NO:25) heterologous gene expression. DNA fragments were synthesized (IDT Integrated DNA Technologies) and used for construction of two integration cassettes (shown in FIG. 5). First, we generated a blunt-ended fragment containing the Pveg promotor (SEQ ID NO:37) using DNA polymerase 1, Large (Klenow) fragment (Thermo Fisher) and ligated it in the plasmid with folC homology, previously cut with Xbal and blunt-ended with DNA polymerase 1, Large (Klenow) fragment (Thermo Fisher).

Next, newly constructed plasmid was cut with Bcul and FspAl restriction enzymes and dephosphorylated, using FastAP. After that, plasmid was ligated with ordered optimized sequences folC2-AG in folC2-LR, previously cut with Bcul and FspAl restriction enzymes. In this plasmid tetracycline resistance, previously cut with EcoRl restriction enzyme and blunt-ended, was ligated, after restriction of plasmid with FspAl and dephosphorylated. Constructed plasmids were used as a template for PCR primers SEQ ID NO:43 and SEQ ID NO:44 in order to generate folC disruption/replacement cassette for transformation.

### Example 5: Assembly of Folic Acid Operon Constructs for Transformation

After assembly of folic acid operon (see Example 3) DNA fragments with folate biosynthetic genes were further cut with Xbal restriction enzyme and ligated with synthetized DNA fragment for erythromycin resistance cassette (SEQ ID NO:19) with primers SEQ ID NO:40 and SEQ ID NO:41 (62° C., 40 s) and cut with Xbal to ensure compatible DNA ends for ligation. After ligation whole fragment was PCR amplified with primers (SEQ ID NO: 36 and SEQ ID NO: 39).

In the final step of assembly fragment (SEQ ID NO: 18) with lacA homology and regulatory promoter region was added. Fragments were cut with Spel restriction enzyme and used in ligation. Ligation mixture was used as PCR template with primers (SEQ ID NO: 42 and SEQ ID NO: 39), with which we finish assembly of artificial folate operon (shown in FIG. 4) as an expression cassette (SEQ ID NO: 20) for genome transformation into B. subtilis strains.

### Example 6: Selection of Possible Bacillus subtilis Host Strains for Engineering of Folate Production

Different Bacillus strains can be used as starting strains for engineering of folate production (Table 3). Bacillus strains can be isolated from nature or obtained from culture collections. Among others, starting strains for folate production can be selected among Bacillus subtilis strains that have already been subjected to classical methods of mutagenesis and selection in order to overproduce metabolites related to the purine biosynthetic pathway. For example, strains overproducing riboflavin, inosine and guanosine may be selected. Strains subjected to random mutagenesis and toxic metabolic inhibitors from purine and riboflavin pathway are preferred and are included in Table 3.

TABLE 3
Potential non-GMO starting strains of B. subtilis that
could be used for development of folate production.

| | | Alternative | | | |
|---|---|---|---|---|---|
| Species | Name | name | Product | Availability | Remarks |
| B. subtilis | 168 | ATCC6051 | none | yes | Type strain |
| B. subtilis | W23 | ATCC 23059/ | none | yes | Type strain |
| subsp. | | NRRL B-14472 | | | |
| spizizenii | | | | | |
| B. subtilis | RB50 | NRRL B18502 | riboflavin | yes | Developed by |
| | | | | | Roche/DSM |
| B. subtilis | RB58 | ATCC55053 | riboflavin | yes | Containing |
| | | | | | additional copy of |
| | | | | | rib operon |
| B. subtilis | VNII | VKPM B2116, | riboflavin | yes | Developed by VNII |
| | Genetika 304 | VBB38 | | | Genetika |
| B. subtilis | FERM-P | | riboflavin | no | Ajinomoto |
| | 1657 | | | | |
| B. subtilis | FERM-P | | riboflavin | no | Ajinomoto |
| | 2292 | | | | |
| B. subtilis | AJ12644 | FERM BP-3855 | riboflavin | no | Ajinomoto |
| B. subtilis | AJ12643 | FERM BP-3856 | riboflavin | no | Ajinomoto |
| B. subtilis | ATCC13952 | | inosine | yes | |
| B. subtilis | ATCC19221 | IFO 14123 | guanosine | yes | |
| B. subtilis | ATCC13956 | IFO 14124 | inosine | yes | |

VKPM B2116 strain is a hybrid strain of B. subtilis 168 strain (most common B. subtilis host strain with approx. 4 Mbp genome) with a 6.4 kbp island of DNA from the strain B. subtilis W23. Such architecture is common for most B. subtilis industrial strains and was obtained by transforming the 168 strain (tryptophan auxotroph trpC−) with W23 (prototrophic TrpC+) DNA. It has a 6.4 kbp W23 island in the genome, which is the same as in the commonly used strain B. subtilis SMY, which is one of the B. subtilis legacy strains with genome publicly available (Ziegler et al., The origins of 168, W23 and other Bacillus subtilis legacy strains, Journal of Bacteriology, 2008, 21, 6983-6995). VKPM B2116 strain is a direct descendant of the SMY strain, obtained by classical mutagenesis and selection. Another name for this strain is B. subtilis VNII Genetika 304. The description of construction of the strain in described in Soviet Union patent SU908092, filed in 1980. The mutations were obtained by subsequent mutagenesis and selection on metabolic inhibitors. The strain VKPM B2116 is resistant to roseoflavin, a toxic analogue of vitamin B2, due to a mutation in the ribC gene, encoding a flavin kinase. This strain is also resistant to 8-azaguanine, toxic analogue of purine bases.

### Example 7: Replacement of folC and Generation of the Optimum Host Strain for Folic Acid Production

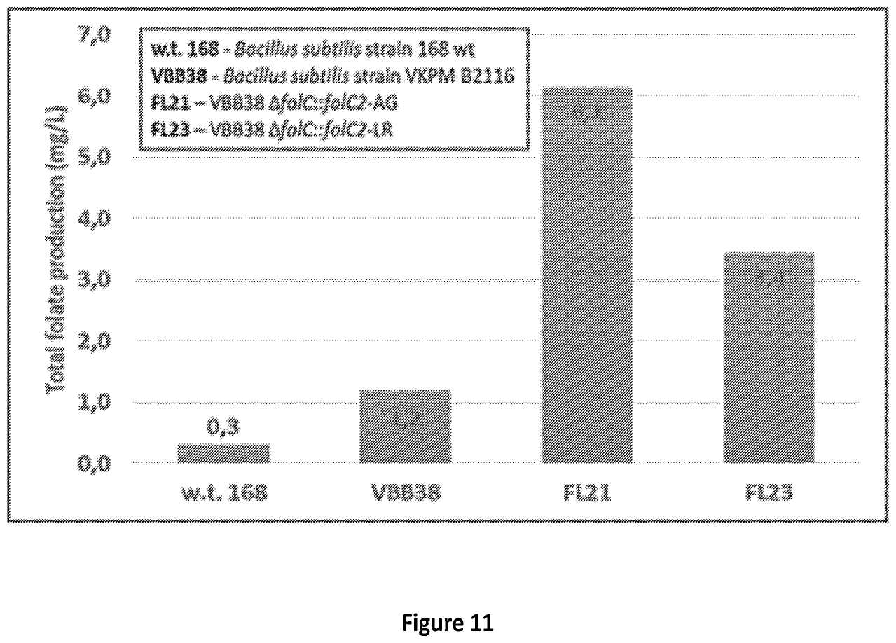

After construction of heterologous folC2 (folC2-AG or folC2-LR) gene expression cassette (see example 4 and FIG. 5) we have performed transformation of B. subtilis VBB38 and B. subtilis VBB38Δrib. Expression cassette with homologies for native folC gene disruption, was amplified by PCR with primers SEQ ID NO: 43 and SEQ ID NO: 44. After transformation colonies resistant to tetracycline were selected and native folC gene replacement, by a heterologous folC2 gene (A. gossypii or L. reuteri), was genetically confirmed with cPCR and sequencing of obtained PCR product. New strains were used to test the production yields of the total folates (see FIG. 11), and to compare the distribution of the total folates between the supernatant and the cell biomass.

### Example 8: Transformation of Bacillus subtilis

i) Bacillus subtilis Natural Competence Transformation

10 mL of SpC medium is inoculated from fresh plate of B. subtilis and cultured overnight. 1.3 mL of overnight culture is diluted into 10 mL of fresh SpC medium (9× dilution). OD450 is measured and is expected to be around 0.5. Cultures are grown for 3 h min at 37° C. 220 RPM. OD450 is measured again and is expected to be between 1.2-1.6. Cultures are diluted 1:1 with SpII (starvation medium). 3.5 ml of culture is mixed with 3.5 ml of starvation medium and tryptophan in concentration 50 ug/ml is added. Cultures are grown for additional 2 h at 37° C., 220 RPM. After incubation cultures are maximally competent for 1 h. 500 uL of competent cells is mixed with DNA (5-20 uL, depending on concentration) in 2 mL Eppendorf tube and incubated for 30 min at 37° C. with shaking. 300 uL of fresh LB is added for the recovery of competent cells and incubated for additional 30 min at 37° C. Eppendorf tubes are centrifuged at 3000 RPM, 5 min. Pellet is resuspended and plated on LB plates with appropriate antibiotic.

Medium:

10× T-Base

150 mM ammonium sulfate

800 mM K₂HPO₄

440 mM KH₂PO₄

35 mM sodium citrate

SpC (Minimal Culture Media)

100 mL 1× T-base

1 mL 50% glucose

1.5 mL 1.2% MgSO₄

2 ml 10% yeast extract

2.5 ml 1% casamino acids

SpII (Starvation Media)

100 ml 1× T-base

1 ml 50% glucose

7 ml 1.2% MgSO₄

1 ml 10% yeast extract

1 ml 1% casamino acids

0.5 ml 100 mM CaCl₂

### Example 9: Determination of Folate Operon Copy Number Using qPCR

We used real time quantitative PCR (qPCR) technique for determination of the number of copies of the integrated B. subtilis artificial folate operon genes. The copy numbers of the genes folP, folK, folE, dfrA and KnR (the gene for kanamycin resistance) in the artificial folate operon in the folate-producing B. subtilis transformants was estimated by (qPCR) with SYBR Green I detection. The copy number of the gene for kanamycin resistance (KnR) and the copy number of the folate biosynthesis genes folP, folK, folE, dfrA on artificial B. subtilis folate operon were quantified by qPCR. Genomic DNA of the B. subtilis strains was isolated with SW Wizard Genomic DNA Purification Kit (Promega). The concentration and purity of gDNA were evaluated spectrophotometrically at OD260 and OD280. The amount of gDNA used in all experiments was equal to the amount of gDNA of the reference strain. A B. subtilis with a single copy of artificial folate operon containing the genes folP, folK, folE, dfrA and KnR was used as a reference strain for relative quantification of the gene copy numbers. A housekeeping gene DxS, a single-copy gene in the B. subtilis genome, was used as the endogenous control gene. Quantification of gene copy number for the folate biosynthesis genes was performed using specific set of primers (primer pair SEQ ID NO:59 and SEQ ID NO:60 for folP gene, primer pair SEQ ID NO:61 and SEQ ID NO:62 for folK gene, primer pair SEQ ID NO:63 and SEQ ID NO:64 for folE gene, primer pair SEQ ID NO:65 and SEQ ID NO:66 for dfrA gene) for quantification of kanamycin resistance marker attached to folate operon (primer pair SEQ ID NO:67 and SEQ ID NO:68) and for reference DxS gene primer pair SEQ ID NO:71 and SEQ ID NO:72 were used. The qPCR analysis was run on StepOne™ Real-Time PCR System and quantification was performed by using the 2^{-ΔΔCT} method.

The gene copy numbers of the genes in the artificial BS-FOL-OP strains were quantified relatively to the strain with one copy of the genes. The KnR gene of the B. subtilis strain with one copy number was used as the reference strain for relative quantification of the gene copy numbers of genes in the artificial folate operon in B. subtilis transformed strains. The qPCR relative quantification of the genes folP, folK, folE, dfrA and KnR genes showed 6-fold increase in RQ values compared to B. subtilis strain with single copy genes. Folate overproducing strains FL179 and FL722 were confirmed to have multi-copy integration of folic acid synthetic operon.

### Example 10: Cultivation of Bacillus subtilis Strains

Serial dilutions from frozen cryovial are made and plated on to MB plates with appropriate antibiotic and incubated for approximately 48 h at 37° C. For further testing at least 10-20 single colonies from MB plates use for each strain. First re-patch 10-20 single colonies on fresh MB plates (with the same concentration of antibiotics) for testing.

For vegetative stage MC medium is used and inoculated with 1 plug per falcon tube (or 5 plugs per baffled Erlenmeyer flask or small portion of patch for microtiter plates). Appropriate antibiotics are added into medium. For microtiter plates 500 ul of medium is used in 96 deep well, for falcon tubes is used 5 ml of medium (in 50 ml falcon tube) and for Erlenmeyer flask 25 ml (in 250 ml flask). Cultures are incubated at 37° C. for 18-20 h at 220 RPM.

Inoculation into production medium (MD) is after 18-20 h in vegetative medium. 10% inoculum is used (50 ul for MW, 0.5 ml for falcon tube and 2.5 ml Erlenmeyer flask). Each strain is tested in two aliquots. For microtiter plates 500 ul of medium is used in 48 deep well, for falcon tubes is used 5 ml of medium and for baffled Erlenmeyer flask 25 ml. Wires are used in falcon tubes for better aeration, as are gauzes used instead of the stoppers on Erlenmeyer flasks. Cultures are incubated at 37° C. for 48 h at 220 RPM. After 24 and 48 hours titer of total folates was measured using the microbiological assay, according to the developed procedures

Best candidate strains are retested in the same manner and after several confirmations prepared for testing in bioreactors. 100 ul of frozen culture of selected strain for bioreactor testing is spread on to MB plates with appropriate antibiotic and incubated for approximately 48 h at 37° C. Complete biomass is collected with 2 ml of sterile 20% glycerol per plate. Collected biomass is distributed into 100 ul aliquots and frozen at −80° C. This is used as working cell bank for bioreactor testing.

Medium Composition:

1) MB (Plates)

Trypton 10 g/l

Yeast extract 5 g/l

NaCl 5 g/l

Maltose 20 g/l

Agar 20 g/l

pH 7.2-7.4

Autoclaved 30 min, 121° C.

After autoclaving and cooling down appropriate antibiotics are added.

2) MC (Vegetative Medium)

Molasses 20 g/l

CSL 20 g/l

Yeast extract 5 g/l

MgSO₄*7H₂O 0.5 g/l

(NH₄)₂SO₄ 5 g/l

Ingredients are mixed together and pH set to 7.2-7.4. KH₂PO₄—K₂HPO₄ solution is then added in final concentration for KH₂PO₄ 1.5 g/l and K₂HPO₄ 3.5 g/l. Medium is distributed into falcon tubes (5 ml/50 ml-falcon tubes) or Erlenmeyer flasks (25 ml/250 ml-baffled Erlenmeyer flask) and autoclaved 30 min, 121° C. Sterile glucose is added after autoclaving in final concentration 7.5 g/l. Antibiotics are added prior to inoculation.

3) MD (Production Medium)

Yeast 20 g/l

Corn steep liquor (CSL) 5 g/l

MgSO₄*7H₂O 0.5 g/l

para-aminobenzoic acid (pABA) 0.5 g/L

Ingredients are mixed together and pH set to 7.2-7.4. KH₂PO₄—K₂HPO₄ solution is then added in final concentration for KH₂PO₄ 1.5 g/l and K₂HPO₄ 3.5 g/l The medium is autoclaved at 121° C. for 30 min. Sterile urea solution (20 ml of stock solution, final concentration is 6 g/L), sterile glucose solution (250 ml of stock solution, final concentration is 100 g/L glucose), sterile pABA solution (100 ml of stock solution, final concentration is 0.5 g/L) and 150 ml of sterile water are added after autoclaving to obtain 1 L of MD+pABA500 medium. Appropriate antibiotics were added prior to inoculation. Medium is then distributed into sterile Erlenmeyer flasks (25 ml/250 ml-baffled Erlenmeyer flask.

### Example 11: Microbiological Assay for Quantification of Total Folates in Fermentation Broths

A microbiological assay using Enterococcus hirae NRRL B-1295 was used for detection of the total folates produced in the strains of Bacillus subtilis. The microbiological assay was used for the evaluation of the intracellular (retained in the biomass) and extracellular (released into the culture medium) total folates produced by B. subtilis. For the microbiological assay, the indicator organism Enterococcus hirae NRRL B-1295 is used, which is auxotrophic for folates or folic acid. E. hirae is precultured in the rich growth medium, containing folates (Lactobacilli AOAC broth) at 37° C. for 18-24 h. It is then washed in the growth medium without folates (folic acid assay medium) to remove the residual folates. The washed E. hirae culture is inoculated into the assay medium without folic acid. The microbiological assay is set up in 96-well microtiter plates. Appropriately diluted media samples to be assayed and the standard solutions of folic acid are added to the growth medium containing the indicator strain, and the plate is incubated at 37° C. for 20 h. The growth response of the indicator organism is proportional to the amount of folic acid/folates present in the media samples/controls. The standard curve is constructed for each assay by adding a set of standard solutions of folic acid to the growth medium and the indicator strain. The growth is measured by measuring the optical density (OD) at 600 nm wavelength. The growth response of E. hirae to the test samples is compared quantitatively to that of the known standard solutions. A dilution series containing various concentrations of folic acid is prepared and assayed as described above. The standard curve is obtained by plotting the measured OD₆₀₀ at known concentrations of folic acid. The standard curve is used to calculate the amounts of total folates in the test samples. The indicator organism E. hirae NRRL B-1295 is used to detect the concentrations of total folates in the range from 0.05 to 0.7 ng/mL in the measured sample. The total extracellular and intracellular folates produced by B. subtilis strains can be estimated by adding appropriately diluted test samples to the indicator organism E. hirae in folic acid assay medium.

### Example 12: Analysis of Total Folate Yields of Different Starting Strains and Initial folC-Replaced and Folic Acid Operon Amplified Strains

The transformants in which folC gene was replaced by a heterologous folC2 gene from either A. gossypii (B. subtilis strain FL21) or L. reuteri (B. subtilis strain FL23) and transformants with amplified folic acid operon were tested for total folate amounts at the shaker scale (5 ml production medium MD). After the fermentation, the samples of the fermentation broth (200 μl) was carefully collected to obtain a homogeneous sample and diluted 10 times in the ice-cold extraction buffer (0.1 M phosphate buffer with 1% (w/v) ascorbic acid). The samples were centrifuged at 14,000 rpm and 4° C. for 10 min and filter-sterilized (0.22 um pore size). For the microbiological assay samples were serially diluted in the extraction buffer and kept at 4° C. until the microbiological assay was set up. In the Table 4 results for selected strains measured by the microbiological assay are presented.

TABLE 4
Total folate production of different Bacillus subtills
strains in experiments at shaker scale (5 ml)

| | Total folate |
|---|---|
| | production |
| Description of strain | (mg/L) |
| B. subtilis w.t. 168 | 0.31 |
| VBB38 (B. subtilis VKPM B2116) | 1.24 |
| FL23 (B. subtilis VBB38 folC::tetR P-veg/folC2-LR) | 3.4 |
| FL1027 (B. subtilis VBB38 folC::tetR P-veg/folC2-LR, | 238.0 |
| lacA::ermAM P-15/FOL-OP-BS2) | |
| FL21 (B. subtilis VBB38 folC::tetR P-veg/folC2-AG) | 6.1 |
| FL260 (B. subtilis VBB38 folC::tetR P-veg/folC2-AG; | 45.7 |
| amyE::cat P-15/FOL-OP-AG) | |
| FL84 (B. subtilis VBB38 folC::tetR P-veg/folC2-AG; | 55.2 |
| amyE::cat P-15/FOL-OP-LL) | |
| FL179 (B. subtilis VBB38 folC::tetR P-veg/folC2-AG; | 573.0 |
| amyE::kanR P-veg/FOL-OP-BS1) | |
| FL722 (B. subtilis VBB38 folC::tetR P-veg/folC2-AG; | 587.4 |
| lacA::ermAM P-15/FOL-OP-BS2) | |

### Example 13: Determination of Concentrations Folate Forms and Related Compounds Using LC-MS and Identification of 10-Formyl-Dihydrofolic Acid and 10-Formyl Folic Acid as Two Main Products

In addition to the microbiological assay, our aim was to develop sensitive and versatile analytical method, with reasonably short analytical run time. The method had to be LCMS compatible with volatile mobile phase, and also had to enable UV detection and give good chromatographic separation of as many folate-related analytes as possible.

Instruments and Materials:

The method was developed on Thermo Accela 1250 HPLC instrument with PDA detector, coupled with MS/MS capable mass spectrometer Thermo TSQ Quantum Access MAX, equipped with hESI source. Method has been set-up on Thermo Acclaim RSLC PA2, 150×2.1 mm HPLC column with 2.2 μm particle size. PDA detector is set at 282 nm, with bandwidth 9 nm and 80 Hz scan rate, and also DAD scan from 200-800 nm. Column oven is set at 60° C. and tray cooling at 12° C. Injection solvent is 10% methanol in water, with wash and flush volume: 2000 μl. Injection volume is set at 10 μl and can also be set at 1 μl when higher concentrations of analytes are expected. Mobile phase A is 650 mM acetic acid in water, and mobile phase B is methanol. Mobile phase flow is 0.5 ml/min and total run time is 20 min. Method is using gradient program in Table 5 and MS spectrometer parameters described in Table 6.

TABLE 5
Gradient program for the chromatographic analysis

| Time/min | % A | % B |
|---|---|---|
| 0.00 | 100 | 0 |
| 2.00 | 100 | 0 |
| 16.00 | 82 | 18 |
| 16.01 | 100 | 0 |
| 20.00 | 100 | 0 |

TABLE 6
MS spectrometer tune parameters and
other MS/MS relevant parameters.

| Tune parameters: | | | Other method parameters: | |
|---|---|---|---|---|
| Ionization | hESI+ | | Polarity: | Positive |
| Spray voltage | 4000 | V | Scan width | 5.000 |
| | | | (m/z): | |
| Vaporizer temperature | 350° | C. | Scan time (s): | 0.100 |
| Sheath gas pressure | 55 | | Q1 (FWHM): | 0.70 |
| Aux gas pressure | 5 | | Micro scans: | 1 |
| Capillary temperature | 300° | C. | Data type: | Centroid |
| Tube lens offset | SRM table | | Chrom filter | none |
| | | | P.W. | |
| Skimmer offset | 0 | | MS Acquire | 15 |
| | | | time (min): | |
| Collision pressure | 1.0 | torr | Divert valve: | none |

LCMS detector is coupled after DAD detector, and analytes are observed in scan from 400-600 m/z mode, in SIM mode at their M.W.+1 and MS/MS mode (Table 6). Standards were prepared with weighting and dissolving in 0.1 M NaOH solution (Table 7 and Table 8) and immediately put to HPLC instrument.

TABLE 7
Available standards.

| Analyte: | Purity: | Source: | Abbreviation: |
|---|---|---|---|
| Folic acid | 91.3% | Pharmacopoeia | FA |
| Dihydro folic acid | >80.0% | Sigma | DHF |
| Tetrahydro folate | >65.5% | Sigma | THF |
| 5-methyl tetrahydro | >81.0% | Carbosynth | 5M-THF, |
| folate | | | 5-methyl THF |
| 10-formyl folic acid | 91.4% | EDQM | 10F-FA, |
| | | | 10-formyl FA |
| 5-formyl tetrahydro | >90.0% | EDQM | 5F-THF, |
| folate | | | 5-formyl THF |

TABLE 8
Observed standards and their related MS/MS method settings.

| | Parent | Product(s) | Collision | Tube | Retention time |
|---|---|---|---|---|---|
| Analyte: | m/z: | m/z: | E: | lens: | (+/−0.1 min) |
| Folic acid | 442 | 295 | 19 | 50 | 10.2 min |
| Dihydro f.a. | 444 | 178, 297 | 19 | 50 | 10.4 min |
| Tetrahydro f.a. | 446 | 299, 318, 361, 387 | 20 | 50 | 6.1 min |
| 5-methyl THF | 460 | 180, 314 | 20 | 50 | 7.0 min |
| 10-formyl f.a. | 470 | 295, 323 | 20 | 50 | 12.1 min |
| 10-formyl dihydro f.a. | 472 | 297, 325 | 20 | 50 | 10.6 min |
| 5-formyl THF | 474 | 299, 327 | 20 | 50 | 12.6 min |

Method has linear response for MS/MS detection up to 1000 mg/L of analyte, with correlations above 90% for all standards.

### Example 14: Different Ratio of Folic Acid and Derivatives Production Through Genetically Modified Bacillus subtilis

The transformants in which folC gene was replaced by a heterologous folC2 gene from either A. gossypii (B. subtilis strain FL21) or L. reuteri (B. subtilis strain FL23) and transformants with amplified folic acid operon were tested for total folate amounts at the shaker scale (5 ml production medium MD).

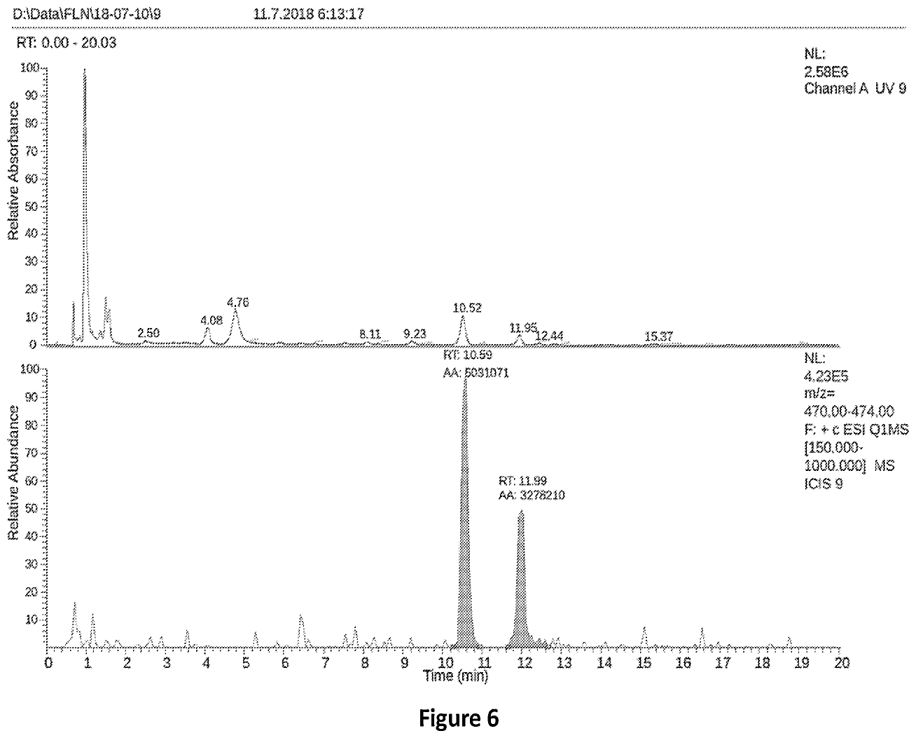

The strains were patched on MB plates with appropriate antibiotics and incubated at 37° C. for 2 days. For shake-flasks experiments, the grown strains were transferred to 5 ml of MC (seed) medium in Falcon 50 mL conical centrifuge tubes (1 plug/5 ml) and cultivated on a rotary shaker at 220 RPM and 37° C. for 16-18 h. A 10-% inoculum of the seed culture was used to inoculate 5 mL of the production medium (MD+pABA500). The strains were cultivated on a rotary shaker at 220 RPM and 37° C. for 48 h in the dark. After the fermentation, the samples of the fermentation broth (200 μl) was carefully collected to obtain a homogeneous sample and diluted 10 times in the ice-cold extraction buffer (0.1 M phosphate buffer with 1% (w/v) ascorbic acid). The samples were centrifuged at 14,000 rpm and 4° C. for 10 min and filter-sterilized (0.22 um pore size). For the quantification of different folate species HPLC method was used as described in Example 13. Results of different B. subtilis strain are shown in Table 9 and representative HPLC chromatogram of fermentation broth sample is shown in FIG. 6.

TABLE 9
Total folate production of different Bacillus subtills
strains in experiments at shaker scale (5 ml)

| | | 5M-THF | FA | 10F-DHF | 10F-FA |
|---|---|---|---|---|---|
| Strain | Strain description | (mg/L) | (mg/L) | (mg/L) | (mg/L) |
| wt 168 | B. subtillis wild type | 0.16 | 0.15 | 0.01 | 0.86 |
| VBB38 | B. subtilis VKPM B2116 | 0.35 | 0.01 | 0.02 | 0.09 |
| FL 23 | VBB38 folC::tetR P-veg/folC2-LR | 1.11 | 0.01 | 0.01 | 0.03 |
| FL 21 | VBB38 folC::tetR P-veg/folC2-AG | 18.20 | 0.01 | 0.03 | 2.64 |
| FL 84 | FL21 amyE::cat P-15/FOL-OP-LL | 25.46 | 0.02 | 0.34 | 18.81 |
| FL 260 | FL21 amyE::cat P-15/FOL-OP-AG | 21.67 | 0.04 | 1.52 | 44.50 |
| FL 179 | FL21 amyE::kanR P-veg/FOL-OP-BS1 | 97.05 | 0.03 | 16.22 | 373.22 |
| FL 722 | FL21 lacA::ermAM P-15/FOL-OP-BS2 | 20.21 | 0.30 | 13.21 | 351.00 |

Strain FL179 with heterologous folC-AG and overexpressed folate biosynthetic genes from B. subtilis showed 43297% increased 10-formyl folic acid production compared to the wild type strain Bacillus subtilis 168.

### Example 15: Oxidative Conversion of 10-Formyldihydrofolic Acid to 10-Formyl Folic Acid

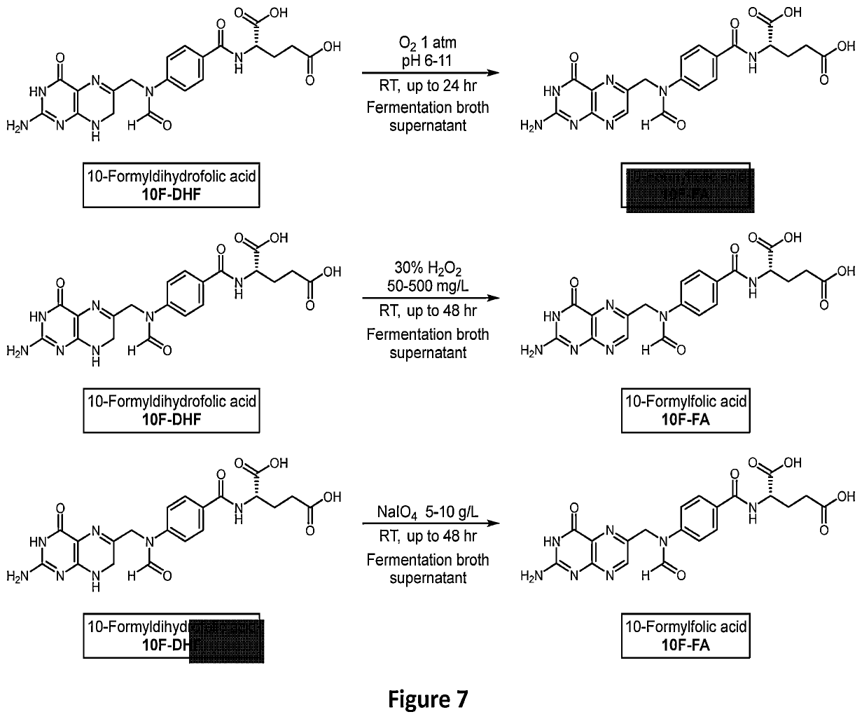

At the end of the fermentation, HPLC analysis of broth detected a relatively high amount (85 Area %) of 10-formyldihydrofolic acid (10F-DHF). Furthermore, we observed that 10-formyldihydrofolic acid can be oxidatively converted to 10-formylfolic acid (see FIG. 7). Accordingly, we started to develop a protocol, which will provide a quantitative conversion to 10-formylfolic acid. We anticipate the subsequent deformylation step will provide a folic acid in the highest possible yield. Literature search revealed a report describing the oxidation of tetrahydrofolic acid by air in aqueous solutions at specific pH values (Reed1980). Based on this report, at pH values 4, 7 and 10 the major products of oxidation are p-aminobenzoylglutamic acid (PABG) and 6-formylpterin. In addition, 7,8-dihydrofolate intermediate was only detected at pH=10. We carried out the series of oxidation experiments on the fermentation broth supernatant to facilitate a swift conversion of 10-formyldihydrofolic acid to 10-formylfolic acid. We examined several oxidation reagents such as O₂, H₂O₂ and NaIO₄ (see FIG. 7).

TABLE 10
Effect of pH on oxidation of 10-formyldihydrofolic acid to
10-formylfolic acid in the fermentation broth supernatant
with oxygen.

| | | | | | | | | SUM |
|---|---|---|---|---|---|---|---|---|
| | | | | | 10F-DHF | 10F-FA | FA | FOL |
| exp | pH | oxidant | time | temp | mg/L | mg/L | mg/L | mg/L |
| 1 | 7 | — | 0 hr | 25° C. | 782.9 | 180.2 | 12.4 | 975.5 |
| 2 | 6 | O₂ 1 atm | 48 hr | 25° C. | 140.0 | 368.2 | 7.3 | 515.5 |
| 3 | 7 | O₂ 1 atm | 48 hr | 25° C. | 253.5 | 366.1 | 10.3 | 611.9 |
| 4 | 8 | O₂ 1 atm | 48 hr | 25° C. | 268.1 | 376.5 | 12.0 | 656.5 |
| 5 | 9 | O₂ 1 atm | 48 hr | 25° C. | 199.4 | 293.6 | 0 | 493 |
| 6 | 10 | O₂ 1 atm | 48 hr | 25° C. | 80.7 | 288.4 | 0 | 369.1 |
| 7 | 11 | O₂ 1 atm | 48 hr | 25° C. | 0 | 351.3 | 0 | 351.3 |

Experiments were conducted in 50 mL round bottom flasks using 10 mL of the fermentation broth supernatant. pH values were set by 1.0 M and 0.1 M NaOH solution. Progress of reaction and results were measured by HPLC. The HPLC samples were prepared in the extraction buffer (0.1 M phosphate buffer with 1% (w/v) ascorbic acid). All reactions were stirred protected from the light for 48 hours at ambient temperature (25° C.).

Required pH values were adjusted with 1 M and 0.1 M HCl or NaOH. Reactions at lower pH values are slower and maintain relatively high sum of folates (Table 10, entries 2-4). On the contrary, reactions at higher pH values (Table 10, entries 5-7) improve the consumption of 10-formyldihydrofolic acid albeit significantly reduce the sum of the folates. We anticipate we could use alternative reagents for oxidation such as hydrogen peroxide or sodium periodate.

Representative Experimental Procedure:

Fermentation broth was centrifuged at 4,500 rpm and the supernatant decanted. The 10 mL of fermentation broth supernatant was pipetted into the 50 mL round bottom flasks equipped with stirring bars, pH meter and aluminum foil for light protection. Sodium hydroxide or hydrochloric acid (1.0 M and 0.1 M for fine tuning) was added dropwise to set the pH value and reaction was stirred vigorously for 24 hours under the ambient temperature (25° C.). The reaction mixture was purged with an air from the balloon. After 48 hours of stirring, 1 mL of each fermentation broth was diluted in duplicates with 9 mL of extraction buffer (0.1 M phosphate buffer with 1% (w/v) ascorbic acid). The suspensions were stirred on vortex, centrifuged at 4,500 rpm, filtered through 0.22 μm filter and analyzed on HPLC.

TABLE 11
Effect of hydrogen peroxide concentration on oxidation
of 10-formyl-dihydrofolic acid to 10-formylfolic
acid in the fermentation broth supernatant.

| | | | | | | | | SUM |
|---|---|---|---|---|---|---|---|---|
| | | mg/ | | | 10F-DHF | 10F-FA | FA | FOL |
| exp | oxidant | L | time | temp | mg/L | mg/L | mg/L | mg/L |
| 1 | — | 0 | 0 hr | 25° C. | 735.5 | 137.4 | 0 | 872.9 |
| 2 | H₂O₂ | 50 | 24 hr | 25° C. | 390.8 | 299.5 | 12 | 702.4 |
| 3 | H₂O₂ | 100 | 24 hr | 25° C. | 397.3 | 308.7 | 24.2 | 730.2 |
| 4 | H₂O₂ | 250 | 24 hr | 25° C. | 355 | 325.4 | 12.7 | 693.1 |
| 5 | H₂O₂ | 500 | 24 hr | 25° C. | 383.5 | 315.5 | 12.9 | 711.9 |
| 6 | H₂O₂ | 50 | 48 hr | 25° C. | 193.5 | 354.7 | 0 | 548.1 |
| 7 | H₂O₂ | 100 | 48 hr | 25° C. | 183.4 | 529.5 | 0 | 712.8 |
| 8 | H₂O₂ | 250 | 48 hr | 25° C. | 185.9 | 534.3 | 0 | 720.1 |
| 9 | H₂O₂ | 500 | 48 hr | 25° C. | 174.4 | 539.6 | 0 | 714.0 |

Experiments were conducted in 50 mL round bottom flasks using 10 mL of the fermentation broth supernatant. Hydrogen peroxide was added dropwise as 30% solution in water. Progress of reaction and results were measured by HPLC. The HPLC samples were prepared in the extraction buffer (0.1 M phosphate buffer with 1% (w/v) ascorbic acid). All reactions were stirred protected from the light for 48 hours at ambient temperature (25° C.).

Hydrogen peroxide, an alternative oxidant for the oxidative conversion of 10-formyldihydrofolic acid to 10-formylfolic acid was added in concentration range from 50-500 mg/L thus providing more advanced results (Table 11). During the first 24 hours of reaction, the concentration of 10-formyldihydrofolic acid dropped to 50% of its initial value. Prolongation of reaction to 48 hours provided a good conversion thus maintaining a relatively high sum of total folates.

Representative Experimental Procedure:

Fermentation broth was centrifuged at 4,500 rpm and the supernatant decanted. The 10 mL of fermentation broth supernatant was pipetted into the 50 mL round bottom flasks equipped with stirring bars, pH meter and aluminum foil for light protection. Hydrogen peroxide was added dropwise as 30% solution in water and the reaction mixture stirred vigorously for 24-48 hours under the ambient temperature (25° C.). After 48 hours of stirring, 1 mL of each fermentation broth was diluted in duplicates with 9 mL of extraction buffer (0.1 M phosphate buffer with 1% (w/v) ascorbic acid). The suspensions were stirred on vortex, centrifuged at 4,500 rpm, filtered through 0.22 μm filter and analyzed on HPLC.

TABLE 12
Effect of sodium periodate concentration on oxidation
of 10-formyl-dihydrofolic acid to
10-formylfolic acid in the fermentation broth supernatant.

| | | | | | 10F- | | | SUM |
|---|---|---|---|---|---|---|---|---|
| | | mg/ | | | DHF | 10F-FA | FA | FOL |
| exp | oxidant | L | time | temp | mg/L | mg/L | mg/L | mg/L |
| 1 | — | 0 | 0 hr | 25° C. | 735.5 | 137.4 | 0 | 872.9 |
| 2 | NaIO₄ | 5 | 24 hr | 25° C. | 278.5 | 326.5 | 34.1 | 639.1 |
| 3 | NaIO₄ | 5 | 48 hr | 25° C. | 111.8 | 376.7 | 40.3 | 528.8 |
| 4 | NaIO₄ | 10 | 24 hr | 25° C. | 84.6 | 449.6 | 212.6 | 746.8 |
| 5 | NaIO₄ | 10 | 48 hr | 25° C. | 0 | 575.6 | 251.1 | 826.7 |

Experiments were conducted in 50 mL round bottom flasks using 10 mL of the fermentation broth supernatant. Sodium periodate was added in a single portion. Progress of reaction and results were measured by HPLC. The HPLC samples were prepared in the extraction buffer (0.1 M phosphate buffer with 1% (w/v) ascorbic acid). All reactions were stirred protected from the light for 48 hours at ambient temperature (25° C.).

Sodium periodate is often used as the reagent of choice for capricious substrates. Our initial experimentation with this reagent revealed that the effective concentration for the oxidative conversion is between 1-10 g/L. Sodium periodate was added in two different concentrations, 5 g/L and 10 g/L. During the first 24 hours of reaction, the concertation of 10-formyldihydrofolic acid dropped significantly from its initial value (Table 12). Prolongation of reaction to 48 hours provided an excellent conversion thus maintaining a relatively high sum of total folates.

Representative Experimental Procedure:

Fermentation broth was centrifuged at 4,500 rpm and the supernatant decanted. The 10 mL of fermentation broth supernatant was pipetted into the 50 mL round bottom flasks equipped with stirring bars, pH meter and aluminum foil for light protection. Sodium periodate was added in a single portion and the reaction mixture stirred vigorously for 24 hours under the ambient temperature (25° C.). After 48 hours of stirring, 1 mL of each fermentation broth was diluted in duplicates with 9 mL of extraction buffer (0.1 M phosphate buffer with 1% (w/v) ascorbic acid). The suspensions were stirred on vortex, centrifuged at 4,500 rpm, filtered through 0.22 μm filter and analyzed on HPLC.

### Example 16: Production of Folates in 5 L Bioreactor Volume

The production of folates can be greatly improved in bioreactors where appropriate conditions are used for the cultivation and production of folates. The process includes the preparation of the pre-culture and the main fed-batch bioprocess.

i) Preparation of the Pre-Culture

The pre-culture medium (FOL-MC, Table 13) in flasks is seeded with the working cell bank of strain FL179 and cultivated on a rotary shaker at 37° C. and 220 RPM (2″ throw) for 11-14 hours.

ii) Fed-Batch Bioprocess

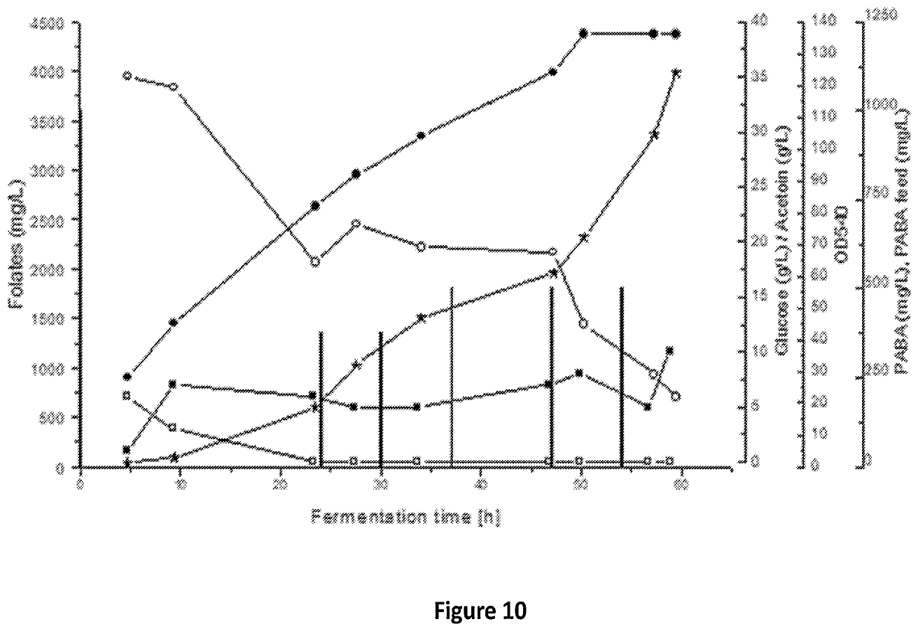

The production of folates is carried out in a 5 L bioreactor using the FOL-ME medium (Table 14). The bioreactor starting parameters are Agitation=600 RPM, Aeration=1 vvm, pH is controlled at 7 using ammonium hydroxide solution. The bioreactor is inoculated with 10% of the pre-culture. The DO is controlled by agitation and airflow to keep the air saturation above 30%. When glucose in the fermentation broth is depleted, feeding of a glucose and CSL mixture (Table 15) is started. The rate of feed addition needs to be carefully controlled and the feeding rate is controlled at a level, which does not lead to acetoin (not more than 10 g/L) accumulation. If no acetoin is detected in the fermentation broth the feeding rate is too low. para-aminobenzoic acid (PABA) concentration in the fermentation broth needs to be measured at regular intervals and kept above 500 mg/L by batch feeding of a concentrated PABA stock solution (50 g/L). The bioprocess is usually finished in 50 hours. Folates production bioprocess profile is shown in FIG. 10.

TABLE 13
FOL-MC pre-culture medium

| Component | Amount | |
|---|---|---|
| Molasses | 20 | g/L |
| Corn steep liquor (CSL) | 20 | g/L |
| Yeast | 5 | g/L |
| (NH4)2SO4 | 5 | g/L |
| MgSO4 × 7H2O | 0.5 | g/L |
| KH2PO4 | 1.5 | g/L |
| K2HPO4 | 3.5 | g/L |
| glucose | 7.5 | g/L |
| Kanamycin | 10 | mg/L |
| Tetracycline | 10 | mg/L |

TABLE 14
FOL-ME production medium

| Component | Amount | |
|---|---|---|
| Soybean powder | 25 | g/L |
| Corn steep liquor (CSL) | 40 | g/L |
| Yeast | 5 | g/L |
| (NH4)2SO4 | 4 | g/L |
| MgSO4 × 7H2O | 2.05 | g/L |
| KH2PO4 | 1.5 | g/L |
| K2HPO4 | 3.5 | g/L |
| Glucose | 30 | g/L |
| Kanamycin | 10 | mg/L |
| Tetracycline | 10 | mg/L |
| Sodium para-aminobenzoate | 1 | g/L |
| (PABA) | | |

TABLE 15
Feeding solution (glucose + CSL)

| Component | Amount |
|---|---|
| Glucose monohydrate | 400 g/L |
| Corn steep liquor (CSL) | 310 g/L |

### Example 17: Determination of Expression Levels of Folate Biosynthetic Genes Using qPCR

Culture growth conditions: B. subtilis culture was grown in LB medium to the exponential phase. The culture was mixed with 2 volumes of the RNA protect Bacteria Reagent (QIAGEN), centrifuged for 10 min at 4500 rpm and frozen at −80° C. or processed immediately. Cell pellet was resuspended in 200 μL of TE buffer containing 1 mg/mL lysozyme for 15 min in order to remove the cell wall. RNA was isolated by using QIAGEN Rneasy mini kit according to the manufacturer protocol. The obtained RNA was checked for concentration and quality spectrophotometrically. The isolated RNA was treated with DNase (Ambion kit) and reverse-transcribed to cDNA by using RevertAid H Minus First Strand cDNA Synthesis Kit (Thermo Scientific). The obtained cDNA was diluted and the final yield of cDNA is cca 2.5 ng/μL.

The obtained cDNA was analysed by qPCR analysis (StepOne Real-Time PCR System, Applied Biosystems) with SYBR Green I (Thermo Scientific) detection. The expression of the folate operon genes in the integrated B. subtilis artificial folate operon genes folP, folK, folE, dfrA was quantified by real time quantitative PCR (qPCR) technique.

Internal control gene used as reference for normalization of quantitative qPCR expression data, 16S rRNA gene from B. subtilis was used. The expression of the folate biosynthesis genes was determined using specific set of primers (primer pair SEQ ID NO:59 and SEQ ID NO:60 for folP gene, primer pair SEQ ID NO:61 and SEQ ID NO:62 for folK gene, primer pair SEQ ID NO:63 and SEQ ID NO:64 for folE gene, primer pair SEQ ID NO:65 and SEQ ID NO:66 for dfrA gene) and for 16S gene selected as internal control primer pair SEQ ID NO:69 and SEQ ID NO:70 were used. The qPCR analysis was run on StepOne™ Real-Time PCR System and quantification was performed by using the 2^{-ΔΔCT} method.

The best folate producing strain FL722 bearing multicopy of synthetic folate operons at two separate genome locations (amyE and lacA) was confirmed to have the strongest expression levels of folate biosynthetic genes.

### Example 18: Chemical Conversion of 10-Formyl Folic Acid to Folic Acid

Acid-Mediated Deformylation

Deformylation of 10-formylfolic acid was conducted on 0.01 mmol scale (5 mg). 10-formylfolic acid was weighed in the 2 mL Eppendorf tube equipped with a stirring bar and suspended in distilled water (1 mL). The suspension was treated with acid (50 equiv., 0.5 mmol) and allowed to stir for 16 hours at ambient temperature. Subsequently, a suspension (200 μL) was diluted with DMSO (800 μL), homogenized on the vortex stirrer and analyzed on HPLC. Results of deformylation are presented in Table 16.

TABLE 16
Effect of different acids on of N-deformylation of 10-formylfolic acid

| exp | solvent | acid | eq. | mmol | time | temp | mmol | conv. to FAª |
|---|---|---|---|---|---|---|---|---|
| 1 | H₂O | HC1 | 50 | 0.5 | 16 hr | 25° C. | 0.01 | 98.8% |
| 2 | H₂O | DOWEX | 50 | 0.5 | 16 hr | 25° C. | 0.01 | n.d.^{b} |
| 3 | H₂O | TFA^{c} | 50 | 0.5 | 16 hr | 25° C. | 0.01 | 92.9% |
| 4 | H₂O | TCA^{d} | 50 | 0.5 | 16 hr | 25° C. | 0.01 | 95.3% |
| 5 | H₂O | HCOOH | 50 | 0.5 | 16 hr | 25° C. | 0.01 | 1.1% |
| 6 | H₂O | PTSA^{e} | 50 | 0.5 | 16 hr | 25° C. | 0.01 | 97.8% |
| 7 | H₂O | CH₃COOH | 50 | 0.5 | 16 hr | 25° C. | 0.01 | 0.7% |
| 8 | H₂O | H₂SO₄ | 50 | 0.5 | 16 hr | 25° C. | 0.01 | 100% |

All experiments were conducted in 2 mL Eppendorf tubes using 10-formylfolic acid (5 mg, 0.01 mmol). ^{a}Conversion was measured by HPLC. ^{b}n.d.—not detected. Neither 10-formylfolic acid nor folic acid were detected in this experiment due to a probable adsorption of the analyte to Dowex 50WX2 resin. ^{c}TFA—Trifluoroacetic acid. ^{d}TCA—Trichloroacetic acid. ^{e}PTSA—p-Toluenesulfonic acid.

Deformylation of 10-formylfolic acid with strong inorganic acids proceeded almost quantitatively to folic acid (Table 16, entries 1 and 8). Alternatively, deformylation with stronger organic acids provided folic acid with nearly equal efficiency (Table 16, entries 3, 4 and 6). As expected, deformylation with formic and acetic acid provided no conversion (Table 16, entries 5 and 7). HPLC analysis of deformylation using Dowex 50WX2 resin provided no detection for a starting material nor product since analyte probably remained adsorbed to the resin and requires elution.

Acid-Mediated N-Deformylation of 10-Formylfolic Acid in the Fermentation Broth

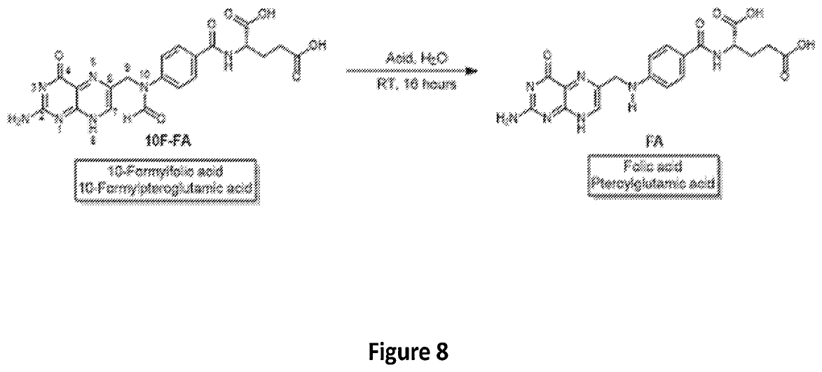

In previous experiments we have illustrated that deformylation of 10-formylfolic acid standard using a strong acid provided a clean conversion to folic acid shown in FIG. 8. Herein we applied the same principle on a more complex system, a fermentation broth. To continue experimenting on biological samples, we have selected a hydrochloric acid (HCl) as a deformylation reagent since it is highly effective and less expensive than other acids we studied. HPLC analysis of fermentation broth from Example 16 showed a substantial amount of 10-formylfolic acid among other folates formed during a biosynthesis (10-formylfolic acid 46% Area; 5-imidomethyltetrahydrofolic acid 47% Area and 5-methyltetrahydrofolic acid 7% Area). Samples of fermentation broth were treated with 1 M HCl up to different pH levels (pH=4, 3, 2, 1 and 0) and stirred for 24 hours at ambient temperature (25° C.) protected from light. According to our HPLC assay, only at lower pH levels (pH=1 and 0) deformylation provided a modest amount of folic acid. Based on these results, we are confident that acid-mediated deformylation strategy is potentially applicable during downstream processing of folic acid. In order to develop a cost-effective deformylation protocol of formyl folate species in a complex system such as fermentation broth, further optimization of acid amount and reaction temperature is essential.

Well-stirred fermentation broth from Example 16 was pipetted into six 100 mL round bottom flasks equipped with stirring bars and pH electrode. Hydrochloric acid was added dropwise with stirring to reach several pH values (pH=4, 3, 2, 1, 0) as described in the Table 17.

TABLE 17
Acid-mediated deformylation of the fermentation broth 3101

| exp | V_{FB} | V_{HCl} | V_{Total} | | pH |
|---|---|---|---|---|---|
| 1 | 50 mL | 0.0 mL | 50 | mL | 7.0 |
| 2 | 50 mL | 10.2 mL | 60.2 | mL | 4.0 |
| 3 | 50 mL | 15.6 mL | 65.6 | mL | 3.0 |
| 4 | 50 mL | 21.4 mL | 71.4 | mL | 2.0 |
| 5 | 50 mL | 35.3 mL | 85.3 | mL | 1.0 |
| 6 | 50 mL | 59.0 mL | 109.3 | mL | 0.0 |

Fermentation mixtures were stirred for 24 hours at ambient temperature (25° C.) shielded from the UV light by a wrapping the flasks in the aluminum foil. A controlled sample was prepared under the exact conditions albeit with the absence of acid (experiment 1). After 24 hours of stirring, 1 mL of each fermentation broth was diluted in duplicates with 9 mL of extraction buffer (0.1 M phosphate buffer with 1% (w/v) ascorbic acid). The suspensions were stirred on vortex, centrifuged at 4500 rpm, filtered through 0.22 μm filter and analyzed on HPLC. The HPLC results were summarized in the Table 18. According to our HPLC assay, only at lower pH levels (pH=1 and 0) deformylation provided a modest amount of folic acid. In conclusion, we have developed an acid-mediated deformylation of 10-formylfolic acid, a major product of fermentation.

TABLE 18
HPLC-based results of acid-mediated deformylation
on the fermentation broth from Example 16

| | | 5-FTHF | 10-FFA | F SUM | FA |
|---|---|---|---|---|---|
| exp | pH | mg/L | mg/L | mg/L | mg/L |
| 1 | 7.0 | 432 | 487 | 919 | 0 |
| 2 | 4.0 | 171 | 567 | 738 | 0 |
| 3 | 3.0 | 97 | 632 | 729 | 0 |
| 4 | 2.0 | 76 | 529 | 605 | 0 |
| 5 | 1.0 | 54 | 326 | 549 | 169 |
| 6 | 0.0 | 37 | 116 | 402 | 249 |

Base-Mediated Deformylation

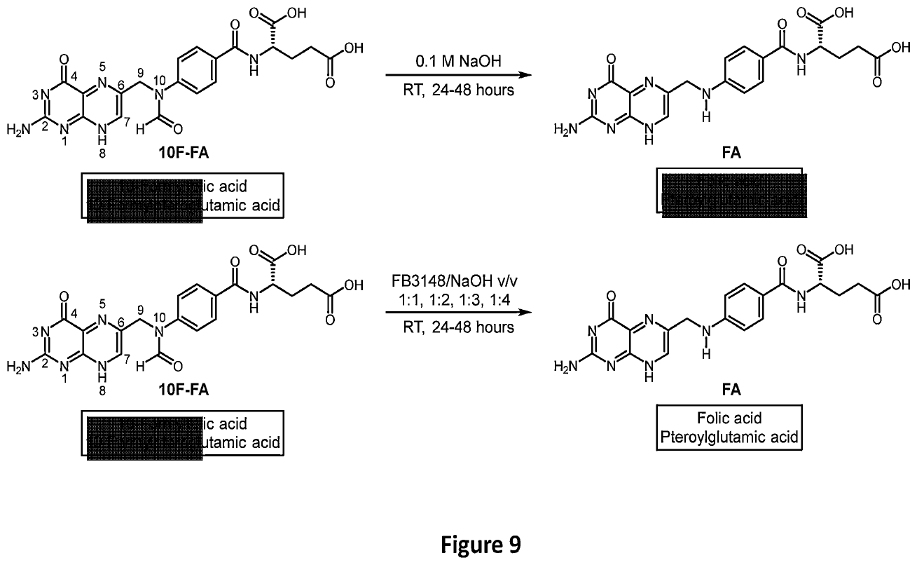

Browsing through the chemical literature, we identified a few reports describing that folic acid displays a greater stability at higher pH values. At such pH values, folic acid exhibit higher solubility which simplifies the synthetic manipulation, purification and downstream processing. Hence, in a series of N-deformylation experiments using 0.1 M NaOH, we are aiming toward clean and efficient conversion from 10-formyl folic acid to folic acid (see FIG. 9) which will simplify the isolation of target product from the fermentation broth. Initial deformylation experiments were carried out on the analytical standard of 10-formylfolic acid using 0.01 mmol scale (5 mg).

Representative Experimental Procedure:

10-formylfolic acid was weighed in the 10 mL round bottom flask equipped with a stirring bar and a rubber septum. The suspension was treated with 0.1 M sodium hydroxide (50 equiv., 0.5 mmol, 5 mL) and allowed to stir for 24-48 hours at ambient temperature protected from light. Subsequently, a solution (100 μL) was diluted with folic acid extraction buffer (900 μL), homogenized on the vortex stirrer and analyzed on HPLC. Three time-dependent aliquots were sampled analyzed on HPLC. Results of deformylation are presented in Table 19. Deformylation of 10-formylfolic acid with 0.1 M NaOH proceeded nearly quantitatively to folic acid during the first sampling after 24 hours (Table 19, entry 1). After stirring for 48 hours, the reaction proceeded to completion according to HPLC analysis. Prolonged stirring under the same conditions disclosed that newly formed folic acid did not undergo to decomposition even after 144 hours (6 days).

TABLE 19
Time scale of N-deformylation of 10-formylfolic acid to
folic acid in the presence 0.1M NaOH

| | | | | 10-FFA | | FA | |
|---|---|---|---|---|---|---|---|
| exp | reagent | time | temp | area | mg/L | area | mg/L |
| 1 | NaOH 0.1M | 24 hr | 25° C. | 16833 | 26 | 1960819 | 1167 |
| | NaOH 0.1M | 48 hr | 25° C. | 0 | 0 | 2095549 | 1247 |
| | NaOH 0.1M | 144 hr | 25° C. | 0 | 0 | 2062398 | 1228 |

Experiments were conducted in 10 mL round bottom flasks using 10-formylfolic acid (5 mg, 0.01 mmol). NaOH 0.1 M was added in excess, 50.0 equivalents, 5 mL. Mass concertation of 10-FFA at the beginning of the experiment is approximately 1000 mg/L. Progress of reaction was measured by HPLC. The HPLC samples were prepared in the extraction buffer (0.1 M phosphate buffer with 1% (w/v) ascorbic acid).

Base-Mediated N-Deformylation of 10-Formylfolic Acid in the Fermentation Broth

In previous experiments we have illustrated that deformylation of 10-formylfolic acid standard using 0.1 M NaOH provided a clean conversion to folic acid shown in FIG. 9. Herein we applied the same principle on a more complex system, a fermentation broth. HPLC analysis of fermentation broth from Example 16 before deformylation showed a substantial amount of 10-formyldihydrofolic acid (10F-DHF; 60% Area); and 10-formylfolic acid (10F-FA; 40% Area). Samples of fermentation broth from Example 16 (10 mL) were treated with different v/v ratios of 0.1 M NaOH (1:1, 1:2, 1:3 and 1:4) and stirred for 24 hours at ambient temperature (25° C.) protected from light. According to our HPLC assay, experiments with fermentation broth/NaOH v/v 1:1 and 1:2 did not lead to deformylation but to oxidative conversion of 10-formyldihydrofolic acid to of 10-formylfolic acid as displayed in Table 20 (entries 2 and 3). Subsequently, when the amount of NaOH was increased in respect to fermentation broth (1:3 and 1:4) a significant amount of folic acid was detected by HPLC as displayed in Table 20 (entries 4 and 5). Interestingly, higher amounts of NaOH somewhat hampered the oxidative conversion of 10F-DHF to 10F-FA since a substantial amount of 10F-DHF was detected by HPLC.

Representative Experimental Procedure:

Well-stirred fermentation broth from Example 16 (10 mL) was pipetted into the 50-100 mL round bottom flasks equipped with stirring bars and aluminum foil for light protection. Sodium hydroxide (0.1 M) was added dropwise and reaction was stirred vigorously for 24 hours under the ambient temperature (25° C.). After 24 hours of stirring, 1 mL of each fermentation broth was diluted in duplicates with 9 mL of extraction buffer (0.1 M phosphate buffer with 1% (w/v) ascorbic acid). The suspensions were stirred on vortex, centrifuged at 4500 rpm, filtered through 0.22 μm filter and analyzed on HPLC.

TABLE 20
Effect of addition of different amounts of NaOH on of
N-deformylation of 10-formylfolic acid in fermentation broth.

| | | | | | | | SUM |
|---|---|---|---|---|---|---|---|
| | | | | 10F-DHF | 10F-FA | FA | FOL |
| exp | sample | time | temp | mg/L | mg/L | mg/L | mg/L |
| 1 | FB3148 | 0 hr | 25° C. | 455 | 302 | 0 | 757 |
| 2 | FB3148/NaOH | 24 hr | 25° C. | 87 | 428 | 0 | 515 |
| | 0.1M (1:1 v/v) | | | | | | |
| 3 | FB3148/NaOH | 24 hr | 25° C. | 0 | 623 | 0 | 623 |
| | 0.1M (1:2 v/v) | | | | | | |
| 4 | FB3148/NaOH | 24 hr | 25° C. | 47 | 451 | 302 | 800 |
| | 0.1M (1:3 v/v) | | | | | | |
| 5 | FB3148/NaOH | 24 hr | 25° C. | 350 | 284 | 222 | 856 |
| | 0.1M (1:4 v/v) | | | | | | |

Experiments were conducted in 50-100 mL round bottom flasks using the fermentation broth from Example 16 (FB3148, 10 mL). NaOH 0.1 M was added based on the volume/volume ratio in respect to FB3148 (1:1, 1:2, 1:3 and 1:4). Progress of reaction and results were measured by HPLC. The HPLC samples were prepared in the extraction buffer (0.1 M phosphate buffer with 1% (w/v) ascorbic acid). All reactions were stirred protected from the light for 24 hours at ambient temperature (25° C.).

### Example 19: Isolation of 10-Formyl Folic Acid

After harvesting, a fermentation broth containing 50 g of folic acid was adjusted to pH=12 using 5M aqueous NaOH. The solution was centrifuged at 10000 rpm for 15 minutes at 4 ̆C. To a supernatant, 50 g of calcium hydroxide was added and suspension was stirred at room temperature for 2 hours. The resulting suspension was allowed to settle, decanted and the supernatant liquid was filtered with the aid of 100 of diatomaceous earth (Celite). The filter cake was washed with 500 mL of water and filtered. The filtrates were combined and diluted to a final volume of 10 liters. The dilute alkaline solution of clarified folic acid was adjusted to a pH 7.0 with 1N HCl, heated to 70° C. and then cooled to a room temperature. Next, the solution was filtered to remove impurities that precipitate at neutral pH. A clarified filtrate was adjusted to pH=3 using 1N HCl and cooled on ice for 4 hours. The suspension was filtered off and redissolved in 8 L of hot alkaline solution with pH=12 (adjusted with 1M NaOH). To this solution, 50 grams of activated charcoal (1 equivalent/weight of folic acid) was added and the solution was heated to 50° C. and stirred for 30 minutes. The suspension was filtered, the filter cake was washed with 3 L of alkalinized aqueous solution (pH=12 adjusted with NaOH). Filtrates were combined and pH was adjusted to 3.0 utilizing 1N HCl, added during continuous stirring. The resulting slurry was cooled on ice for 24 h or overnight. The suspension was filtered off and resuspended in 1 L of acidified aqueous solution having a pH=3 (pH was adjusted with 1N HCl). The suspension was again filtered and the resulting filter cake was then frozen and dried to obtain 43 grams of folic acid, which contained 10% of moisture and assayed 90.1. % folic acid on an anhydrous basis.

### Example 20: Isolation of Folic Acid

After harvesting, a fermentation broth containing 30 g of folic acid was adjusted to pH=10 using 1M aqueous NaOH. The solution was centrifuged at 10000 rpm for 15 minutes at 4 ̆C. The resulting supernatant was adjusted to a pH 4.0 with 1N HCl, heated to 70° C. and then cooled to a room temperature. Next, the solution was filtered with the aid of 100 g of Celite. Filter cake was resuspended in 5 L of alkaline solution with pH=10 (adjusted with 1M NaOH). To this solution, 50 grams of activated charcoal (1 equivalent/weight of folic acid) was added and the solution was heated to 50° C. and stirred for 30 minutes. The suspension was filtered, the filter cake was washed with 2 L of alkalinized aqueous solution (pH=12 adjusted with NaOH). Filtrates were combined and pH was adjusted to 3.0 utilizing 1N HCl, added during continuous stirring. The resulting precipitate was cooled on ice for 16-24 h or then filtered off and resuspended in 1 L of acidified aqueous solution having a pH=3 (pH was adjusted with 1N HCl). The suspension was again filtered and the resulting precipitate cake was dried to obtain 21 grams of 10-formyl folic acid, which was assayed 92%.

### Example 21. Assembly of 5-Methylfolate Operon (MTHF-OP)

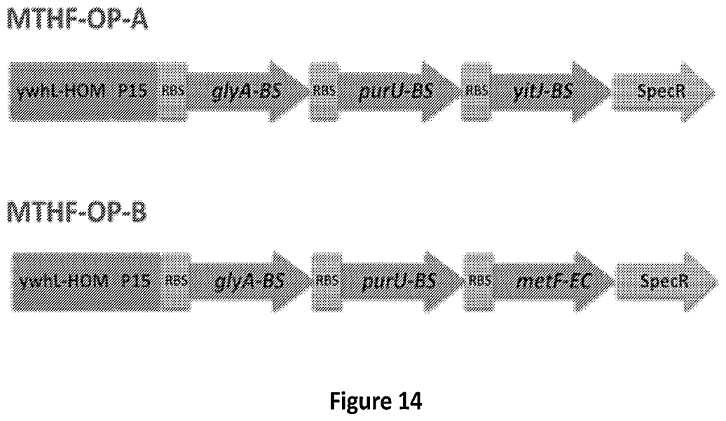

The synthesis of folate biosynthetic genes (glyA, purU yitJ and metF) was carried out as separate synthetic DNA fragments (SEQ ID NO: 91, 92, 93 and 94) with gene nucleotide sequences codon-optimized for B. subtilis optimal expression. The fragments were assembled into artificial operon by repetitive steps of restriction and ligation (Example 3). A combination of NdeI and AseI restriction sites is used in order to assure compatible restriction ends for successful ligation. After each step of ligation, the combined fragments were used as a new template for next PCR amplification. Operons were assembled stepwise in order to combine different biosynthetic genes with homologies for ywhL locus (SEQ ID NO: 95) and spectinomycin selectable marker (SEQ ID NO: 96). Integration locus ywhL (uncharacterized protein) was selected as a new chromosome integration site. Folate biosynthetic genes methyl-folate operons (MTHF-OP) were design as a combination of genes involved in the final steps of 5-methyltetrahydrofolate biosynthesis and are under control of a strong constitutive P15 promoter (FIG. 14). In the MTHF-OP-A operon (SEQ ID NO: 97) genes (glyA, purU and yitJ) were selected from native host organism B. subtilis and additional codon-optimized for B. subtilis optimal gene expression. Additionally, homologue gene (metF) from E. coli were used for construction of alternative to yitJ gene in operon MTHF-OP-B (SEQ ID NO: 98).

### Example 22: Mutagenesis of B. subtilis

Ethyl methanesulfonate (EMS) mutagenesis was performed on B. subtilis strain FL825. The culture was grown in liquid LB medium with appropriate antibiotics to the exponential phase. Then, the culture was centrifuged at 3000 RPM for 3-5 min, and the supernatant was removed. The cell pellet was washed twice in sterile 0.9-% NaCl, and the supernatant was removed by centrifugation. The pellet was resuspended in 0.9-% NaCl. One hundred of cell suspension was diluted in 900 μL of 3% Ethyl methanesulfonate (EMS). The cells were exposed to EMS for corresponding time with constant agitation/mixing. After the incubation, 1 volume of 10-15% Na-thiosulfate was added per 1 volume of cell suspension to stop the mutagenesis reaction. The liquid was removed by centrifugation. The cell pellet was washed in sterile 0.9-% NaCl twice, and the supernatant was removed by centrifugation. Serial dilutions of the mutagenized cells were plated on MB plates with appropriate antibiotic and incubated for approximately 48 h at 37° C. Single colonies were further tested for 5-methyltetrahydrofolate production (Example 25).

### Example 23: Determination of the Expression Level of metE Gene Using qPCR

B. subtilis strain FL2771 was grown in liquid LB medium to the exponential phase. The culture was mixed with 2 volumes of the RNAprotect Bacteria Reagent (QIAGEN), centrifuged for 10 min at 4500 rpm and frozen at −80° C. or processed immediately. The cell pellet was resuspended in 200 μL of TE buffer containing 1 mg/mL lysozyme for 15 min to remove the cell wall. RNA was isolated by using RNeasy mini kit (QIAGEN) according to the manufacturer's protocol. The obtained RNA was checked for concentration and quality spectrophotometrically. The isolated RNA was treated with DNase (Ambion kit) and reverse-transcribed to cDNA by using RevertAid H Minus First Strand cDNA Synthesis Kit (Thermo Scientific). The obtained cDNA was diluted and the final yield of cDNA was app 2.5 ng/μL.

The obtained cDNA was analysed by real time quantitative PCR (qPCR) technique (StepOne Real-Time PCR System, Applied Biosystems) with SYBR Green I (Thermo Scientific) detection. The expression of the metE gene in the 5-Me-THF-producing strain(s) was quantified by qPCR.

As an internal control gene, 16S rRNA gene from B. subtilis was used as a reference for normalization of the quantitative qPCR expression data. B. subtilis VBB38 was used as the control strain. The expression of metE gene was determined using specific pair of primers Q_metE_F (SEQ ID NO:73) and Q_metE_R (SEQ ID NO:74) and for 16S gene selected as the internal control gene, primer pair SEQ ID NO:69 and SEQ ID NO:70 were used. The qPCR analysis was run on StepOne™ Real-Time PCR System and quantification was performed by using the 2-ΔΔCT method.

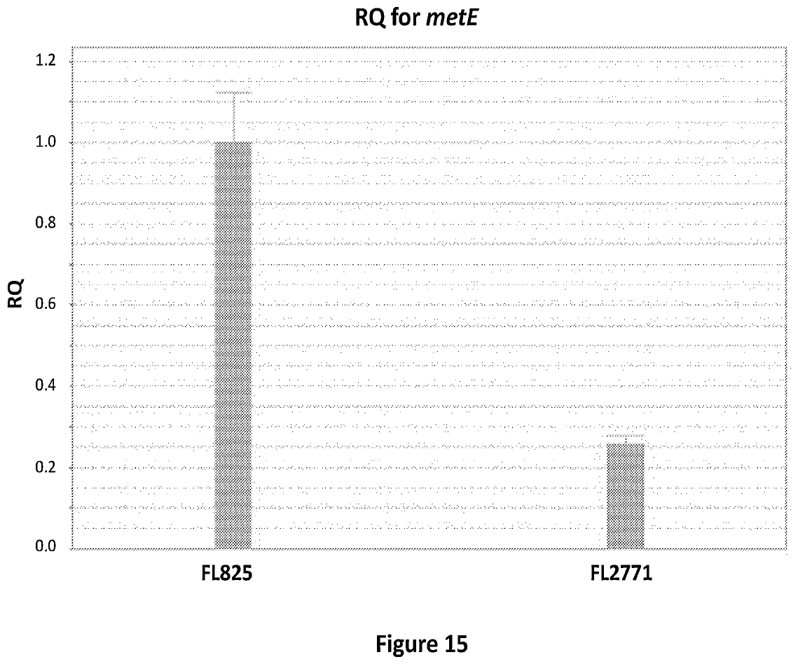

Strain FL2771 had downregulated expression of metE gene compared to starting parent strain FL825 for more than 70% (FIG. 15).

### Example 24: Generation of 5-Methyl Tetrahydrofolate-Producing Strains

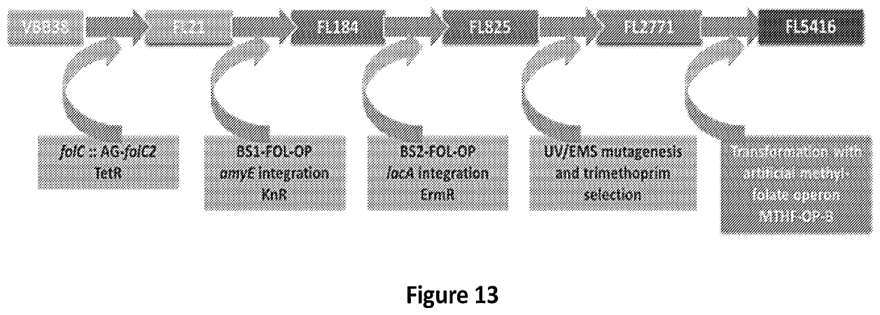

5-Methyl tetrahydrofolate-producing strains were developed from starting strain VBB38 (see FIG. 13). Two important genetic modification were performed in order to improve total folate production capacity of the engineered strains. Initially native folC gene in B. subtilis was disrupted in order to abolish the synthesis of γ-glutamate tail on folates and replaced with folC homolog encoding only for the dihydrofolate synthetase (DHFS) activity, resulting in the addition of only one essential glutamate moiety without the γ-glutamate tail. Therefore, strain FL21 was developed with phenotype capable of enhanced excretion of folates from the cells to the fermentation medium in order to assure high metabolic flow through the folate biosynthetic pathway. In the following step of strain development overexpression of the folate biosynthetic genes (such as, folE/mtrA, folB, folK, folP/sul, folA/dfrA) were introduced in order to upregulate “de novo” biosynthetic pathway (from GTP precursor to THF). Artificial folate operons (BS-FOL-OP1 and BS-FOL-OP2—see Example 3) were constructed bearing selected folate biosynthetic genes and integrated into B. subtilis at multiple genome locations (amyE and lacA locus) in order to provide high level of gene expression. Upregulation of folate biosynthetic genes in newly engineered strain FL184 (BS-FOL-OP1) and strain FL825 (BS-FOL-OP1+ BS-FOL-OP2) has significantly improved production capacity of total folates compared to the starting strain.

Further engineered strains of B. subtilis for overproduction of 5-methyl tetrahydrofolate were generated by downregulation/deletion of native metE gene and overexpression of genes involved in interconversion of folate forms (from THF towards 5-methyltetrahydrofolate) (such as, glyA, purU, yitJ and metF). Downregulation of native metE gene was achieved by random mutagenesis of parent strain FL825 and mutant strain selection based on their improved 5-methyltetrahydrofolate production. New strain FL2771 was generated, reaching significantly higher titer of 5-methyltetrahydrofolate (more than 220 mg/L) compared with the wild-type strain (0.35 mg/L). The strain FL2771 was further selected for whole genome sequencing. Bioinformatic analyses of FL2771 genome and comparison to whole genome sequence data of ancestor starting strain B. subtilis VKPM B2116 was able to rationally connect the several observed SNP variances/mutations, introduced by random mutagenesis during strain development, with folate metabolic cycle. One mutation is located directly upstream of the metE gene (SEQ ID NO: 75 and SEQ ID NO: 76), this mutation is located in the regulatory region of the gene coding for methionine synthase involved in consumption of 5-methyltetrahydrofolate.

In the last step of developing 5-methyltetrahydrofolate producing strain, we have performed overexpression of genes (glyA, purU, yitJ and metF) crucial for interconversion of folate forms. Overexpression of selected genes was design as a new methyl folate biosynthetic operon (MTHF-OP) constructed from genes involved in final steps of 5-methyltetrahydrofolate biosynthesis (glyA and yitJ/metF) and gene purU (formyltetrahydrofolate deformylase) involved in conversion of 10F-THF to the THF in order to improve ratio between 10F-folates and 5-methyltetrahydrofolate. New genetically engineered strain FL5416 has demonstrated to produce the highest 5-methyltetrahydrofolate yield of more than 300 mg/L whereas 5-methyltetrahydrofolate form represented 75% of total folates measured.

### Example 25: Determination of Ratio of Different Forms of Folates in Genetically Engineered Strains of B. subtilis

Strains were patched on MB plates with appropriate antibiotics and incubated at 37° C. for 2 days. For shake-flasks experiments, the grown strains were transferred to 5 ml of MC (seed) medium in Falcon 50 mL conical centrifuge tubes (1 plug/5 ml) and cultivated on a rotary shaker at 220 RPM and 37° C. for 16-18 h. A 10-% inoculum of the seed culture was used to inoculate 5 mL of the production medium (MD+pABA500). The strains were cultivated on a rotary shaker at 220 RPM and 37° C. for 24 h in the dark. After the fermentation, the samples of the fermentation broth (200 μl) was carefully collected to obtain a homogeneous sample and diluted 10 times in the ice-cold extraction buffer (0.1 M phosphate buffer with 1% (w/v) ascorbic acid). The samples were centrifuged at 14,000 rpm and 4° C. for 10 min and filter-sterilized (0.22 um pore size). For the quantification of different folate species HPLC method was used as described in Example 13. Results of different B. subtilis strain are shown in Table 21 and representative HPLC chromatogram of fermentation broth sample is shown in FIG. 6.

TABLE 21
Folate production of different Bacillus subtills strains
in experiments at shaker scale (5 ml) after 24 hours.

| | | 5M-THF | FA | 10F-DHF | 10F-FA |
|---|---|---|---|---|---|
| Strain | Strain description | (mg/L) | (mg/L) | (mg/L) | (mg/L) |
| wt 168 | B. subtillis wild type | 0.16 | 0.15 | 0.01 | 0.86 |
| VBB38 | B. subtilis VKPM | 0.35 | 0.01 | 0.02 | 0.09 |
| | B2116 | | | | |
| FL21 | VBB38 folC::tetR | 18.20 | 0.01 | 0.03 | 2.64 |
| | P-veg/folC2-AG | | | | |
| FL23 | VBB38 folC::tetR | 1.11 | 0.01 | 0.01 | 0.03 |
| | P-veg/folC2-LR | | | | |
| FL184 | FL21 amyE::kanR | 48.5 | 0.03 | 8.1 | 186.6 |
| | P-veg/FOL-OP-BS1 | | | | |
| FL825 | FL184 lacA::EryR | 35.1 | 0.07 | 14.6 | 328.9 |
| | P-15/FOL-OP-BS2 | | | | |
| FL2771 | FL825 (EMS | 227.8 | 0.02 | 6.2 | 99.2 |
| | mutagenesis) metE* | | | | |
| FL5416 | FL2771 | 306.5 | 0.02 | 2.7 | 24.0 |
| | ywhL::SpecR P-15/ | | | | |
| | MTHF-OP-B | | | | |

Strain FL5416 with heterologous folC-AG and overexpressed folate biosynthetic genes (folate operon FOL-OP-BS1, FOL-OP-BS2 and MTHF-OP-B) showed 191462% increased 5-methylfolate production compared to the wild type strain Bacillus subtilis 168.

### Comparative Example 1

Total folate production was determined for B. subtilis wild type strain “168”, our starting non-GMO strain VBB38 (strain VKPM B2116=B. subtilis VNII Genetika 304) and its transformants in which native folC gene was replaced in one step by a heterologous folC2 (FOL3) gene from either A. gossypii (B. subtilis strain FL21) or L. reuteri (B. subtilis strain FL23). Strains were tested at the shaker scale (5 ml production medium MD) and total folates were determent by using standard microbiological assay for folate detection.

The result was shown that knockout mutants of deletion of B. subtilis native folC gene alone without simultaneous heterologous folC2 gene expression were not able to grow in standard cultivation conditions (T=37 C, aerobically in nutrient rich LB medium).

### LIST OF REFERENCES CITED IN THE DESCRIPTION

Hjortmo S, Patring J, Andlid T. 2008 Growth rate and medium composition strongly affect folate content in Saccharomyces cerevisiae. Int J Food Microbiol. 123(1-2):93-100.
McGuire J J and Bertino J R. 1981. Enzymatic synthesis and function of folylpolyglutamates. Mol Cell Biochem 38 Spec No (Pt 1):19-48.
Reed, L S, Archer M C. 1980. Oxidation of tetrahydrofolic acid by air. J Agric Food Chem. 28(4):801-805.
Rossi, M., Raimondi, S., Costantino, L., Amaretti, A., 2016. Folate: Relevance of Chemical and Microbial Production. Industrial Biotechnology of Vitamins, Biopigments, and Antioxidants. Wiley-VCH Verlag GmbH & Co. KGaA, Weinheim, Germany, pp. 103-128.
Scaglione and Panzavolta. 2014. Folate, folic acid and 5-methyltetrahydrofolate are not the same thing. Xenobiotica. 44(5):480-488.
Serrano-Amatriain C, Ledesma-Amaro R, Lopez-Nicolas R, Ros G, Jimenez A, Revuelta J L. 2016. Folic acid production by engineered Ashbya gossypii. Metab Eng. 38:473-482.
Sybesma W, Starrenburg M, Kleerebezem M, Mierau I, de Vos W M, Hugenholtz J. 2003a. Increased production of folate by metabolic engineering of Lactococcus lactis. Appl Environ Microbiol. 69(6):3069-3076.
Sybesma, W., Starrenburg, M., Tijsseling, L., Hoefnagel, M. H. N., Hugenholtz, J., 2003b. Effects of cultivation conditions on folate production by lactic acid bacteria. Applied and Environmental Microbiology. 69(8):4542-4548.
Sybesma W, Van Den Born E, Starrenburg M, Mierau I, Kleerebezem M, De Vos W M, Hugenholtz J. 2003c. Controlled modulation of folate polyglutamyl tail length by metabolic engineering of Lactococcus lactis. Appl Environ Microbiol. 69(12):7101-7107.
Walkey C J, Kitts D D, Liu Y, van Vuuren H J J. 2015. Bioengineering yeast to enhance folate levels in wine. Process Biochem 50(2):205-210.
Qui Z and Goodman M F: The Escherichia coli polB locus is identical to dinA, the structural gene for DNA polymerase II. Characterization of Pol II purified from a polB mutant. J Biol Chem. 1997, 272(13): 8611-8617.
Kwon D H, Peña JA, Osato M S, Fox J G, Graham D Y, Versalovic J: Frameshift mutations in rdxA and metronidazole resistance in North American Helicobacter pylori isolates. J Antimicrob Chemother 2000, 46(5): 793-796
Datsenko K A, Wanner B L: One-step inactivation of chromosomal genes in Escherichia coli K-12 using PCR products. Proc Natl Acad Sci USA 2000, 97:6640-6645.

1. A genetically engineered microorganism, comprising:
a modification producing an increased expression level of at least one enzyme involved in the biosynthesis of a 5-methylfolate compared to an otherwise identical microorganism that does not comprise said modification; or
a modification producing a decreased expression or activity of an endogenous polypeptide having 5-methyltetrahydropteroyltriglutamate-homocysteine S-methyltransferase activity compared to an otherwise identical microorganism that does not comprise said modification.

2-15. (canceled)

16. The genetically engineered microorganism according to claim 1, wherein said microorganism comprises a modification producing an increased expression level of at least one enzyme involved in the biosynthesis of a 5-methylfolate compared to an otherwise identical microorganism that does not carry said modification.

17. The genetically engineered microorganism according to claim 1, wherein the enzyme involved in the biosynthesis of a 5-methylfolate is selected from the group consisting of: a polypeptide having GTP cyclohydrolase activity, a polypeptide having 7,8-dihydroneopterin aldolase activity, a polypeptide having 2-amino-4-hydroxy-6-hydroxymethyldihydropteridine pyrophosphokinase activity, a polypeptide having dihydropteroate synthase activity, a polypeptide having dihydrofolate reductase activity, a polypeptide having serine hydroxymethyltransferase activity, a polypeptide having formyltetrahydrofolate deformylase activity, and a polypeptide having 5,10-methylenetetrahydrofolate reductase activity.

18. The genetically engineered microorganism according to claim 1, wherein said microorganism comprises a modification producing an increased expression level of a polypeptide having 5,10-methylenetetrahydrofolate reductase activity compared to an otherwise identical microorganism that does not carry said modification.

19. The genetically engineered microorganism according to claim 1, wherein the at least one enzyme involved in the biosynthesis of a 5-methylfolate is heterologous to the genetically engineered microorganism.

20. The genetically engineered microorganism according to claim 1, wherein said microorganism comprises a modification producing a decreased expression or activity of an endogenous polypeptide having 5-methyltetrahydropteroyltriglutamate-homocysteine S-methyltransferase activity compared to an otherwise identical microorganism that does not carry said modification.

21. The genetically engineered microorganism according to claim 1, wherein said microorganism comprises a modification producing:
a) a decreased expression or activity of an endogenous polypeptide having both dihydrofolate synthase activity and folylpolyglutamate synthetase activity compared to an otherwise identical microorganism that does not comprise said modification, and
b) expression of a heterologous polypeptide having only dihydrofolate synthase activity.

22. The genetically engineered microorganism according to claim 1, wherein said microorganism is a bacterium.

23. The genetically engineered microorganism according to claim 22, wherein said bacterium is of the species Bacillus subtilis.

24. A method for preparing a folate, precursor or intermediate thereof, comprising:
i) cultivating the genetically engineered microorganism according to claim 1 in a culture medium under suitable culture conditions to obtain a fermentation product containing said folate, precursor or intermediate thereof; and
ii) optionally, separating or purifying said folate, precursor or intermediate thereof.

25. The method according to claim 24, wherein the folate is a compound of Formula I: 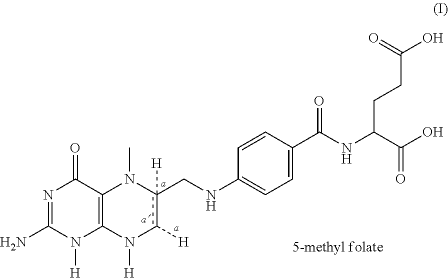
optionally in the form of one of the stereoisomers, a racemate or in form of a mixture of at least two of the stereoisomers in any mixing ratio.

26. The method according to claim 24, wherein the folate is a compound of Formula II: 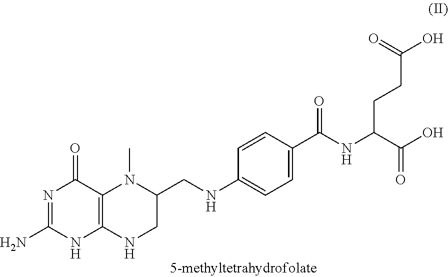 [SMILES: CN1c2c([nH]c(N)nc2=O)NCC1CNc1ccc(C(=O)NC(CCC(=O)O)C(=O)O)cc1]
optionally in form of one of the stereoisomers, a racemate or a mixture of at least two of the stereoisomers in any mixing ratio.

27. The method according to claim 24, wherein the folate is a compound of Formula IIa:

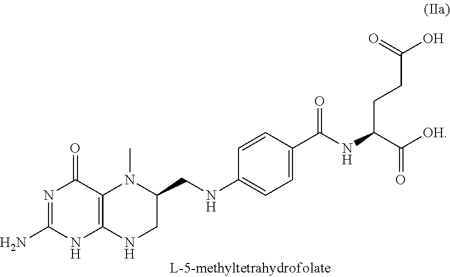 [SMILES: CN1c2c([nH]c(N)nc2=O)NC[C@@H]1CNc1ccc(C(=O)N[C@@H](CCC(=O)O)C(=O)O)cc1]

28. A method of preparing a genetically engineered microorganism, comprising:
(a) increasing the expression level of at least one enzyme involved in the biosynthesis of a 5-methylfolate compared to an otherwise identical microorganism; or
(b) decreasing the expression or activity of an endogenous polypeptide having 5-methyltetrahydropteroyltriglutamate-homocysteine S-methyltransferase activity compared to an otherwise identical microorganism that does not comprise said modification.

29. The method according to claim 28, further comprising:
decreasing the expression or activity of an endogenous polypeptide having both dihydrofolate synthase activity and folylpolyglutamate synthetase activity compared to an otherwise identical microorganism that does not comprise said modification; and
expressing a heterologous polypeptide having dihydrofolate synthase activity.


SEQUENCE LISTING (101 sequences)

SEQ ID NO: 1 - DNA, 573 bp, Bacillus subtilis
atgaaagaagttaataaagagcaaatcgaacaagctgttcgtcaaattttagaagcgatcggagaagacccgaatagagaagggcttcttgatactccgaaaagagtcgcaaagatgtatgccgaagtattctccggcttgaatgaagatccaaaagaacatttccagactatcttcggtgaaaaccatgaggagcttgttcttgtaaaagatatagcgtttcattctatgtgtgagcatcaccttgttcccttttatggaaaagcacatgttgcatatatcccgcgaggcggaaaggtcacaggactcagcaaactggcacgtgccgttgaagccgttgcaaagcgcccgcagcttcaggaacgcatcacttctacaattgcagaaagcatcgtagaaacgcttgatccgcatggcgtaatggtagtggttgaagcggaacacatgtgcatgacgatgcgcggtgtaagaaaaccgggtgcgaaaactgtgacttcagcagtcagaggcgtttttaaagatgatgccgctgcccgtgcagaagtattggaacatattaaacgccaggactaa

SEQ ID NO: 2 - DNA, 363 bp, Bacillus subtilis
atggataaagtttatgtagaaggtatggagttttacggatatcacggtgtgttcacagaagaaaacaaacttggccagcggtttaaagtcgatttaaccgctgagctggatttaagcaaagctggacagacagacgaccttgagcaaacgatcaactatgctgagctctatcacgtatgtaaagatatcgtggaaggggagcctgtgaaattggtggaaacgctggcggaacgtattgctggcactgttctcggaaaatttcagcctgttcagcaatgtacggtgaaagtgattaagccagacccgccaattcccggacactataaatcagtagcaattgaaattacgagaaaaaagtcatga

SEQ ID NO: 3 - DNA, 504 bp, Bacillus subtilis
atgaacaacatagcttatattgcacttggatctaatattggagatagagaaacgtatttaaggcaagcagtggctttactgcatcagcatgctgcggtgacagtcactaaagtgtcgtctatttacgaaactgacccggtcggatacgaagatcaagctcaatttttgaatatggctgttgaaatcaagacatcattgaacccttttgaactccttgaactgacgcagcagatagaaaatgaattaggcagaacaagggaagtaagatgggggccgcggacggcagaccttgacattttgttatttaatcgtgaaaatattgaaacagagcaactaattgttccgcatccgagaatgtatgagcgtttgtttgtccttgcgccgcttgcggaaatttgccagcaggttgaaaaagaggctacaagcgccgaaacagaccaagaaggtgtaagagtatggaagcagaaatctggggtagacgaattcgtgcattcagaaagctga

SEQ ID NO: 4 - DNA, 858 bp, Bacillus subtilis
atggcgcagcacacaatagatcaaacacaagtaatccacactaagcccagcgctttatcatataaagagaagacgctggtgatgggaattttaaacgtaacgcctgactctttctcggacggcggaaaatatgacagcttggacaaggcgctgctgcacgcgaaagagatgatcgatgatggtgcccatatcattgatattggaggggaatcgacaaggcctggcgctgagtgcgtatctgaggatgaggagatgtccagagtcattccggtgattgagcggattacgaaagagcttggtgttcctatttctgtagacacgtacaaggcttctgtcgcagatgaagcagtgaaagccggtgcatccattatcaatgatatttggggagccaaacatgatccgaagatggcttccgttgcagctgaacataatgttccaattgtactcatgcataaccgccctgaaagaaactacaatgacttattgccggatatgctgtcggacttaatggagagtgtaaaaattgctgttgaggccggagtagacgagaagaacattattcttgatcctggtatcggtttcgcgaaaacctatcacgataacttggcagtgatgaacaaactagagattttcagcggattgggatatccggttcttctggcaacctcccgaaaaagattcatcggacgtgttctggatcttccgcctgaggagcgggctgagggcacaggcgcgactgtgtgtctcggcattcaaaaaggctgtgacattgtcagggtccatgatgtaaagcaaattgccagaatggcgaaaatgatggacgcgatgctgaataagggaggggtgcaccatggataa

SEQ ID NO: 5 - DNA, 1293 bp, Bacillus subtilis
ttgtttactgcatatcaagatgcgcgcagctggattcacgggaggctgaaattcggggtgaagcccggacttggtcgaatgaaacagctgatggcgcggttaggacatcctgaaaaaaaaatccgagcgttccacgtcgcaggaacaaacggtaaaggatcaactgtcgcttttatccgttctatgctgcaggaagccggatatacggttggaacatttacatcgccttatattattacgtttaatgaacggatcagcgtaaacgggataccgatttcagatgaggaatggacagcactcgtcaatcaaatgaaaccgcacgttgaagcccttgatcaaacggaatacggacagccgacagaatttgaaattatgacagcttgtgcatttttatattttgcagagtttcataaggtcgattttgttatttttgaaacaggtctgggcggtagattcgattctactaatgtggttgaaccgctcttaactgtgattacaagcatcggacacgatcatatgaacattttgggaaacaccattgaagaaattgcaggagaaaaggccggcattattaaagaaggtattccaatcgttacagcagttacccagccggaagctttacaggtcatccgccatgaagccgaacggcatgctgcgccgttccagtcattgcatgatgcatgtgttattttcaatgaagaggctctgcctgcaggagaacagttttcattcaaaacggaagagaaatgctatgaagatattcggacatctctgattggcacccatcaaagacaaaatgctgctttgtccattttggctgcggagtggctcaataaggagaatatcgcccatatcagtgacgaagcgttaaggagcgggcttgtgaaggctgcttggccgggacggttagagcttgttcaggaacatcctccggtatatttagacggtgcgcataacgaagaaggtgttgaaaagctggcggaaacgatgaagcagcgtttcgccaattcacgcatttctgtggtgtttagcgcgttaaaggacaaaccgtatcagaacatgattaaaagactggaaaccattgctcatgcgattcattttgcttcttttgatttcccgcgtgcttcccttgcaaaagatctctatgatgcaagtgaaatcagcaataaaagctggagtgaagatccagacgatgtaatcaaattcatagaaagtaaaaagggctcaaatgaaattgtgctgattaccggatctctttactttatttctgacattcgaaaaaggttgaaataa

SEQ ID NO: 6 - DNA, 507 bp, Bacillus subtilis
atgatttcattcatttttgcgatggatgccaacaggcttatcggcaaagacaatgatttgccgtggcatttgcccaatgatcttgcatactttaagaaaataacatcgggccattcaatcattatgggccggaaaacatttgaatcgatcggacgtccgcttccaaatcggaaaaatattgtcgttacctcagcgccggattcagaatttcagggatgcacggttgtcagttcattaaaggatgtactggacatttgttcaggccctgaagaatgctttgtgatcggaggggctcagctctatacggacctgttcccttatgcggacagactgtatatgacgaaaattcatcacgagtttgagggtgaccgtcactttcctgaatttgatgaatccaattggaagctggtttcttctgagcaggggaccaaagacgaaaaaaacccgtatgattacgaatttctaatgtatgaaaaaaagaattcttctaaagcgggaggattttaa

SEQ ID NO: 7 - protein, 190 aa, Bacillus subtilis
MKEVNKEQIEQAVRQILEAIGEDPNREGLLDTPKRVAKMYAEVFSGLNEDPKEHFQTIFGENHEELVLVKDIAFHSMCEHHLVPFYGKAHVAYIPRGGKVTGLSKLARAVEAVAKRPQLQERITSTIAESIVETLDPHGVMVVVEAEHMCMTMRGVRKPGAKTVTSAVRGVFKDDAAARAEVLEHIKRQD

SEQ ID NO: 8 - protein, 120 aa, Bacillus subtilis
MDKVYVEGMEFYGYHGVFTEENKLGQRFKVDLTAELDLSKAGQTDDLEQTINYAELYHVCKDIVEGEPVKLVETLAERIAGTVLGKFQPVQQCTVKVIKPDPPIPGHYKSVAIEITRKKS

SEQ ID NO: 9 - protein, 167 aa, Bacillus subtilis
MNNIAYIALGSNIGDRETYLRQAVALLHQHAAVTVTKVSSIYETDPVGYEDQAQFLNMAVEIKTSLNPFELLELTQQIENELGRTREVRWGPRTADLDILLFNRENIETEQLIVPHPRMYERLFVLAPLAEICQQVEKEATSAETDQEGVRVWKQKSGVDEFVHSES

SEQ ID NO: 10 - protein, 285 aa, Bacillus subtilis
MAQHTIDQTQVIHTKPSALSYKEKTLVMGILNVTPDSFSDGGKYDSLDKALLHAKEMIDDGAHIIDIGGESTRPGAECVSEDEEMSRVIPVIERITKELGVPISVDTYKASVADEAVKAGASIINDIWGAKHDPKMASVAAEHNVPIVLMHNRPERNYNDLLPDMLSDLMESVKIAVEAGVDEKNIILDPGIGFAKTYHDNLAVMNKLEIFSGLGYPVLLATSRKRFIGRVLDLPPEERAEGTGATVCLGIQKGCDIVRVHDVKQIARMAKMMDAMLNKGGVHHG

SEQ ID NO: 11 - protein, 430 aa, Bacillus subtilis
MFTAYQDARSWIHGRLKFGVKPGLGRMKQLMARLGHPEKKIRAFHVAGTNGKGSTVAFIRSMLQEAGYTVGTFTSPYIITFNERISVNGIPISDEEWTALVNQMKPHVEALDQTEYGQPTEFEIMTACAFLYFAEFHKVDFVIFETGLGGRFDSTNVVEPLLTVITSIGHDHMNILGNTIEEIAGEKAGIIKEGIPIVTAVTQPEALQVIRHEAERHAAPFQSLHDACVIFNEEALPAGEQFSFKTEEKCYEDIRTSLIGTHQRQNAALSILAAEWLNKENIAHISDEALRSGLVKAAWPGRLELVQEHPPVYLDGAHNEEGVEKLAETMKQRFANSRISVVFSALKDKPYQNMIKRLETIAHAIHFASFDFPRASLAKDLYDASEISNKSWSEDPDDVIKFIESKKGSNEIVLITGSLYFISDIRKRLK

SEQ ID NO: 12 - protein, 168 aa, Bacillus subtilis
MISFIFAMDANRLIGKDNDLPWHLPNDLAYFKKITSGHSIIMGRKTFESIGRPLPNRKNIVVTSAPDSEFQGCTVVSSLKDVLDICSGPEECFVIGGAQLYTDLFPYADRLYMTKIHHEFEGDRHFPEFDESNWKLVSSEQGTKDEKNPYDYEFLMYEKKNSSKAGGF

SEQ ID NO: 13 - DNA, 683 bp, Artificial Sequence
  Optimized folE sequence for Bacillus subtilis codon usage
cgcagcatacgcagcgaaatcagcatcaccggagaatcccaacgaagccaactagtatgaaagaagtcaataaagaacaaattgaacaggcagtgagacagattcttgaagcaattggagaagatccgaacagagagggcttacttgatacaccgaaaagagttgctaaaatgtatgcggaagtcttttcaggcttaaatgaagatccgaaagagcattttcagacaattttcggagaaaaccatgaagagctggtccttgtgaaagatattgcgtttcactcaatgtgcgaacatcacctggtgccgttttacggcaaggcacacgttgcgtatattcctagaggcggaaaagttacaggcttgtcaaaattagcacgcgcagttgaagctgttgcaaaaagaccgcaattacaggaacgcattacatctacaattgcggaatcaattgtcgagacattagaccctcatggcgttatggttgtcgttgaagctgaacacatgtgcatgacaatgcgcggcgtcagaaaacctggcgcaaaaacagtcacatcagcagtcagaggcgtgtttaaagatgatgcggcagctcgtgcggaagtcctggaacatattaaacgccaggactgaaaaagaggggagggaaacattaatgacgacctggctaacgagtctcgccgatct

SEQ ID NO: 14 - DNA, 454 bp, Artificial Sequence
  Optimized  folB sequence for Bacillus subtilis codon usage
ttctttttgcgccaggtagccatagctggtcatatgatggataaagtttatgtggaaggaatggaattttatggctatcatggcgtcttcacagaagagaacaaattgggacaacgcttcaaagtagatctgacagcagaactggatttatcaaaagcaggacaaacagacgaccttgaacagacaattaactatgcagagctttaccatgtctgtaaagacattgtcgaaggcgagccggtcaaattggtagagacccttgctgagcggatagctggcacagttttaggtaaatttcagccggttcaacaatgtacggtgaaagttattaaaccagatccgccgattcctggccactataaatcagtagcaattgaaattacgagaaaaaagtcataaattaattctagagtcgatccccgggttcgccagcaatgactaccggcagcccgcc

SEQ ID NO: 15 - DNA, 595 bp, Artificial Sequence
  Optimized folK sequence for Bacillus subtilis codon usage
ggcggggcttcttttatcgaatccagcgtgacatatgatgaacaacattgcgtacattgcgcttggctctaatattggagatagagaaacgtatctgcgccaggccgttgcgttactgcatcaacatgctgcggtcacagttacaaaagtcagctcaatttatgaaacagatccggtcggctatgaagaccaagcccagtttttaaatatggcggttgaaattaaaacaagcctgaatccgtttgaacttctggaactgacacagcaaatcgaaaacgaactgggccgcacacgcgaagttagatggggcccgagaacagcggatttagacattctgctgtttaacagagaaaacattgaaacagagcagttaattgtcccgcatcctcgcatgtatgaacgcctgtttgttcttgcgccgcttgcggaaatttgccagcaggtcgagaaagaagcgacaagcgcggaaacggatcaagaaggagttagagtttggaaacaaaaatcaggcgttgacgaatttgtacatagcgaaagctgaaaaagaggggagggaaacattaatgaccgaccctcatggaaacccttcctggcg

SEQ ID NO: 16 - DNA, 948 bp, Artificial Sequence
  Optimized sul sequence for Bacillus subtilis codon usage
gaccgaccctcatggaaacccttcctggcgcatatgatggcgcagcacacaatagatcaaacacaagtcattcatacgaaaccgagcgcgctttcatataaagaaaaaacactggtcatgggcattcttaacgttacacctgattcttttagcgatggtggaaaatatgacagcttggacaaggcgcttctgcatgccaaagaaatgatcgacgacggcgcgcacattattgacataggaggcgagagcacaagaccgggagctgaatgcgtcagcgaagacgaagaaatgtctcgggtcattccggtcattgaacgcatcacaaaggaactcggcgtcccgatttcagtggatacatataaagcatctgtggcagacgaagcagtcaaagcgggcgcatctattatcaatgacatttggggagcgaaacatgatccgaagatggcaagcgtcgcagcggaacataacgttccaattgtcctgatgcacaatcggccagaacggaattataacgaccttcttccggatatgctgagcgaccttatggaatcagtcaaaattgcggttgaggcgggcgtggatgagaaaaatattattttagatccgggcatcggcttcgcgaagacataccatgataatcttgcagtgatgaataagttagagatcttcagcggacttggctatcctgtcctgctggctacatctcgtaaaagatttatcggaagagttcttgatttaccgcctgaagagagagcagagggcacaggagcgacagtctgcttgggcattcagaaaggatgcgacatagtgcgtgttcatgatgtcaagcaaattgccagaatggcgaaaatgatggacgcgatgctgaataagggaggggtgcaccatggatgaaaaagaggggagggaaacattaatttctttttgcgccaggtagccatagctggt

SEQ ID NO: 17 - DNA, 598 bp, Artificial Sequence
  Optimized dfrA sequence for Bacillus subtilis codon usage
gacgacctggctaacgagtctcgccgatctcatatgatgatttcatttattttcgcaatggacgcgaatagactgataggcaaagacaatgatctgccgtggcatttaccgaatgacctggcttattttaaaaaaattacaagcggccatagcatcattatgggacgtaaaacatttgagtcaattggcagacctcttccgaacagaaaaaacattgttgtcacatctgcgccggattcagaatttcagggctgcacagtcgtctcaagccttaaagacgttcttgatatttgcagcggaccggaagagtgttttgtcattggcggagcgcaattatacacagatctttttccgtacgcggatagactgtatatgacaaaaatccaccatgaatttgaaggcgacagacactttcctgaatttgacgagagcaactggaaactcgtgtctagcgaacagggcacgaaggatgagaaaaatccgtatgactatgaatttcttatgtatgaaaagaaaaacagcagcaaagcgggaggcttttgaaaaagaggggagggaaacattaatggcggggcttcttttatcgaatccagcgtga

SEQ ID NO: 18 - DNA, 1460 bp, Artificial Sequence
  Assembly fragment
gccttttaatcccggcaacagcttaatcagtacatccatcattccgaagcatccgacattcgatcattacaaggaattatttgcgggcaaggaaagccttcaatatgtgcagtggtatgtcaactctatgaagatcagcctgtttacaatggcagggtctttgctctgtgtgacgtttacggcctatgcgttttcgcgctttcggtttaaagggaggaaatacgctttaacgctctttttattgctgcagatgattcctcagttttcagctttaattgccttgtttgtgctggcgcaaatcttgggaatgatcaatagccactggctgctaatcttgctttatatcggcggcctgatcccgatgaatacgtatttgatgaaagggtacatggattccattccgatggatttagacgaaagcgccaagattgacggagccagcagcaccagaatcttcttccagatcattctgccattatcaaaaccgatggcggcagtcgtggccatgaacggctttaccggtccgctcggagattttgtgctgtcctcaaccatattgagaacgcctgaatcatatacattgcccgtcggtctattcaatttagtgaatgatgtcatgggggccagctatacgacatttgcggccggagccctgcttatcagcataccggttgccgtcatctttattatgctgcaaaagaattttgtgtccggattaaccgcaggcggaacgaagggctaagagaacaaggaggagaatgtgatgtcaaagcttgaaaaaacgcacgtaacaaaagcaaaatttatgctccatgggggagactacaaccccgatcagtggctggatcggcccgatattttagctgacgatatcaaactgatgaagctttctcatacgaatacgttttctgtcggcatttttgcatggagcgcacttgagccggaggagggcgtatatcaatttgaatggctggatgatatttttgagcggattcacagtataggcggccgggtcatattagcaacgccgagcggagcccgtccggcctggctgtcgcaaacctatccggaagttttgcgcgtcaatgcctcccgcgtcaaacagctgcacggcggaaggcacaaccactgcctcacatctaaagtctaccgagaaaaaacacggcacatcaaccgctacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagttggcttcgttgggattctccggtgatgctgatttcgctgcgtatgctgcg

SEQ ID NO: 19 - DNA, 1038 bp, Artificial Sequence
  Synthetized DNA fragment for erythromycin resistance cassette
tctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcc

SEQ ID NO: 20 - DNA, 5390 bp, Artificial Sequence
  Assembly of artificial folate operon as an expression cassette
tcccggcaacagcttaatcagtacatccatcattccgaagcatccgacattcgatcattacaaggaattatttgcgggcaaggaaagccttcaatatgtgcagtggtatgtcaactctatgaagatcagcctgtttacaatggcagggtctttgctctgtgtgacgtttacggcctatgcgttttcgcgctttcggtttaaagggaggaaatacgctttaacgctctttttattgctgcagatgattcctcagttttcagctttaattgccttgtttgtgctggcgcaaatcttgggaatgatcaatagccactggctgctaatcttgctttatatcggcggcctgatcccgatgaatacgtatttgatgaaagggtacatggattccattccgatggatttagacgaaagcgccaagattgacggagccagcagcaccagaatcttcttccagatcattctgccattatcaaaaccgatggcggcagtcgtggccatgaacggctttaccggtccgctcggagattttgtgctgtcctcaaccatattgagaacgcctgaatcatatacattgcccgtcggtctattcaatttagtgaatgatgtcatgggggccagctatacgacatttgcggccggagccctgcttatcagcataccggttgccgtcatctttattatgctgcaaaagaattttgtgtccggattaaccgcaggcggaacgaagggctaagagaacaaggaggagaatgtgatgtcaaagcttgaaaaaacgcacgtaacaaaagcaaaatttatgctccatgggggagactacaaccccgatcagtggctggatcggcccgatattttagctgacgatatcaaactgatgaagctttctcatacgaatacgttttctgtcggcatttttgcatggagcgcacttgagccggaggagggcgtatatcaatttgaatggctggatgatatttttgagcggattcacagtataggcggccgggtcatattagcaacgccgagcggagcccgtccggcctggctgtcgcaaacctatccggaagttttgcgcgtcaatgcctcccgcgtcaaacagctgcacggcggaaggcacaaccactgcctcacatctaaagtctaccgagaaaaaacacggcacatcaaccgctacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagttggcttcgttgggattctccggtgatgctgatttcgctgcgtatgctgcgatgaaagaagtcaataaagaacaaattgaacaggcagtgagacagattcttgaagcaattggagaagatccgaacagagagggcttacttgatacaccgaaaagagttgctaaaatgtatgcggaagtcttttcaggcttaaatgaagatccgaaagagcattttcagacaattttcggagaaaaccatgaagagctggtccttgtgaaagatattgcgtttcactcaatgtgcgaacatcacctggtgccgttttacggcaaggcacacgttgcgtatattcctagaggcggaaaagttacaggcttgtcaaaattagcacgcgcagttgaagctgttgcaaaaagaccgcaattacaggaacgcattacatctacaattgcggaatcaattgtcgagacattagaccctcatggcgttatggttgtcgttgaagctgaacacatgtgcatgacaatgcgcggcgtcagaaaacctggcgcaaaaacagtcacatcagcagtcagaggcgtgtttaaagatgatgcggcagctcgtgcggaagtcctggaacatattaaacgccaggactgaaaaagaggggagggaaacattatgatgatttcatttattttcgcaatggacgcgaatagactgataggcaaagacaatgatctgccgtggcatttaccgaatgacctggcttattttaaaaaaattacaagcggccatagcatcattatgggacgtaaaacatttgagtcaattggcagacctcttccgaacagaaaaaacattgttgtcacatctgcgccggattcagaatttcagggctgcacagtcgtctcaagccttaaagacgttcttgatatttgcagcggaccggaagagtgttttgtcattggcggagcgcaattatacacagatctttttccgtacgcggatagactgtatatgacaaaaatccaccatgaatttgaaggcgacagacactttcctgaatttgacgagagcaactggaaactcgtgtctagcgaacagggcacgaaggatgagaaaaatccgtatgactatgaatttcttatgtatgaaaagaaaaacagcagcaaagcgggaggcttttgaaaaagaggggagggaaacattatgatgaacaacattgcgtacattgcgcttggctctaatattggagatagagaaacgtatctgcgccaggccgttgcgttactgcatcaacatgctgcggtcacagttacaaaagtcagctcaatttatgaaacagatccggtcggctatgaagaccaagcccagtttttaaatatggcggttgaaattaaaacaagcctgaatccgtttgaacttctggaactgacacagcaaatcgaaaacgaactgggccgcacacgcgaagttagatggggcccgagaacagcggatttagacattctgctgtttaacagagaaaacattgaaacagagcagttaattgtcccgcatcctcgcatgtatgaacgcctgtttgttcttgcgccgcttgcggaaatttgccagcaggtcgagaaagaagcgacaagcgcggaaacggatcaagaaggagttagagtttggaaacaaaaatcaggcgttgacgaatttgtacatagcgaaagctgaaaaagaggggagggaaacattatgatggcgcagcacacaatagatcaaacacaagtcattcatacgaaaccgagcgcgctttcatataaagaaaaaacactggtcatgggcattcttaacgttacacctgattcttttagcgatggtggaaaatatgacagcttggacaaggcgcttctgcatgccaaagaaatgatcgacgacggcgcgcacattattgacataggaggcgagagcacaagaccgggagctgaatgcgtcagcgaagacgaagaaatgtctcgggtcattccggtcattgaacgcatcacaaaggaactcggcgtcccgatttcagtggatacatataaagcatctgtggcagacgaagcagtcaaagcgggcgcatctattatcaatgacatttggggagcgaaacatgatccgaagatggcaagcgtcgcagcggaacataacgttccaattgtcctgatgcacaatcggccagaacggaattataacgaccttcttccggatatgctgagcgaccttatggaatcagtcaaaattgcggttgaggcgggcgtggatgagaaaaatattattttagatccgggcatcggcttcgcgaagacataccatgataatcttgcagtgatgaataagttagagatcttcagcggacttggctatcctgtcctgctggctacatctcgtaaaagatttatcggaagagttcttgatttaccgcctgaagagagagcagagggcacaggagcgacagtctgcttgggcattcagaaaggatgcgacatagtgcgtgttcatgatgtcaagcaaattgccagaatggcgaaaatgatggacgcgatgctgaataagggaggggtgcaccatggatgaaaaagaggggagggaaacattatgatggataaagtttatgtggaaggaatggaattttatggctatcatggcgtcttcacagaagagaacaaattgggacaacgcttcaaagtagatctgacagcagaactggatttatcaaaagcaggacaaacagacgaccttgaacagacaattaactatgcagagctttaccatgtctgtaaagacattgtcgaaggcgagccggtcaaattggtagagacccttgctgagcggatagctggcacagttttaggtaaatttcagccggttcaacaatgtacggtgaaagttattaaaccagatccgccgattcctggccactataaatcagtagcaattgaaattacgagaaaaaagtcataaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagat

SEQ ID NO: 21 - DNA, 2124 bp, Artificial Sequence
  Tetracycline resistance cassette
aattcttactgcagatagtgtacgtaaaaagattaaattattgcttggtgaaaaaagtcttgcaatggtgcaggttgttctcaatgtcgaaaatatgtatttgtatttaacgcacgagagcaaggacgctattgctaagaagaaacatgtttatgataaggctgatataaagctaatcaataattttgatattgaccgttatgtgacgttagatgtcgaggaaaagaccgaacttttcaatgtggttgtatcgcttattcgtgcgtacactctccaaaatatttttgatttgtatgatttcattgacgaaaatggagaaacttatgggttgactataaatttggttaacgaagttattgcagggaaaactggttttatgaaattgttgtttgacggagcttatcaacgtagtaagcgtggaacaaagaacgaagagagataaaaagttgatctttgtgaaaactacagaaagtaaagaatgaaaagagtaatgctaacatagcattacggattttatgaccgatgatgaagaaaagaatttgaaacttagtttatatgtggtaaaatgttttaatcaagtttaggaggaattaattatgaagtgtaattaatgtaacagggttcaattaaaagagggaagcgtatcattaaccctataaactacgtctgccctcattattggagggtgaaatgtgaatacatcctattcacaatcgaatttacgacacaaccaaattttaatttggctttgcattttatctttttttagcgtattaaatgaaatggttttgaacgtctcattacctgatattgcaaatgattttaataaaccacctgcgagtacaaactgggtgaacacagcctttatgttaaccttttccattggaacagctgtatatggaaagctatctgatcaattaggcatcaaaaggttactcctatttggaattataataaattgtttcgggtcggtaattgggtttgttggccattctttcttttccttacttattatggctcgttttattcaaggggctggtgcagctgcatttccagcactcgtaatggttgtagttgcgcgctatattccaaaggaaaataggggtaaagcatttggtcttattggatcgatagtagccatgggagaaggagtcggtccagcgattggtggaatgatagcccattatattcattggtcctatcttctactcattcctatgataacaattatcactgttccgtttcttatgaaattattaaagaaagaagtaaggataaaaggtcattttgatatcaaaggaattatactaatgtctgtaggcattgtattttttatgttgtttacaacatcatatagcatttcttttcttatcgttagcgtgctgtcattcctgatatttgtaaaacatatcaggaaagtaacagatccttttgttgatcccggattagggaaaaatataccttttatgattggagttctttgtgggggaattatatttggaacagtagcagggtttgtctctatggttccttatatgatgaaagatgttcaccagctaagtactgccgaaatcggaagtgtaattattttccctggaacaatgagtgtcattattttcggctacattggtgggatacttgttgatagaagaggtcctttatacgtgttaaacatcggagttacatttctttctgttagctttttaactgcttcctttcttttagaaacaacatcatggttcatgacaattataatcgtatttgttttaggtgggctttcgttcaccaaaacagttatatcaacaattgtttcaagtagcttgaaacagcaggaagctggtgctggaatgagtttgcttaactttaccagctttttatcagagggaacaggtattgcaattgtaggtggtttattatccatacccttacttgatcaaaggttgttacctatggaagttgatcagtcaacttatctgtatagtaatttgttattacttttttcaggaatcattgtcattagttggctggttaccttgaatgtatataaacattctcaaagggatttctaaatcgttaagggatcaactttgggagagagttcaaaattgatcctttctgcagaag

SEQ ID NO: 22 - protein, 419 aa, Lactobacillus reuteri
MRTYEQINAGFNRQMLGGQRDRVKFLRRILTRLGNPDQRFKIIHIAGTNGKGSTGTMLEQGLQNAGYRVGYFSSPALVDDREQIKVNDHLISKKDFAMTYQKITEHLPADLLPDDITIFEWWTLIMLQYFADQKVDWAVIECGLGGQDDATNIISAPFISVITHIALDHTRILGPTIAKIAQAKAGIIKTGTKQVFLAPHQEKDALTIIREKAQQQKVGLTQADAQSIVDGKAILKVNHKIYKVPFNLLGTFQSENLGTVVSVFNFLYQRRLVTSWQPLLSTLATVKIAGRMQKIADHPPIILDGAHNPDAAKQLTKTISKLPHNKVIMVLGFLADKNISQMVKIYQQMADEIIITTPDHPTRALDASALKSVLPQAIIANNPRQGLVVAKKIAEPNDLIIVTGSFYTIKDIEANLDEK

SEQ ID NO: 23 - protein, 406 aa, Ashbya gossypii
MELGLGRITQVLRQLHSPHERMRVLHVAGTNGKGSVCAYLAAVLRAGGERVGRFTSPHLVHPRDAITVDGEVIGAATYAALKAEVVAAGTCTEFEAQTAVALTHFARLECTWCVVEVGVGGRLDATNVVPGGRKLCAITKVGLDHQALLGGTLAVVAREKAGIVVPGVRFVAVDGTNAPSVLAEVRAAAAKVGAEVHETGGAPVCTVSWGAVAASALPLAGAYQVQNAGVALALLDHLQQLGEISVSHAALERGLKAVEWPGRLQQVEYDLGGVHVPLLFDGAHNPCAAEELARFLNERYRGPGGSPLIYVLAVTCGKEIDALLAPLLKPHDRVFATSFGAVESMPWVAAMASEDVAAAARRYTAHVSAVADPLDALRAAAAARGDANLVVCGSLYLVGELLRREH

SEQ ID NO: 24 - DNA, 1399 bp, Lactobacillus reuteri
ttttactagtatgagaacatacgaacaaattaatgcaggatttaatcgccagatgctgggcggccagagagacagagtcaagttccttagacgcatccttacgagacttggaaaccctgatcagcgctttaaaattattcatatcgcgggaacgaacggcaaaggatcaacaggcactatgttagaacagggccttcagaatgcgggataccgcgtcggctactttagctctcctgcgctggttgatgatcgcgaacaaattaaagtcaatgatcaccttatcagcaagaaagattttgcgatgacctatcagaaaattacggagcatctgcctgctgaccttctgcctgatgatattacaatctttgagtggtggacgttaatcatgcttcaatactttgcggatcaaaaggttgactgggcggtgattgaatgtggtcttggcggccaagacgatgcgacaaacatcatctcagcgccgttcatttcagtcattacccatatcgctcttgaccacacccgtatcctgggccctacaattgcgaagattgcgcaagctaaggcaggcattataaagacagggactaaacaggttttcctggcaccacatcaagagaaggatgcgttaacaatcattcgcgaaaaagcgcaacagcaaaaggtcggactgacgcaggcagatgcacagagcattgtggacggaaaagctattttaaaagtgaatcacaagatttacaaggtcccttttaatctgctgggcacatttcagtcagaaaacctgggaacggttgttagcgtctttaactttctgtatcagcgccgtcttgtcacgtcatggcaaccgttacttagcacactggcaacagttaaaattgcaggaagaatgcaaaaaatcgcggatcatcctccgatcattcttgatggcgcacataatccggatgctgcaaagcagcttacaaagacaattagcaaactcccacataataaagtcataatggtgttaggcttccttgctgacaaaaacatttcacagatggtcaagatttaccaacagatggcggatgaaattatcattacaacgcctgaccatcctacaagagcgctggacgcctcagcccttaaatcagtcttaccgcaagcaattattgcgaataatcctcgtcagggactggttgttgctaagaaaattgcagagccgaacgatcttatcatcgtcacgggcagcttctacacaatcaaggatattgaggcaaatttagatgagaaataagcagaggctgtgatcagtctctgcttttttttctgcgttctatttctttttcacgttcacggatgacgtcagtccgatcccgcaaacggtgtttgtcgataagaaatatgaattcgcgtgcgcattgga

SEQ ID NO: 25 - DNA, 1360 bp, Ashbya gossypii
ttttactagtatggagttaggcttaggccgcatcacacaagtgctgagacaattacatagccctcatgaaagaatgcgtgtcttacatgttgcaggaacaaatggcaaaggaagcgtctgtgcgtatttagcggctgttttaagagcgggcggagaaagagttggcagatttacaagccctcacttagttcatccgcgcgatgctatcacagtcgacggcgaagttattggagcggcgacatatgctgcacttaaagctgaagtcgttgcggcaggcacatgcacggagtttgaagcacaaacggcggttgcgcttacgcattttgcaagacttgaatgcacatggtgtgtcgtcgaagtgggcgtcggcggcagattagacgctacaaatgtcgtccctggcggacgcaaactgtgtgcaattacaaaggttggattagatcatcaggcgttacttggcggaacactggctgttgttgcaagagagaaggccggcattgtggttccgggagtgcgctttgtcgctgtcgacggcacgaacgcaccttcagttctggcggaggttcgggcggctgcagcgaaagttggcgcagaggtccatgagacaggaggcgcgccggtttgcacagtcagctggggtgcggttgctgcaagcgcacttccgttagcgggagcttaccaggtacaaaacgcgggcgttgcacttgcactgcttgatcatcttcaacaactgggagagatctcagtcagccatgcagcactggaaagaggactgaaagcagtcgaatggcctggcagacttcaacaagttgagtatgaccttggaggcgtccatgtcccgctgttatttgacggagcacacaatccgtgtgcagcggaagagcttgcaagattcttaaacgagagataccgcggaccgggaggatctccgctgatctatgtgctggctgtcacgtgtggcaaagagatcgacgcacttcttgcacctcttctgaaaccgcacgatagagtcttcgcaaccagctttggcgcggttgagtctatgccgtgggtcgcagcgatggcaagcgaggatgtcgcagcggcggcgagacgctacacagcccacgtttcagcggttgcggacccgctggacgcgttacgcgccgcagcggcagcacgcggcgatgctaatctggtcgtctgcggatcattatatcttgtcggcgaacttctgcgccgcgaacattaagcagaggctgtgatcagtctctgcttttttttctgcgttctatttctttttcacgttcacggatgacgtcagtccgatcccgcaaacggtgtttgtcgataagaaatatgaattcgcgtgcgcattgga

SEQ ID NO: 26 - DNA, 20 bp, Artificial Sequence
  Primer
gcagcgaaatcagcatcacc

SEQ ID NO: 27 - DNA, 20 bp, Artificial Sequence
  Primer
gactcgttagccaggtcgtc

SEQ ID NO: 28 - DNA, 20 bp, Artificial Sequence
  Primer
tcgataaaagaagccccgcc

SEQ ID NO: 29 - DNA, 20 bp, Artificial Sequence
  Primer
ggtttccatgagggtcggtc

SEQ ID NO: 30 - DNA, 20 bp, Artificial Sequence
  Primer
gctacctggcgcaaaaagaa

SEQ ID NO: 31 - DNA, 20 bp, Artificial Sequence
  Primer
cggtagtcattgctggcgaa

SEQ ID NO: 32 - DNA, 19 bp, Artificial Sequence
  Primer
acgacctggctaacgagtc

SEQ ID NO: 33 - DNA, 20 bp, Artificial Sequence
  Primer
ggcggggcttcttttatcga

SEQ ID NO: 34 - DNA, 20 bp, Artificial Sequence
  Primer
gaccgaccctcatggaaacc

SEQ ID NO: 35 - DNA, 20 bp, Artificial Sequence
  Primer
ttctttttgcgccaggtagc

SEQ ID NO: 36 - DNA, 20 bp, Artificial Sequence
  Primer
ggagaatcccaacgaagcca

SEQ ID NO: 37 - DNA, 152 bp, Artificial Sequence
  Fragment containing the Pveg promotor
gcatcactatctgcagtaaaatcggaattcaattttgtcaaaataattttattgacaacgtcttattaacgttgatataatttaaattttatttgacaaaaatgggctcgtgttgtacaataaatgttactagagtaaaggaggaaactagt

SEQ ID NO: 38 - DNA, 228 bp, Artificial Sequence
  p15
gtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagt

SEQ ID NO: 39 - DNA, 25 bp, Artificial Sequence
  Primer
atctacattccctttagtaacgtgt

SEQ ID NO: 40 - DNA, 32 bp, Artificial Sequence
  Primer
aaatctagaaattaagaaggagggattcgtca

SEQ ID NO: 41 - DNA, 34 bp, Artificial Sequence
  Primer
aaaggatccatctacattccctttagtaacgtgt

SEQ ID NO: 42 - DNA, 20 bp, Artificial Sequence
  Primer
tcccggcaacagcttaatca

SEQ ID NO: 43 - DNA, 20 bp, Artificial Sequence
  Primer
ggagccgattctctgcgtta

SEQ ID NO: 44 - DNA, 20 bp, Artificial Sequence
  Primer
aaaatgctccctgcggctat

SEQ ID NO: 45 - DNA, 21 bp, Artificial Sequence
  Primer
caatgagaggggttgctatga

SEQ ID NO: 46 - DNA, 20 bp, Artificial Sequence
  Primer
tcgaacggtcaagcacgtta

SEQ ID NO: 47 - DNA, 34 bp, Artificial Sequence
  Primer
tttgctagcatgataattggaatatgggcagaag

SEQ ID NO: 48 - DNA, 34 bp, Artificial Sequence
  Primer
tttgcggccgccctcctcgtcatttcttcaaaag

SEQ ID NO: 49 - DNA, 6975 bp, Lactococcus lactis
atgataattggaatatgggcagaagatgaagcaggtcttatcggtgaagctgataaaatgccttggtctttacctgctgaacaacaacattttaaagaaacaaccatgaatcaagtgattttgatgggacgaaaaacgtttgaaggcatgaataaacgtgtattgccagggagaataagtattattttaactcgcgatgaaacttatcaatcagataatgaaaaagtgctcatcatgcacagccctaaggaagttctagattggtaccataagcaaaataaagacttatttatcacaggaggagctgaaattttagccctttttgaatctgaacttgaattgctctatcgaacagttgttcatgaaaaatttaaaggagatacttattttccaagtacatttgactttggaagatttaagctagtctctgaaaaatttcacgataaagatgagcggaattcttatacttttacaattaaaaaatatgaaaaagtgaaacaaccatgacaaaatcaatttttgggcttttcacagctctcctttgttggattagcattgtcatcgctattcaatgctttagaaaaaaacgttggggtctgggagtattgtttttactcaatgcttttacgaacctcgtaaatacaattcacgctttttctggaactttattttaaaaaataaaaaaagtgccttttaagtaagccaataacacttactttttatgttagtgaaatcaggaataaaataactatgtcaaatacacaaaatccaaatatacattgttctttctgtggaaagagtcaagatgatgtaaaaaaattgattgccggttcagacgtttatatttgtaatgaatgtattgaactttcaactcgaatcttagaagaagaattaagagaagaacaagattcagaaatgcttgaagttaaaacacctaaagaaatgtttgaccatttaaatgaatatgtgataggtcaagaaaaagcaaaacgtgcacttgcagttgccgtttataatcattacaaacgaattaattttgcagcaagtaaaattgctgaagatattgaactacaaaaatcaaatattctattaatcggacctaccggttctggtaagacttttctcgctcaaactttagcgaaatcactcaacgttccatttgcgattgcagatgcgacaagtttaactgaagctggttatgttggagaagacgttgaaaatattctcttaaaacttttacaagcgagtgatttcaatattgaacgtgctgaacgtggaattatctatatcgatgaaattgataaaattgctaaaaaatctgaaaatgtatcaattactcgtgacgtttccggggaaggtgttcaacaagcccttttgaaaattattgaaggaacggtagctagtgttccaccacaaggtggacgtaaacatcctaatcaagaaatgattcaaattgataccaaaaatatcttatttattgttggtggagcttttgacgggattgaagaaattgtcaaacaacgtttaggtgaaaaaattattggttttggtgccaataataaaaaattaaatgacgatgattcttatatgcaagaaattattgccgaggacattcaaaaattcggattaattcctgaatttattggtcgtctgccaattgttgctgctttggaacgtttgaccgaagaggatttgattcaaattttgacagaacctaaaaatgctttgattaaacaatataaacaactccttttatttgataatgttgaacttgaatttgaagatgaagccctcatggcaattgctagaaaagcaattgagcgcaaaacaggagcgcgtggacttcgttcaattattgaagaagtaatgatggatatcatgtttgaagttccaagtcatgaagaaattacaaaagttattattaatgaagcagttgttgacggaaaagctgagccacaaatgattcgagaggccaagaaaaaatgaccataaatacaaataatctgacaataacaatttcagcagcatcaaaaaaacaatatccagaaaatgattggccagaaattgccttagctgggcgttcaaatgtcggtaaatcaagttttattaatactttacttaatcgtaaaaactttgccagaacttctggtcaacctggtaaaacacagttgctcaatttttataatattgatgatcaacttcatttcgttgacgtacctggttacggctacgctcgtgtttctaaaaaggaacgcgaaaaatggggtaaaatgattgaggaatatttgacaacaagagaaaatttaaaagcagttgtcagcttagttgatattcgtcatgaaccctcagaagatgatttgatgatgtatgagtttttgaaatactaccatattccagtgattttagttgcgaccaaagccgataaagttccacgtggtaagtggaataaacatgaatctattatcaaaaaagcaatgaaatttgatagtacagatgattttattatcttttcttctactgataagacaggatttgaagaagcttgggaagcgattttaagatatctctgaaaatagtgctatgaagagattcatagccttttctacacttaaaaagaggaaatatgtacaaaataaaacttaataatataaaatttagggcacatattggtgttctgccagaagaaaaagttctcggacaaaatctcgaaattgatttaatcgtggaaacaaattttgatttttcaggaaaagacgaattagatgaaactttgtcttatgttgatttctatgaggcaacaaaagcagttgtagaatcttcaaaagctgatttaattgaacatgttgcctttgaaattattcaagcagtaaaggctacttcagagcgtatatcaacggttgaagtccatcttagaaaattagccgtaccgattgaaggaatttttgattcagctgaaattgagatgagaggctaaagctggtttttaagataaatattttaaagagatagaagagaaacaaaatcataaaagattatgtctaaatggaggacttatgcaaacaacttacttaagcatgggaagtaatattggtgaccgtcagtattatttacatgaagccattcgtttattgggaaaacaccctaaaattatgattgaaaaagtatcaaatttttatgaaagtactccagtcggcggcgtcaaacaagatgattttactaatttggcattaaaggtggcaacgctacttgaacctttggaattattatcttttattcatgaagttgagttatctttgaaccgtgagcgaaaaattcattgggggccaagaacaattgatattgatattattttctatgacgacttagaaatgcaagtagaaaacttggttattccacataaagaagcttttaatcgtctttttgtcttgaaacctatttttgaacttattgataaagactttaaatattatgcgtcaatagaaaaagcaatagccgaactttcagtaagtgaacaagagctccatgtaataaaagaagaaaaaacaccgagaaatcgtattgaagatgccgttaaagagattctctttgcagtaggtgaaaatccaaatcgagaaggattacttgaaactccagcaagagtagctaaaatgtatgaagaaattctttcgtcacaacgcttaagcaagtttaatgagtataaactttttgaaattgattcttctaaaacggattcaatcgtgttgattaaagatattcctttttattcaatgtgtgagcatcatatgttaccattttttgggaaagctcatgttgcatatattccagctgatggaaaaattattggcttgtcaaaaattccccgtttagttgattatgtttcgcgcaaactctcggttcaagaaaatatcactcatgatattggagatattttgactgatattttgaatcctaaaggagtggcagttcttgttgaaggacgtcatatgtgcgttgaaatgcgtggagtaaaaaaagtaaattctattactaaaacttcttattttttaggtgaatttaaagaaaataatgaaaaaagaatggaatttttagaaagtcttttatgaaaatcttagaacttaatcaagaatctttttctcttaaaaatattatcctaaaatttgatgagttaaatcacaatgaaatgatttctcttcaaaaaaaactttatcgaaatggtagtttgacaagactggctccagactccttgttagtagttttaacaattgatgacttagcaaaattgattaatctttttgaaaatgatgaagataaaaaaatgcttgaagtgatttataagcgtcatcaaatcatttggtcaggtaaaaatttcaattttgatttaactagaaagtcaattgtctattcaatcgtcaatgttacaccagactctttttatgatggaaatccagataatttaaacctctctcatattttaaaaagagtagaagctgatttagaaaatggagcttctgttcttgagctgggagggaaatcatcgaaaccaggatatgacgatattagcccagaagaggaatggaacagactgaaagaacctattcttgagttgaaaaaaaactttcctaaagcgatttttgctgtcgatacggatgaagcttatgtcatggaacgagttttagacgctggggttgatattattaacgatattgatggttttgatacaaatgataaattaaaagtggtagaaaagtatcaaccggctttagttgctatgaataatgggcgagctggttttagttatgctgataatgtttatgaagaacttccattattttttgaaaataaaaaagaagagttacttcaacttggtttaaaagctgagcaaatcgttattgatcctggagttggtttttttaatggagattcaggttcagatagtcttgagcgggttaaagcaactgaaattttaagcagaataggtttacctcttatgattgcaatctctcgtaagtcatttatgggaaaactcttcaatgcccaaggagatgagcggcttttttcaagccttgtcctagaagcgcaaatggttgctgatgggggacggattttgcgtgttcatgatgttaaggagactaaacgtttactcgatgcaattgaaatttataaggaattttaaaaatgaatgaagacctaattgctgaaattcaagctttatctgctattggaagtgaagaaaaattttccgagattattcgattattgaaaaattcgactttagagcttcgggggaaaaagaatccagatttacaattgtcagcaagtgcattagtttttaaaaaacataaactattttttattgaacacccttatcaaaaggagcttttgcttccagcaggtcatgttgaactaggagaaaagccattggaaactgcgattcgtgagttccatgaagaaacaggtttttcagcgtcagaatcaggcaagttggtagatgttaacttgattaatattccttacaacaaaattaagaatgagaaagaacatcaacacattgattttcgttttctattggaactaaaagaaaaagaagcaggccttgctgaattgccttttttccttcttgatagaactgaagctcctgatgaatttaaaaaatattatcaatacaaaagataaagtagaaaaggtcacaaaatgtctatagaagaagcattggaatggatacattcacgtttaaaatttaatattcgcccaggcctaagtcgtgtttcggcccttttagaattgcttggtcatccagaagagtctttgtcaatgattcacgttgctggaacaaatggaaaaggctccacagtcgctttcacacgctcaatctttatgcaggcaggtctgaaggttgcttctttcacaagtcctttcatcaccacttttggtgagcggatgtcgattaatgcactcccgattgctgatgataaattaatttattatgtagaaatgatccaaccacttgttgctgaacttgataaagatgctgaactgactggaattaccgaatttgaaattatcacggcaatggcttttaaatatttctctgatgagcaggttgatttagcggttattgaagttggtttaggtggacttcttgattcaacaaatgtgattaaacctgttgtttctggaattacaacaattggtttagatcatattgatattcttggttcgaccattgaagaaatcgcagctcaaaaggctggaattattaaaccaggaattccagtagttgttggaaatattgaattaaaagcacttcgggttatatgggaagtggctagaaaaaatacagcgcgtgtttatcaatttccatatgattatcgtacggaagtggaagaacacgaacattttaatttcttttctggtcaagaagcaatattggatattgaaaaatctttagttggcttacatcaaatagaaaatgctggtatggctattgaactttctctggtttatgcaagtaaggttgggattgaattgactgaggatgtgattcgctctggaattcgtgaggctttttggccagctcgtatggaaaaattgggtgaaaaaccactcattttactggatggtgctcataatgttcatgcgatgaatcgtttgcttgaaaatcttagctctgagtttccagataaaaaaattacaatcattttttcagccattaccacaaaagatattagtcaaatgataaaaatgcttcaaactgtgaaaaattcgcatctgattttgacaacttttgattatccaaaagctttgaatttgggagattttcaaagattggaagaagaaggggttgaattggctccaagttgggaattagctttagttcgtgcgcaaaaaaatttagctgaagatgatttgttattagttacaggctctctctatttctcatctcaagttcgtgagtttttgaaaaaagagaagtaa

SEQ ID NO: 50 - DNA, 2463 bp, Artificial Sequence
  Synthetic folate biosynthesis gene fol1-AG
atgcagtcccttggattcaagtgtttgctgtctcgcaggagcctgagcaggatatcaatctgtacaagaggaatgagtagtgctaacggtggacgaagtaatgatactgtgcatatacagagacaggcactgaaagttgttgctgggcttgacggatggggtcaattgcaggcgcaggatgtgaaattgaccatgaatatgaacacagattttcgtgcttcctcgcagacggatgatctgaagtactccttgaattatgcggtgatttcacgtggggtgcataggttcgttgagggctgtggacggtaccgctctcttggtcacttggccagggaggtaaagaagttttccatgaatgagtatccgggtatccaaactatagaggtgggtgcggaggcggacgcggcccatttgcgatgcggaagtctgggcgtcgtggtgaacagcgatgggcatcgtcctgatgagattttgctttctggaatgaagcttctgacactaataggggtgttcacttttgaacggcgtcggaagcagtacgttgacttgaagctgtcatttccgtggccgaaggaggctggtgaatttccggattgccaggaattattggacgatgttgtgagctatgtagagagagcgaattttaaaacggcagagtctcttgctgagagtgtagctcacgttgttaccttgagagagtattttcagctgcatcgtgggttaccggtaaaagtcaaggtaattaagcttaatgccattactgagactgagggagttggtgtgagctgtgtaagaagtgcggatgaatttacggggaaaccgcccttctgggaagatattccaaacgatcgagcagacgtgtttaaccttcctgtattccagcagccacatgcatctgtcagtgagtggaatcgtgtgtttctggcgtttggatctaatataggggataggtttgctcacattgagcgaagcttacgtctacttgcggaagatcctaaagttaaactacttcgctcgtcgtctctgttcgagagtgaaccaatgtactttaaggagcagtccccgtttatgaatggcgttgtagaagtgcagacacggtatagcccgcacgagttactagagctatgcaaaaggatagaatatgaacatttaaaacgtgtcaaagagtttgataacggccctcgcagcattgatttagatattttattgtaccaaaatgcaaactttgagcatgtggtactgaactccgaggatttagttattcctcatccaaggatgttggagagatcgtttgttttagagcctctctgtgaattgttggctttccatgaagtgcaccccatttcggctgaatctgtccaaagtcacctaaaagaattgtaccgtaaggggaataaggaagacattcttgttaaacttgtacctttgccgggtattccgtcaaatatacctacaacgcgatttctgaagtttagacgggagtatgaggaggatcaatcgacaagcgaattggttcttaggaccaagtcaaatacatatgtcatgggcatcgtgaatgtgacacctgattctttttctgatggatctcctatgtggaatgatgttaatcatttcctcttaaaagtacaaaggatgatccttgacgttttgaagttacatgaaaacgttatcattgatattggaggctgttcgactaggcctggtagtcagcaaccatcagtggaagaagaacttagtcgtactattcccctaataacagcgatcaggggttgcagagatttttcgcaagagaatgtgatcatatctatagacacttacagaagtgctgttgctgaaaaggccataacagcaggggctgatattgtgaacgatatttcaggaggtagttttgatacaaatatgtttaaggttatcagcgcgtatccgaatgttggttatgtgctatcacacataaggggagatatgactaccatgacgagcctgaataagtatgatgatacagttggtttggatggcgttgaagaattcatttacggtaagaaacagcactcagaacggactaaggtgatccggaacatttgtagggaacttgcggagcgataccagcttgcccttgctagcggaattaagcgctggcagattattttggatccgggtattggttttgcgaagaatgctaaacagaacttagatatcatcaagcataccccgtcaattaagggttatagttgtgtgacacatggacaatttgtaaattttgccaaccttcctgtgttgcttgggccttccaggaagaactttattgggactataattcaagaggcacaggtcgagcgaagggactttgcaacggggactattgtaggctcctgtgttggttatgatgcggatatcatcagggtacatgatgtaactaactgtagcaaaagtgctaggttagcggatgagctttataggaaatag

SEQ ID NO: 51 - DNA, 732 bp, Artificial Sequence
  Synthetic folate biosynthesis gene fol2-AG
atgtgtcaggggggcagtaaaggactagttaggcaggacacgcccctaaagacgaggcctgtctcgccatatacgctccaggccccagttgaggcggacggactgtcctggccgagtgcaggggcacgtgtgcgggtcgaggagggcacggaggaagaggcggcacgcgcagcccggatagctgatgcagtcaagacgattttgacggagctgggcgaagacgtgacgcgggagggcctgctggacaccccgcaacggtacgccaaagcgatgctgtacttcaccaagggctaccaagacaatattttgaacgatgtgatcaataatgctgtgtttgacgaagatcatgacgagatggtaattgtgcgggatattgagatccattcgctgtgcgagcaccacctggtacccttcttcgggaaggtgcatattggctacatacctcggaggagagtcctcgggttgtcgaagctcgcccggctagcggaaatgtacgcgcgcaggctgcaggtgcaggagcggctgacgaagcagattgcgatggcattgcaggatatactgcgccctagaggagtagccgttgtggtggaggccacgcatatgtgcatggtgtcacggggggtccagaagtccgggtcctcaactgtcacctcgtgtatgctgggctgcttcagagacatgcacaagacccgggaagaattcttgaacctcttgagaaatagaagtgtatag

SEQ ID NO: 52 - DNA, 2484 bp, Artificial Sequence
  DNA fragments FOL1-AG
ttttactagtatgcaatcactgggctttaaatgtcttctgagcagaagaagcctgagccgcattagcatctgcacgagaggaatgagctcagcgaatggaggaagaagcaatgatacagttcatattcagcgccaggcacttaaggtcgttgcgggccttgatggctggggacagctgcaggcgcaagacgttaagctgacaatgaacatgaacacagactttcgtgcgtcaagccaaacagatgaccttaaatacagccttaattacgctgtgattagccgtggagtccaccgttttgtcgagggatgcggaagataccgtagcctgggacatctggcgagagaggtcaaaaagttttcaatgaatgagtaccctggcattcagaccattgaggtgggtgccgaggcggacgcggcacacctgagatgcggatctttaggcgttgttgtgaatagcgatggacacagacctgatgagatcttattgtcaggcatgaaacttctgacgctgattggagtctttacattcgagcgtcgcagaaagcaatacgtcgatctgaaactgagcttcccgtggcctaaagaagcaggagagttcccggattgtcaggaacttctggatgacgttgtgagctacgtcgagagagcgaacttcaaaacggcagagtctctggcggagtctgtggcacacgtggtcacacttcgcgaatattttcaacttcatcgtggcttgcctgtcaaagtgaaagtcattaagctgaacgcgatcacagaaacggagggcgtcggagttagctgtgtcagatctgccgatgaatttacaggcaagcctccattttgggaagacatcccgaacgatagagcggacgtctttaatttacctgtgttccagcaacctcatgcaagcgtttcagagtggaatagagtgtttctggcgtttggctccaacattggagatagattcgcgcatatcgagagatctttacgtctgcttgctgaagatcctaaagtcaaactgcttagaagcagcagccttttcgaatctgagcctatgtatttcaaggagcagtccccgtttatgaacggagtcgttgaggtccaaacgagatattcaccgcatgaacttttagagttgtgcaaacgtatcgaatatgaacacctgaaacgtgttaaagagtttgataatggcccgcgttcaattgacctggatatcttactgtaccagaacgcgaactttgagcatgtggtccttaattccgaagacctggtaattccgcatcctagaatgctggaacgcagcttcgtgctggagcctttatgcgagctgcttgcgtttcacgaggttcaccctatatcagccgagtcagtgcagagccatctgaaagaattatacagaaaaggcaataaagaggacattttagtcaagttagtccctctgcctggaatcccttctaatattccgacgacgagatttcttaaatttagacgcgaatatgaagaggaccagtctacatcagaattagtcctgcgtacgaaaagcaacacatacgttatgggaattgtcaatgttacgcctgactcatttagcgacggctcacctatgtggaacgacgtcaatcatttccttctgaaggtgcaacgcatgatcctggatgtcctgaaactgcatgagaatgtcattattgatatcggaggctgctctacaagacctggctctcagcaaccgagcgttgaagaagagttatcacgcacgattcctcttattacagctattcgcggctgcagagatttttcacaagagaatgttattatctcaattgacacataccggtcagcggtcgctgagaaagcaattacggcaggagcggatattgttaatgatatttctggcggatctttcgatacaaatatgtttaaagttatttcagcgtatcctaatgtcggctacgttctgtcccatatccgtggcgatatgacaacgatgacgtcactgaacaaatatgatgacacagtcggcttagatggcgttgaggaatttatctatggcaaaaaacaacattcagaacgtacaaaagtcatccgtaacatctgtcgcgaacttgcagaacgctaccagcttgcacttgcttcaggcattaaacgctggcaaattatccttgatcctggcattggcttcgctaaaaatgctaaacaaaacctggatattattaaacacacgccgagcattaaaggatactcatgtgtgacgcatggacaatttgtgaatttcgcgaatttaccggtactgctgggcccgtctcgcaagaatttcatcggcacaattattcaggaggcgcaagtagaacgcagagatttcgcaacaggcacgattgtgggctcatgtgtcggctatgacgctgatattatccgcgttcacgatgtcacgaattgtagcaagagtgcacgcctggcggatgaactgtatcgcaaataaggatccatttt

SEQ ID NO: 53 - DNA, 1090 bp, Artificial Sequence
  DNA fragments FOL2-AG
tattggatcctatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgtgtcaaggcggaagcaaaggactggttagacaagacacaccgctgaaaacaagacctgtctcaccttatacactgcaagcacctgtcgaagcagacggattaagctggccgagcgcgggcgcgagagttagagtggaagagggaacggaggaagaagcagcgcgcgcggctagaattgcggatgcagtcaaaacaatattaacagagctgggcgaagacgtgacaagagaaggtcttctggacacaccgcagcggtatgcgaaagctatgctgtactttacgaagggataccaagacaacatcctgaacgatgtcattaacaatgcggtttttgacgaggatcatgatgagatggttatcgttcgcgacatagagatacacagcctgtgtgagcatcacctggtcccatttttcggcaaggtccacataggctacattccgagaagacgtgtcctgggactttctaaactggcgcgcttagctgaaatgtacgcacgcagactccaggtccaagaacgtttaaccaaacagatcgcaatggcactgcaagatatccttcgccctagaggcgtggcagtcgttgttgaggctacgcacatgtgcatggtctctcgcggagtgcaaaagagcggatcatcaacggtaacatcatgtatgctgggatgtttcagagacatgcacaagacgagagaggaatttcttaatttacttagaaacagaagcgtttaagcagaggctgtgatcagtctctgcttttttttctgcgttctatttctttttcacgttcacggatgacgtcagtccgatcccgcaaacggtgtttgtcgataagaaatattacgtaatatggcctcgagtttta

SEQ ID NO: 54 - DNA, 25 bp, Artificial Sequence
  Primer
tattggatcctatggttcactgtcc

SEQ ID NO: 55 - DNA, 20 bp, Artificial Sequence
  Primer
gcggtagtggtgcttacgat

SEQ ID NO: 56 - DNA, 23 bp, Artificial Sequence
  Primer
tgcagggtctttattcttcaact

SEQ ID NO: 57 - DNA, 20 bp, Artificial Sequence
  Primer
gcggtagtggtgcttacgat

SEQ ID NO: 58 - DNA, 1221 bp, Ashbya gossypii
atggagctgggactcgggcggataacacaagtgctgcggcagcttcacagcccgcatgaaaggatgcgcgtgctgcacgtggccggtacgaacggtaagggctcggtctgtgcgtacctcgcggcagtgctgcgcgcgggcggagagcgagtgggaaggttcacgagcccgcacctggtgcacccgcgggatgcgatcacggtggatggggaggtgatcggggcggcgacgtacgcggccctgaaggcggaggtggtcgcggcggggacatgcacggagtttgaggcgcaaacggcggtggcgttgacgcactttgcgcggttagagtgcacgtggtgtgtggtggaggtgggcgtgggcgggcggctggacgcaacgaacgttgtgccgggaggccgcaagctgtgcgccatcaccaaggtaggactagaccaccaggcgctgctcggcgggacgctggcggtggttgcgcgcgagaaggcggggattgtggtgccaggcgtgcggtttgtggcggtggacggaacgaacgcgccgagtgtgcttgcggaggtgcgtgctgcggcagcgaaggtgggggcggaggtgcacgagacgggaggggcgcctgtgtgtacggtttcatggggcgcagtggcggcgagcgcgctgccgctggcgggtgcgtaccaagtacagaacgcaggcgtggcgctagcgctgctagaccatctgcagcaactgggggagatctcggtgagtcacgcggcactggagcgcgggctcaaagcagtggagtggccgggacggctgcagcaggtggagtacgacctgggcggcgtccacgtcccactgctctttgacggggcgcacaacccctgtgctgcagaggagctggctcgctttctcaatgagaggtaccgcgggcccggaggctctccgctcatttacgtactggcggtgacatgtggcaaagagatcgatgcgcttctggcgccgctgttaaagccgcacgaccgagtgttcgccacatcgttcggagccgtggagagcatgccgtgggtagcggctatggcctccgaggacgttgcggcagcggctcgtcgctacacggcgcacgtgtctgcggtagcggacccgctggacgccctgcgggcggcagctgccgcccggggcgacgcaaaccttgtcgtttgcggctcgctttatcttgtgggcgagctgctgcgccgcgagcactag

SEQ ID NO: 59 - DNA, 20 bp, Artificial Sequence
  Primer
taggaggcgagagcacaaga

SEQ ID NO: 60 - DNA, 20 bp, Artificial Sequence
  Primer
gccgagttcctttgtgatgc

SEQ ID NO: 61 - DNA, 20 bp, Artificial Sequence
  Primer
gcccgagaacagcggattta

SEQ ID NO: 62 - DNA, 20 bp, Artificial Sequence
  Primer
cgcaagaacaaacaggcgtt

SEQ ID NO: 63 - DNA, 20 bp, Artificial Sequence
  Primer
tggcgttatggttgtcgttg

SEQ ID NO: 64 - DNA, 20 bp, Artificial Sequence
  Primer
taaacacgcctctgactgct

SEQ ID NO: 65 - DNA, 20 bp, Artificial Sequence
  Primer
ggcggagcgcaattatacac

SEQ ID NO: 66 - DNA, 20 bp, Artificial Sequence
  Primer
caggaaagtgtctgtcgcct

SEQ ID NO: 67 - DNA, 20 bp, Artificial Sequence
  Primer
gattggccgcttacacatgg

SEQ ID NO: 68 - DNA, 20 bp, Artificial Sequence
  Primer
aacgtttgggcttctaccga

SEQ ID NO: 69 - DNA, 20 bp, Artificial Sequence
  Primer
cagctcgtgtcgtgagatgt

SEQ ID NO: 70 - DNA, 20 bp, Artificial Sequence
  Primer
agagtgcccaactgaatgct

SEQ ID NO: 71 - DNA, 21 bp, Artificial Sequence
  Primer
gccctgcataaggaatttaac

SEQ ID NO: 72 - DNA, 23 bp, Artificial Sequence
  Primer
agcttatggacatacgactgatg

SEQ ID NO: 73 - DNA, 20 bp, Artificial Sequence
  Primer
tgaacggacagacatggtcg

SEQ ID NO: 74 - DNA, 20 bp, Artificial Sequence
  Primer
acagcgtgagccgtatgatt

SEQ ID NO: 75 - DNA, 53 bp, Bacillus subtilis
ctgtatccgcgggtgaaagagagtgttttacatataaaggaggagaaacaatg

SEQ ID NO: 76 - DNA, 53 bp, Artificial Sequence
  Regulatory region containing mutation
ctgtatccgcgggtgaaagagagtgttttacatataaaagaggagaaacaatg

SEQ ID NO: 77 - DNA, 2742 bp, Artificial Sequence
  BS-FOLOP1-COMB
gcgcgctacgtagggcgcgctagcggccaattttgtcaaaataattttattgacaacgtcttattaacgttgatataatttaaattttatttgacaaaaatgggctcgtgttgtacaataaatgtatattaagaggaggagatatatataatgatttatgtcaacggaagatacatggaagaaaaagacgcagtcctgagcccgtttgatcatggctttctttatggaattggagtttttgaaacgttccgcctttatgaaggatgtccgtttctgcttgattggcatatcgaacgccttgaaagagctctgaaagaccttcaaatcgaatatacagttagcaaacatgaaattcttgaaatgcttgataaacttcttaaacttaatgatattaaagatggcaacgcgcgcgtcagacttaatatctcagcaggaattagcgataaaggctttgtcgcgcagacgtatgataaacctacggttctttgcttcgttaatcaacttaaaccggaaagccttccgcttcaaaaagaaggcaaagtccttagcatcagacgcaatacgcctgaaggatcatttagactgaaatctcatcactaccttaataatatgtatgcgaaacgcgaaattggcaatgatccgcgcgtcgaaggaatctttctgacggaggatggcgcagttgcagaaggaatcatttctaacgttttttggcgcaaaggccgctgcatttatacaccgtcacttgatacaggcatcctggatggagtcacacgcagatttatcattgaaaatgcaaaagatattggccttgaacttaaaacaggacgctatgaacttgaagcgctgcttacggcggatgaagcgtggatgacgaactcagtccttgaaattattccgtttacaaaaatcgaagaagtcaactatggctcacagagcggagaagcaacgtcagcgcttcaacttctttacaaaaaagaaataaagaacatgattcatgaaaagggaggaagagcatggcgcagcacacaatagatcaaacacaagtcattcatacgaaaccgagcgcgctttcatataaagaaaaaacactggtcatgggcattcttaacgttacacctgattcttttagcgatggtggaaaatatgacagcttggacaaggcgcttctgcatgccaaagaaatgatcgacgacggcgcgcacattattgacataggaggcgagagcacaagaccgggagctgaatgcgtcagcgaagacgaagaaatgtctcgggtcattccggtcattgaacgcatcacaaaggaactcggcgtcccgatttcagtggatacatataaagcatctgtggcagacgaagcagtcaaagcgggcgcatctattatcaatgacatttggggagcgaaacatgatccgaagatggcaagcgtcgcagcggaacataacgttccaattgtcctgatgcacaatcggccagaacggaattataacgaccttcttccggatatgctgagcgaccttatggaatcagtcaaaattgcggttgaggcgggcgtggatgagaaaaatattattttagatccgggcatcggcttcgcgaagacataccatgataatcttgcagtgatgaataagttagagatcttcagcggacttggctatcctgtcctgctggctacatctcgtaaaagatttatcggaagagttcttgatttaccgcctgaagagagagcagagggcacaggagcgacagtctgcttgggcattcagaaaggatgcgacatagtgcgtgttcatgatgtcaagcaaattgccagaatggcgaaaatgatggacgcgatgctgaataagggaggggtgcaccatggataaagtttatgtggaaggaatggaattttatggctatcatggcgtcttcacagaagagaacaaattgggacaacgcttcaaagtagatctgacagcagaactggatttatcaaaagcaggacaaacagacgaccttgaacagacaattaattatgcagagctttaccatgtctgtaaagacattgtcgaaggcgagccggtcaaattggtagagacccttgctgagcggatagctggcacagttttaggtaaatttcagccggttcaacaatgtacggtgaaagttattaaaccagatccgccgattcctggccactataaatcagtagcaattgaaattacgagaaaaaagtcatgaacaacattgcgtacattgcgcttggctctaatattggagatagagaaacgtatctgcgccaggccgttgcgttactgcatcaacatgctgcggtcacagttacaaaagtcagctcaatttatgaaacagatccggtcggctatgaagaccaagcccagtttttaaatatggcggttgaaattaaaacaagcctgaatccgtttgaacttctggaactgacacagcaaatcgaaaacgaactgggccgcacacgcgaagttagatggggcccgagaacagcggatttagacattctgctgtttaacagagaaaacattgaaacagagcagttaattgtcccgcatcctcgcatgtatgaacgcctgtttgttcttgcgccgcttgcggaaatttgccagcaggtcgagaaagaagcgacaagcgcggaaacggatcaagaaggagttagagtttggaaacaaaaatcaggcgttgatgaatttgtacatagcgaaagctaagaattcaattgttgcg

SEQ ID NO: 78 - DNA, 1239 bp, Artificial Sequence
  BS-FOLOP2-COMB
gaattcaattttgtcaaaataattttattgacaacgtcttattaacgttgatataatttaaattttatttgacaaaaatgggctcgtgttgtacaataaatgttactagagtaaaggaggaaactagtatgaaagaagtcaataaagaacaaattgaacaggcagtgagacagattcttgaagcaattggagaagatccgaacagagagggcttacttgatacaccgaaaagagttgctaaaatgtatgcggaagtcttttcaggcttaaatgaagatccgaaagagcattttcagacaattttcggagaaaaccatgaagagctggtccttgtgaaagatattgcgtttcactcaatgtgcgaacatcacctggtgccgttttacggcaaggcacacgttgcgtatattcctagaggcggaaaagttacaggcttgtcaaaattagcacgcgcagttgaagctgttgcaaaaagaccgcaattacaggaacgcattacatctacaattgcggaatcaattgtcgagacattagaccctcatggcgttatggttgtcgttgaagctgaacacatgtgcatgacaatgcgcggcgtcagaaaacctggcgcaaaaacagtcacatcagcagtcagaggcgtgtttaaagatgatgcggcagctcgtgcggaagtcctggaacatattaaacgccaggactaataaagatagagggtgctataaatgatttcatttattttcgcaatggacgcgaatagactgataggcaaagacaatgatctgccgtggcatttaccgaatgacctggcttattttaaaaaaattacaagcggccatagcatcattatgggacgtaaaacatttgagtcaattggcagacctcttccgaacagaaaaaacattgttgtcacatctgcgccggattcagaatttcagggctgcacagtcgtctcaagccttaaagacgttcttgatatttgcagcggaccggaagagtgttttgtcattggcggagcgcaattatacacagatctttttccgtacgcggatagactgtatatgacaaaaatccaccatgaatttgaaggcgacagacactttcctgaatttgacgagagcaactggaaactcgtgtctagcgaacagggcacgaaggatgagaaaaatccgtatgactatgaatttcttatgtatgaaaagaaaaacagcagcaaagcgggaggcttttaactcgagtttt

SEQ ID NO: 79 - protein, 415 aa, Bacillus subtilis
MKHLPAQDEQVFNAIKNERERQQTKIELIASENFVSEAVMEAQGSVLTNKYAEGYPGKRYYGGCEHVDVVEDIARDRAKEIFGAEHVNVQPHSGAQANMAVYFTILEQGDTVLGMNLSHGGHLTHGSPVNFSGVQYNFVEYGVDKETQYIDYDDVREKALAHKPKLIVAGASAYPRTIDFKKFREIADEVGAYFMVDMAHIAGLVAAGLHPNPVPYADFVTTTTHKTLRGPRGGMILCREEFGKKIDKSIFPGIQGGPLMHVIAAKAVSFGEVLQDDFKTYAQNVISNAKRLAEALTKEGIQLVSGGTDNHLILVDLRSLGLTGKVAEHVLDEIGITSNKNAIPYDPEKPFVTSGIRLGTAAVTSRGFDGDALEEVGAIIALALKNHEDEGKLEEARQRVAALTDKFPLYKELDY

SEQ ID NO: 80 - DNA, 1248 bp, Bacillus subtilis
atgaaacatttacctgcgcaagacgaacaagtgtttaacgccattaaaaatgagcgtgaacgccaacagactaagatcgaattgattgcttctgagaactttgtaagtgaagcggttatggaagcacaaggatctgttttgacaaataagtacgcagaaggatatccgggcaaacgctactacggcggatgcgagcacgtcgatgtcgttgaagatatcgcccgtgaccgcgcgaaggaaatctttggagcggagcatgtaaacgttcagcctcattcaggcgcacaagcaaacatggcagtgtacttcacgattttggagcaaggcgatactgtacttgggatgaacctctcccacggcggccatttaacacacggaagcccagtcaacttcagcggtgttcaatataactttgttgagtacggcgtagataaagaaactcaatatattgattacgatgacgtgcgtgaaaaagccctcgctcataaaccgaagcttatcgtagcaggagcaagtgcgtatcctcgtacaatcgactttaagaaattccgtgaaattgctgatgaagtcggcgcttacttcatggtggatatggcacatatcgcaggacttgttgcggcaggccttcatccaaacccggttccttacgctgatttcgttacaacaacaacacataaaacacttcgcggtcctcgcggcggtatgatcctttgccgtgaagagtttggcaagaaaattgataaatcgatcttccctggaattcaaggcggccctctgatgcacgttattgccgcaaaagctgtttcattcggtgaagtattgcaggacgatttcaaaacatatgcacaaaacgtcatttcaaacgcgaaacgtctggctgaagccttaacgaaagagggcatccagctcgtttcaggcggaacagacaaccaccttatccttgttgaccttcgttcgctcggactgactggtaaggttgcggagcatgtacttgatgaaattggtattacgtctaacaaaaacgcgattccatatgatcctgaaaaacctttcgtaacaagcggcatccgtcttggtacagctgctgtaaccagccgcggttttgacggagacgcattagaagaagtcggtgctatcattgcgcttgcattgaaaaaccacgaagatgaaggaaaacttgaagaagcaagacagcgtgtagctgctctgactgataaatttcctttatataaagaattagattattaa

SEQ ID NO: 81 - protein, 300 aa, Bacillus subtilis
MKSYMTQRLDEYRDGNEDKGRLLVSCPDQPGIVSAVSAFLFEHGANIIESNQYTTDPEGGRFFLRIEFDCAGIREKKSSLQAAFASVAEKFDMTWSLTLASELKRVAIFVSKELHCLHELIWEWQTGNLMAEIAVVISNHEEARELVERLNIPFHYMKANKDIRAEVEKKQLELLEQYDVDVIVLARYMQILTPDFVSAHPNRIINIHHSFLPAFIGANPYKRAYERGVKLIGATSHYVTNDLDEGPIIEQDIERVDHRDNAEALKNIGRTIERSVLARAVKWHLEDRVIVHENKTIVFN

SEQ ID NO: 82 - DNA, 903 bp, Bacillus subtilis
atgaaatcatatatgactcagcggttggacgaataccgtgacggaaatgaggataaaggtcggctcttggtcagctgccccgatcagccgggtatcgtctctgcagtttccgcgtttttatttgaacacggtgccaatattatagaatcaaatcaatatacgacagaccctgaaggcggccggttcttcctgagaatcgaattcgactgcgcaggcattcgtgaaaaaaaatcatcacttcaggcagcgtttgcctctgttgcggaaaaattcgacatgacatggagcttaactttggcgagcgaactgaagcgtgtcgccattttcgtttcaaaggagctccactgcctgcatgagctgatttgggaatggcaaaccggcaacctgatggcggagatcgctgttgtcatcagtaaccatgaggaagcgagagagctggttgagcgcctgaacattccattccactatatgaaagcgaacaaagacatcagagcggaagtcgaaaagaagcagcttgaactgctggagcagtacgatgttgatgtgatcgtgctcgcacgctatatgcagattctaactcctgattttgtttcggctcatccgaatcgcatcatcaatatccaccattcattcctgccagcttttatcggtgcgaatccgtacaaacgggcctacgagcgcggcgtgaaactgatcggtgcgacatctcattatgtgacaaacgatcttgatgaagggccgatcattgaacaggatattgagcgtgtggaccaccgcgataatgcggaagcgctgaaaaacatcggaagaacaattgagcgcagcgtgcttgcccgtgctgtgaaatggcatttggaagaccgtgtcatcgttcatgaaaataaaacaatcgtctttaactag

SEQ ID NO: 83 - protein, 612 aa, Bacillus subtilis
MGLLEDLQRQVLIGDGAMGTLLYSYGIDRCFEELNISKPEEIQRIHKAYVEAGANIIQTNTYGANYIKLSRHGLEDDIKKMNQEAVKIARASAGDAYVLGTMGGIRTFNKNAYSLDEIKRSFREQLYLLLHEEPDGLLLETYYDLEEAREVLKIARKETDLPIMLNVSMHEQGVLQDGTPLSDALRSIADLGADIVGINCRLGPYHMIEALSEVPIFDDVFLSVYPNSSLPSLEEGRLVYETDDTYFQNSASEFRKQGARIIGGCCGTTPNHIRAMAEAVGGLAPITEKEVKTRAKEFISVHHERTEPGLDEIAAKKRSIIVELDPPKKLSFDKFLSAAAELKEAGIDALTLADNSLATPRISNVACGALVKQQLDMRSLVHITCRDRNIIGLQSHLMGLDTLGLNDVLAITGDPSKIGDFPGATSVYDLTSFDLIRLIKQFNEGLSLSGKPLGKKTNFSVAAAFNPNVRHLDKAVKRLEKKIDCGADYFVSQPVYSEQQLVDIHNETKHLKTPIYIGIMPLTSSRNAEFIHNEIPGIKLSDTIREKMAHAGEDKEKQKAEGLAIARSLLDTACELFNGIYLITPFLRSDLTAELTSYIQQKDEQRQNIFLH

SEQ ID NO: 84 - protein, 296 aa, Escherichia coli
MSFFHASQRDALNQSLAEVQGQINVSFEFFPPRTSEMEQTLWNSIDRLSSLKPKFVSVTYGANSGERDRTHSIIKGIKDRTGLEAAPHLTCIDATPDELRTIARDYWNNGIRHIVALRGDLPPGSGKPEMYASDLVTLLKEVADFDISVAAYPEVHPEAKSAQADLLNLKRKVDAGANRAITQFFFDVESYLRFRDRCVSAGIDVEIIPGILPVSNFKQAKKFADMTNVRIPAWMAQMFDGLDDDAETRKLVGANIAMDMVKILSREGVKDFHFYTLNRAEMSYAICHTLGVRPGL

SEQ ID NO: 85 - DNA, 1839 bp, Bacillus subtilis
atgggacttttagaagatttgcaaagacaggtgttaatcggtgacggcgccatggggacgctcctctactcctatggcattgacaggtgttttgaggagctcaatatttcaaagccggaggaaattcagcgcatacataaagcgtacgttgaagcgggagccaacattattcaaacgaatacgtacggagccaactatattaaattatccagacacgggcttgaggatgacatcaaaaaaatgaatcaggaggccgtaaagatcgcgcgggcttcagcaggcgatgcatacgtgcttggaacgatgggcggcatacgcacatttaataaaaacgcatacagtcttgacgaaattaagcgcagcttccgcgagcagctgtatctgctgctgcatgaagagccggatggcttgctgttagaaacatattatgatttggaagaagcccgtgaagtgctgaaaatcgcgcgcaaagagactgatcttccgattatgctcaatgtctccatgcacgagcaaggcgtattgcaggacggaacgccgctttctgacgcattgcgctcgattgcggatctcggagctgatattgtcggcattaactgccgcctcggcccttatcacatgattgaagcgctaagcgaagtgccgatttttgatgacgtattcttatccgtttatccgaacagcagtcttccttcgcttgaagagggacggctcgtctatgaaacagacgacacgtattttcaaaacagcgcgtcagaattccgtaagcaaggtgcgcggattatcggcggctgctgcggcacaacgccgaatcacatcagggcgatggcggaagcggttggcgggctggctccgattacggaaaaagaagtcaaaacccgggcgaaagaattcatttctgttcatcacgagcgaaccgagcccggcctggatgagatcgcagcgaaaaaacgctctattatcgtagagctggacccgccgaaaaaactaagctttgacaaatttctgtccgctgctgctgaattaaaagaagcgggaatcgatgccctgacactcgccgacaattctttggcgaccccgcggatcagcaatgttgcctgcggcgcccttgtcaagcagcagctcgatatgcgttcccttgtccatattacgtgccgtgaccgtaacattatcggcttacagtcacatttaatgggccttgatacgttagggctgaatgacgtgcttgctattacaggtgacccttctaaaatcggggattttcctggggcgacgtctgtatatgatttaacatcttttgatttaatcaggctgatcaaacaattcaatgaaggtctgtctctgtctggaaaaccgctcggcaagaaaacgaatttctcagttgccgccgcatttaatcctaacgtccgccatctggataaagcggtaaaacggcttgagaaaaaaatagattgcggcgcagattattttgtttctcagcctgtctattccgagcagcagcttgtcgatatccataacgaaacaaaacatctgaagacgccgatctatatcggcatcatgccgctgacaagcagccggaatgccgaattcatccataatgaaattcccggcattaagctgtctgatacgattcgcgaaaaaatggcccatgccggtgaagataaagaaaagcaaaaggcggaaggccttgcaattgcacgctccctgctggatacggcatgcgagctgtttaacggcatctacttgataacgcctttcctcagatctgacttaaccgctgagctcacatcatatattcagcaaaaagatgagcagcgccaaaatatctttttgcattaa

SEQ ID NO: 86 - DNA, 891 bp, Escherichia coli
atgagcttttttcacgccagccagcgggatgccctgaatcagagcctggcagaagtccaggggcagattaacgtttcgttcgagtttttcccgccgcgtaccagtgaaatggagcagaccctgtggaactccatcgatcgccttagcagcctgaaaccgaagtttgtatcggtgacctatggcgcgaactccggcgagcgcgaccgtacgcacagcattattaaaggcattaaagatcgcactggtctggaagcggcaccgcatcttacttgcattgatgcgacgcccgacgagctgcgcaccattgcacgcgactactggaataacggtattcgtcatatcgtggcgctgcgtggcgatctgccgccgggaagtggtaagccagaaatgtatgcttctgacctggtgacgctgttaaaagaagtggcagatttcgatatctccgtggcggcgtatccggaagttcacccggaagcaaaaagcgctcaggcggatttgcttaatctgaaacgcaaagtggatgccggagccaaccgcgcgattactcagttcttcttcgatgtcgaaagctacctgcgttttcgtgaccgctgtgtatcggcgggcattgatgtggaaattattccgggaattttgccggtatctaactttaaacaggcgaagaaatttgccgatatgaccaacgtgcgtattccggcgtggatggcgcaaatgttcgacggtctggatgatgatgccgaaacccgcaaactggttggcgcgaatattgccatggatatggtgaagattttaagccgtgaaggagtgaaagatttccacttctatacgcttaaccgtgctgaaatgagttacgcgatttgccatacgctgggggttcgacctggtttataa

SEQ ID NO: 87 - DNA, 19 bp, Artificial Sequence
  Primer
gggcgcgctagcggccaat

SEQ ID NO: 88 - DNA, 75 bp, Artificial Sequence
  Primer
acgttgtcaataaaattattttgacaaaattgaattcttagctttcgctatgtacaaattcatcaacgcctgatt

SEQ ID NO: 89 - DNA, 75 bp, Artificial Sequence
  Primer
aatcaggcgttgatgaatttgtacatagcgaaagctaagaattcaattttgtcaaaataattttattgacaacgt

SEQ ID NO: 90 - DNA, 20 bp, Artificial Sequence
  Primer
gcggtagtggtgcttacgat

SEQ ID NO: 91 - DNA, 1248 bp, Artificial Sequence
  Optimized glyA sequence for Bacillus subtilis codon usage
atgaagcaccttcctgcacaagatgaacaagtattcaacgctattaagaacgaacgcgaacgtcaacaaaccaagattgaattgatcgctagcgaaaatttcgtttcagaagcagttatggaggctcaaggctctgttcttacgaacaaatatgctgaaggctacccaggcaaaagatactacggaggctgcgaacacgttgacgtagtcgaagacattgcacgtgaccgcgcaaaagaaatcttcggcgcagaacatgttaacgttcaacctcacagcggagcacaagcgaacatggcagtttattttacaatccttgaacaaggggacacagtcctgggcatgaacctttctcacggaggccatcttactcatggtagcccggtgaacttttctggcgtccaatataacttcgttgagtacggtgtagataaagaaacacagtatattgactatgatgatgtgcgtgaaaaagctcttgcgcacaaacctaaacttattgttgcgggagcgtcagcgtacccgcgtaccattgattttaaaaagttccgggaaattgctgacgaggttggcgcgtactttatggtggatatggctcatattgcgggcttggtcgcagcaggactgcaccctaacccggtgccttacgccgatttcgttacaaccacgacacataaaacacttagaggcccacgtggcggaatgattctttgcagagaagaatttggaaagaaaattgacaaatccattttccctggcatccagggaggtcctcttatgcatgtcattgcagcaaaagcggtgagcttcggcgaagtgttacaagatgatttcaaaacgtatgctcagaacgttatctctaatgcgaaacgtttagcggaagcgttgacaaaagaaggtatccaacttgtctcaggaggcacagataatcatcttattctggttgatcttcgctcacttggattaacaggcaaagttgcggaacatgttcttgacgaaattggtattacatcaaacaaaaacgctattccttacgatccggaaaaacctttcgtaacatctggtattcgcttaggcacagctgcggttacaagccgcggattcgatggcgatgcgcttgaagaggtaggcgctattattgcactggcgttaaaaaatcatgaggatgaaggaaaacttgaagaggcccgccagcgtgtcgcagctcttactgataaattcccgctttacaaagaacttgattattga

SEQ ID NO: 92 - DNA, 903 bp, Artificial Sequence
  optimized purU sequence for Bacillus subtilis codon usage
atgaagtcttacatgactcaacgtttagatgaatatcgtgacggcaacgaggataaaggacgcctcttggtgtcttgtccggaccagcctggaattgtttctgcagtttccgcatttctttttgagcacggcgcaaatatcatcgaaagcaatcaatacacaactgacccagaaggcggccgtttcttccttcgcattgaatttgactgcgcaggaatccgtgaaaaaaaaagcagcttacaggctgctttcgcatctgtcgctgaaaaattcgacatgacttggtctttaactttggcgtctgagctaaaacgtgttgctatctttgtgtcaaaagagctccactgcttacatgaacttatttgggaatggcaaacaggtaacctaatggcagagattgccgtagtcatctcaaaccacgaagaagctcgcgaactggtggaacgtcttaatatcccttttcactacatgaaagctaacaaagacatccgggctgaagttgaaaaaaaacagcttgagctgttagaacagtatgacgttgacgtaatcgtgttagcacgctatatgcagattttaacaccggacttcgtctctgctcatcctaaccgtattatcaatattcaccattctttcttaccggcctttatcggagcaaatccttataaacgtgcgtatgaacgtggagttaaattgatcggcgccacgtcccactatgttacgaacgacttagatgaaggtcctattatcgaacaagatatcgagcgtgttgatcatcgtgataatgctgaagcactcaaaaatattggccgtacaatcgagcgctctgttctagctcgtgcagtaaaatggcatcttgaggaccgcgtcatcgttcatgagaataaaacaatcgtttttaactga

SEQ ID NO: 93 - DNA, 1839 bp, Artificial Sequence
  optimized yitJ sequence for Bacillus subtilis codon usage
atgggattacttgaagatcttcaacgccaagttctgatcggagacggagcgatgggaacgcttctgtattcttacggaatcgacagatgttttgaagagttaaatatttctaaacctgaagaaatccaaagaatccataaagcgtacgtcgaagcaggagcaaacatcattcaaacaaatacatacggagctaactacattaaacttagccgccacggcctggaagacgacattaaaaaaatgaaccaagaagcggtcaaaattgcacgcgcaagcgctggcgacgcgtatgttcttggcacgatgggaggcatccgcacattcaacaagaatgcttacagcctggacgagattaaacgctcattccgtgaacaattatacctgcttcttcacgaagaaccggatggccttcttttagaaacatattacgatctggaagaggcacgtgaggttcttaaaattgctcgcaaagagacggatcttccgatcatgcttaacgtctctatgcacgaacaaggcgtgcttcaggatggcacacctcttagcgacgctttaagaagcatcgcggacctgggcgcagatatcgttggcattaactgccgcctgggaccgtaccacatgattgaagcattgtcagaagtcccgatcttcgatgacgtcttccttagcgtttacccgaactcttcattaccgagcttagaagaaggacgcttagtgtatgaaacggacgatacatacttccaaaattctgcaagcgagttccgtaaacaaggagcgcgtattatcggcgggtgctgcggaacaactccgaatcacattagagcgatggcagaagcagtaggaggtcttgcacctatcacagaaaaagaagtgaaaacgcgtgctaaagaatttatttctgttcatcacgaacgtacagaaccgggcttagacgaaattgctgctaaaaagcgcagcattattgtcgaacttgatccgcctaaaaaactttcatttgataaatttctttcagcggcagcggagttaaaagaagcaggcatcgatgctcttacattagcggataactcattagcgactcctcgtatctctaacgtcgcatgcggtgcacttgtgaaacaacaactggatatgcgctcacttgttcatatcacgtgccgtgaccgcaatatcattggccttcaatctcatttaatgggtcttgatacattaggtttaaacgatgtacttgcgattacaggtgatccgtcaaaaattggtgactttcctggcgcaacatcagtgtacgacctgacctcttttgatcttattcgtcttattaaacaattcaatgaaggattatctttgtctggtaaacctcttggcaagaaaacgaacttctcagtagcggcggcattcaatcctaatgtgcgtcaccttgacaaagcggtcaaaagacttgaaaaaaaaatcgactgtggagcggattatttcgtctctcaaccagtatatagtgagcaacaattagtggatatccacaacgaaacgaagcacttaaaaacaccaatctacattggaatcatgcctcttacgtcctctcgcaacgctgaatttatccataacgaaattccaggaattaaactttctgatacgatccgtgaaaaaatggcccatgctggcgaagacaaggagaaacaaaaggctgaaggacttgcaattgcgcgttctttactggacactgcttgcgaattgttcaacggcatctatcttattacaccatttcttcgtagcgacttgacagccgagttgacatcatatattcaacaaaaggacgaacaacgccagaatatttttctgcattaa

SEQ ID NO: 94 - DNA, 891 bp, Artificial Sequence
  DNA fragment
atgtcattcttccatgcttcacaacgtgatgcgcttaaccagagcctggcagaagttcagggccaaatcaatgtgtctttcgaattttttcctcctcgcacaagcgagatggaacaaacactttggaactccatcgaccgtctttcatcacttaagccgaaatttgtctcagtgacgtacggcgctaacagcggagaacgcgatagaacacattcaatcattaagggaatcaaagatcgtacgggattagaagctgctcctcacttaacatgtattgatgcgacgccggatgaacttcgtacaatcgcgcgtgattactggaacaatggtattcgtcatatcgtcgctctgagaggagatttgcctcctggatctggtaaaccggagatgtacgcttcagaccttgtaacacttcttaaagaagtggcggactttgatatctctgtcgcggcttaccctgaagttcatccggaggcaaaaagcgcgcaggcagacttattaaaccttaagcgtaaagtagacgcgggtgctaaccgtgcgatcacacagttcttttttgatgtggaaagttatttacgttttcgcgatcgttgcgtttcggcaggcatcgatgttgaaatcatccctggaattttaccagttagcaacttcaaacaggcgaaaaaatttgcggatatgacgaacgttcgtattccggcatggatggcgcaaatgtttgacggacttgacgatgacgcagaaacacgcaaattagtcggtgctaacattgcgatggacatggttaaaattctgtcacgcgaaggtgtcaaggactttcatttctacacacttaatcgcgctgaaatgagctatgcgatctgccacacacttggagtccgtcctggcctttaa

SEQ ID NO: 95 - DNA, 1686 bp, Bacillus subtilis
cttgttgttgttccgccgttttacggcagtcctttatcggtgacggggacaattgtgcttgtaccggagccggaacccgtctacgcgtttacagcgaatccaaatgatcaatcagtttctgtgattgatacaaacaccgatactgttgtgacaacgattgctcttccgtacaatccggcaggtattgaaattacgccagataaaagcgctgtgtttgtcttacatcccaacaataatgtgatttctgtcatcgattatgacacattaacagtgacagcaactatattgctggatcagccgcctcgattgattagatttatccctaatcatgagtttgcttatgttttcaccggtactgcggtttatgtgattggaattgatacgttaactgtggataggtcgattcctgtggaaggatatgatgttgcaatcgatcccaatgggttattcgcctatgttctgaattttggaatagtgcaaaaagtggacctaactaccggtgaagtcacaggaacaattgaacgagagcttatcgtatcaaccatagaaacaaattggccggagcggtatgcctatgtattagaacaagaattcttttttaattatttgacggttattgatttaaatacgtttaccatcagcagcacccaagagctggagtatgaaggggaatatcgaatgtttacgagtggagcagaggtgtatttatatgacggcttcactggcaatttatattctgtcagtccaaatggagcaggcgttataggaaatgttccgcaatcagcaacagactatgcgtttaccccgaatggcgattttctgtatgcaactcgttttatagagcagagcatcattgtttacaacacagatgattattctgaggaaactgtgatatctcttggggtttcaccgggtgccattacgatttaaccgctgcatgttgtgcatttctaaaaggacgtgaaatcatgaaaaagaatgatctttctgctttacaggaaaattgtttctgtttttgtgatgaggaaattagccgggaagcgccgtttcaagtgccgatagatttccctgagggcttcaaggttgatacggccgaagcgtcagcggctgttacatggagtacagacaatttgtcctgtatcagtgagccttgcttaatccagactgggccggaaccagaagatatcggtgttcgatatgctgtgagagtacaagggaccattacgcttcttgtcagtgtgtcgcctgtacgaaatcaatatggacaaggagacggggctgtttctgtgattcatactgaagagattgatcaagttgtttattatgcagcacagtctggccgctgtcctgatttcagtcaaataacagtggaagatctgctcattgtaccgcctttctacggcagtccgcttacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagttggcttcgttgggattctccggtgatgctgatttcgctgc

SEQ ID NO: 96 - DNA, 2126 bp, Artificial Sequence
  spectinomycin selectable marker
cggtagtcattgctggcgaacgcgtctagagctccggcataaaattaactaaaataattattatgtagataaaaaatttagaagccaatgaaatctataaataaactaaattaagtttatttaattaacaactatggatataaaataggtactaatcaaaatagtgaggcgaatatatttgaatacatacgaacaaatcaataaagtgaaaaaaatacttcggaaacatttaaaaaataaccttattggtacttacatgtttggatcaggagttgagagtggactcaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaacttatagataattatcaggatgatgaaaccaactctatattaactttatgccgcatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatcttacgataaactatcttaataaccgccttaagaaactttaacccgggcgcgttgctttcgatgaagttttaccgccatcaccagaccctcaatcgtatgtgtttactgccaatactggagacagcacggtttcggttattgatgcagatttaaacactgttgtaaaaacaatccctttttctgacgtgccaactaatctaggcgttactttcgataaagcattcacgtatgtcctccatggaaataccaaccttgtctcagtgattgataataaaacattaaccatcataaataccattactgttggaggagggccgagaaaaatcgaatttgatccaacagatgagttcgcatacgtgatggccgccggatctatatatgtgattaacacggcctctcaatcggtcattgatgtcattccgatacccggcgctttagactttgctcttgatccgaatggacagtatgtctataccgctaatggaagcagttggtcggttgataaatatgatgtaaatactgggcagttggtagaaagtatcatagatacatttgaatttcccagtttaattacaactccttacgctggaaattttgcgtacgtattgaatggtgagctgtggccgaaaggtgtaacggagattagtttgtcaccattaagcaggggaggggacttcagccgtttatttgaaacgctgaggacgattgtattttctctcgatagtactagagcttattttcaagagccatatagtgaaccgtttctcatcaataacttatacgtagttaatacggccagacagaggatcattgcaaacgtgtctctccccggcgcctttgacctcgccgtaacgccggacaaacagtatatttatgcggcacagcccaacgacaatgctgtgaccgtgtaccgcacaagtgattatacagccgtgacggtgattccggttggcgcgggtccgtctgcgattgcgatgtagctattttatagatcaagagaagcggtgctgtgataagagaatcgtttataaataaatgatccgatttgctgaatggcagccaggataatcagaaacagcagttcacttatagagaaactgagcatccaccaaatcccggctgctgccagtgctcccgcaaacacttttctgaacaggctgccgtgccggaaaagctgccgcaaatacgttcgcatgcaaaacaataaatacaggatcagtagcatcgcaagtccca

SEQ ID NO: 97 - DNA, 7709 bp, Artificial Sequence
  MTHF-OP-A operon
cgcgtttacagcgaatccaaatgatcaatcagtttctgtgattgatacaaacaccgatactgttgtgacaacgattgctcttccgtacaatccggcaggtattgaaattacgccagataaaagcgctgtgtttgtcttacatcccaacaataatgtgatttctgtcatcgattatgacacattaacagtgacagcaactatattgctggatcagccgcctcgattgattagatttatccctaatcatgagtttgcttatgttttcaccggtactgcggtttatgtgattggaattgatacgttaactgtggataggtcgattcctgtggaaggatatgatgttgcaatcgatcccaatgggttattcgcctatgttctgaattttggaatagtgcaaaaagtggacctaactaccggtgaagtcacaggaacaattgaacgagagcttatcgtatcaaccatagaaacaaattggccggagcggtatgcctatgtattagaacaagaattcttttttaattatttgacggttattgatttaaatacgtttaccatcagcagcacccaagagctggagtatgaaggggaatatcgaatgtttacgagtggagcagaggtgtatttatatgacggcttcactggcaatttatattctgtcagtccaaatggagcaggcgttataggaaatgttccgcaatcagcaacagactatgcgtttaccccgaatggcgattttctgtatgcaactcgttttatagagcagagcatcattgtttacaacacagatgattattctgaggaaactgtgatatctcttggggtttcaccgggtgccattacgatttaaccgctgcatgttgtgcatttctaaaaggacgtgaaatcatgaaaaagaatgatctttctgctttacaggaaaattgtttctgtttttgtgatgaggaaattagccgggaagcgccgtttcaagtgccgatagatttccctgagggcttcaaggttgatacggccgaagcgtcagcggctgttacatggagtacagacaatttgtcctgtatcagtgagccttgcttaatccagactgggccggaaccagaagatatcggtgttcgatatgctgtgagagtacaagggaccattacgcttcttgtcagtgtgtcgcctgtacgaaatcaatatggacaaggagacggggctgtttctgtgattcatactgaagagattgatcaagttgtttattatgcagcacagtctggccgctgtcctgatttcagtcaaataacagtggaagatctgctcattgtaccgcctttctacggcagtccgcttacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgaagcaccttcctgcacaagatgaacaagtattcaacgctattaagaacgaacgcgaacgtcaacaaaccaagattgaattgatcgctagcgaaaatttcgtttcagaagcagttatggaggctcaaggctctgttcttacgaacaaatatgctgaaggctacccaggcaaaagatactacggaggctgcgaacacgttgacgtagtcgaagacattgcacgtgaccgcgcaaaagaaatcttcggcgcagaacatgttaacgttcaacctcacagcggagcacaagcgaacatggcagtttattttacaatccttgaacaaggggacacagtcctgggcatgaacctttctcacggaggccatcttactcatggtagcccggtgaacttttctggcgtccaatataacttcgttgagtacggtgtagataaagaaacacagtatattgactatgatgatgtgcgtgaaaaagctcttgcgcacaaacctaaacttattgttgcgggagcgtcagcgtacccgcgtaccattgattttaaaaagttccgggaaattgctgacgaggttggcgcgtactttatggtggatatggctcatattgcgggcttggtcgcagcaggactgcaccctaacccggtgccttacgccgatttcgttacaaccacgacacataaaacacttagaggcccacgtggcggaatgattctttgcagagaagaatttggaaagaaaattgacaaatccattttccctggcatccagggaggtcctcttatgcatgtcattgcagcaaaagcggtgagcttcggcgaagtgttacaagatgatttcaaaacgtatgctcagaacgttatctctaatgcgaaacgtttagcggaagcgttgacaaaagaaggtatccaacttgtctcaggaggcacagataatcatcttattctggttgatcttcgctcacttggattaacaggcaaagttgcggaacatgttcttgacgaaattggtattacatcaaacaaaaacgctattccttacgatccggaaaaacctttcgtaacatctggtattcgcttaggcacagctgcggttacaagccgcggattcgatggcgatgcgcttgaagaggtaggcgctattattgcactggcgttaaaaaatcatgaggatgaaggaaaacttgaagaggcccgccagcgtgtcgcagctcttactgataaattcccgctttacaaagaacttgattattgaaaaagaggggagggaaacattatgatgaagtcttacatgactcaacgtttagatgaatatcgtgacggcaacgaggataaaggacgcctcttggtgtcttgtccggaccagcctggaattgtttctgcagtttccgcatttctttttgagcacggcgcaaatatcatcgaaagcaatcaatacacaactgacccagaaggcggccgtttcttccttcgcattgaatttgactgcgcaggaatccgtgaaaaaaaaagcagcttacaggctgctttcgcatctgtcgctgaaaaattcgacatgacttggtctttaactttggcgtctgagctaaaacgtgttgctatctttgtgtcaaaagagctccactgcttacatgaacttatttgggaatggcaaacaggtaacctaatggcagagattgccgtagtcatctcaaaccacgaagaagctcgcgaactggtggaacgtcttaatatcccttttcactacatgaaagctaacaaagacatccgggctgaagttgaaaaaaaacagcttgagctgttagaacagtatgacgttgacgtaatcgtgttagcacgctatatgcagattttaacaccggacttcgtctctgctcatcctaaccgtattatcaatattcaccattctttcttaccggcctttatcggagcaaatccttataaacgtgcgtatgaacgtggagttaaattgatcggcgccacgtcccactatgttacgaacgacttagatgaaggtcctattatcgaacaagatatcgagcgtgttgatcatcgtgataatgctgaagcactcaaaaatattggccgtacaatcgagcgctctgttctagctcgtgcagtaaaatggcatcttgaggaccgcgtcatcgttcatgagaataaaacaatcgtttttaactgaaaaagaggggagggaaacattatgatgggattacttgaagatcttcaacgccaagttctgatcggagacggagcgatgggaacgcttctgtattcttacggaatcgacagatgttttgaagagttaaatatttctaaacctgaagaaatccaaagaatccataaagcgtacgtcgaagcaggagcaaacatcattcaaacaaatacatacggagctaactacattaaacttagccgccacggcctggaagacgacattaaaaaaatgaaccaagaagcggtcaaaattgcacgcgcaagcgctggcgacgcgtatgttcttggcacgatgggaggcatccgcacattcaacaagaatgcttacagcctggacgagattaaacgctcattccgtgaacaattatacctgcttcttcacgaagaaccggatggccttcttttagaaacatattacgatctggaagaggcacgtgaggttcttaaaattgctcgcaaagagacggatcttccgatcatgcttaacgtctctatgcacgaacaaggcgtgcttcaggatggcacacctcttagcgacgctttaagaagcatcgcggacctgggcgcagatatcgttggcattaactgccgcctgggaccgtaccacatgattgaagcattgtcagaagtcccgatcttcgatgacgtcttccttagcgtttacccgaactcttcattaccgagcttagaagaaggacgcttagtgtatgaaacggacgatacatacttccaaaattctgcaagcgagttccgtaaacaaggagcgcgtattatcggcgggtgctgcggaacaactccgaatcacattagagcgatggcagaagcagtaggaggtcttgcacctatcacagaaaaagaagtgaaaacgcgtgctaaagaatttatttctgttcatcacgaacgtacagaaccgggcttagacgaaattgctgctaaaaagcgcagcattattgtcgaacttgatccgcctaaaaaactttcatttgataaatttctttcagcggcagcggagttaaaagaagcaggcatcgatgctcttacattagcggataactcattagcgactcctcgtatctctaacgtcgcatgcggtgcacttgtgaaacaacaactggatatgcgctcacttgttcatatcacgtgccgtgaccgcaatatcattggccttcaatctcatttaatgggtcttgatacattaggtttaaacgatgtacttgcgattacaggtgatccgtcaaaaattggtgactttcctggcgcaacatcagtgtacgacctgacctcttttgatcttattcgtcttattaaacaattcaatgaaggattatctttgtctggtaaacctcttggcaagaaaacgaacttctcagtagcggcggcattcaatcctaatgtgcgtcaccttgacaaagcggtcaaaagacttgaaaaaaaaatcgactgtggagcggattatttcgtctctcaaccagtatatagtgagcaacaattagtggatatccacaacgaaacgaagcacttaaaaacaccaatctacattggaatcatgcctcttacgtcctctcgcaacgctgaatttatccataacgaaattccaggaattaaactttctgatacgatccgtgaaaaaatggcccatgctggcgaagacaaggagaaacaaaaggctgaaggacttgcaattgcgcgttctttactggacactgcttgcgaattgttcaacggcatctatcttattacaccatttcttcgtagcgacttgacagccgagttgacatcatatattcaacaaaaggacgaacaacgccagaatatttttctgcattaaattaattctagagctccggcataaaattaactaaaataattattatgtagataaaaaatttagaagccaatgaaatctataaataaactaaattaagtttatttaattaacaactatggatataaaataggtactaatcaaaatagtgaggcgaatatatttgaatacatacgaacaaatcaataaagtgaaaaaaatacttcggaaacatttaaaaaataaccttattggtacttacatgtttggatcaggagttgagagtggactcaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaacttatagataattatcaggatgatgaaaccaactctatattaactttatgccgcatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatcttacgataaactatcttaataaccgccttaagaaactttaacccgggcgcgttgctttcgatgaagttttaccgccatcaccagaccctcaatcgtatgtgtttactgccaatactggagacagcacggtttcggttattgatgcagatttaaacactgttgtaaaaacaatccctttttctgacgtgccaactaatctaggcgttactttcgataaagcattcacgtatgtcctccatggaaataccaaccttgtctcagtgattgataataaaacattaaccatcataaataccattactgttggaggagggccgagaaaaatcgaatttgatccaacagatgagttcgcatacgtgatggccgccggatctatatatgtgattaacacggcctctcaatcggtcattgatgtcattccgatacccggcgctttagactttgctcttgatccgaatggacagtatgtctataccgctaatggaagcagttggtcggttgataaatatgatgtaaatactgggcagttggtagaaagtatcatagatacatttgaatttcccagtttaattacaactccttacgctggaaattttgcgtacgtattgaatggtgagctgtggccgaaaggtgtaacggagattagtttgtcaccattaagcaggggaggggacttcagccgtttatttgaaacgctgaggacgattgtattttctctcgatagtactagagcttattttcaagagccatatagtgaaccgtttctcatcaataacttatacgtagttaatacggccagacagaggatcattgcaaacgtgtctctccccggcgcctttgacctcgccgtaacgccggacaaacagtatatttatgcggcacagcccaacgacaatgctgtgaccgtgtaccgcacaagtgattatacagccgtgacggtgattccggttggcgcgggtccgtctgcgattgcgatgtagctattttatagatcaagagaagcggtgctgtgataagagaatcgtttataaataaatgatccgatttgctgaatggcagccaggataatcagaaacagcagttcacttatagagaaactgagcatccaccaaatcccggctgctgccagtgctcccgcaaacacttttctgaacaggctgccgtgccggaaaagctgccgcaaatacgttcgcatgcaaaacaataaatacaggatcagtagcatcgcaagtccca

SEQ ID NO: 98 - DNA, 7709 bp, Artificial Sequence; identical to SEQ ID NO: 97
  MTHF-OP-B operon

SEQ ID NO: 99 - DNA, 1260 bp, Lactobacillus reuteri
gtgcggacctatgaacaaattaacgcgggctttaatcgccaaatgctgggaggacaacgggatcgggtaaaatttttgcgacgaattcttactcgtttaggaaaccctgaccagcgatttaaaattatccatattgcagggacaaacggaaaaggctcgactgggacaatgttagaacaggggttacaaaatgctggctaccgggtcggctattttagcagtcctgctttggtagatgatcgggagcaaattaaggttaatgaccatttgattagtaaaaaagattttgcgatgacttaccaaaagatcaccgaacatttacccgctgatcttttacctgatgatattaccatttttgagtggtggaccctgataatgctgcaatattttgcagaccaaaaagtggattgggcagtcattgaatgcggtttaggcggtcaagatgatgcaaccaatattatcagcgcgccttttattagtgtgataacgcatattgcccttgaccatacacggattttgggtccaacgattgccaaaattgcccaggctaaagcaggaattatcaagaccggtactaaacaagtgtttttagcgccgcaccaagaaaaagacgctctcacaattattagagaaaaggctcaacagcaaaaagtaggattaacgcaagcagatgcccagtcaattgttgatggaaaggcgatcctgaaagttaatcataaaatttataaagttccttttaatttattagggacatttcaaagtgaaaatttgggcaccgttgtcagtgtatttaattttctttatcaacgaagacttgttacttcttggcagcctttattatcgacgcttgcgacagtgaagattgctggtcgaatgcagaaaatcgctgatcatccccctattattttggacggggcccataacccagatgcagccaagcaacttaccaaaactatttctaagcttcctcataataaagtaattatggttcttggctttctcgctgataaaaatattagtcaaatggttaaaatttatcagcaaatggcagatgagataataattactactcctgaccatccaactcgcgcccttgatgcaagtgcgttaaaatcagttctgcctcaagcgattattgccaacaatccccgacaagggttagtggtggcaaaaaagattgcggagccgaatgatttaattattgtaacgggttctttttatacgattaaagatatcgaggccaatttagatgaaaaataa

SEQ ID NO: 100 - protein, 762 aa, Bacillus subtilis
MTTIKTSNLGFPRIGLNREWKKALEAYWKGSTDKDTFLKQIDELFLSAVKTQIDQQIDVVPVSDFTQYDHVLDTAVSFNWIPKRFRHLTDATDTYFAIARGIKDAVSSEMTKWFNTNYHYIVPEYDESIEFRLTRNKQLEDYRRIKQEYGVETKPVIVGPYTFVTLAKGYEPSEAKAIQKRLVPLYVQLLKELEEEGVKWVQIDEPALVTASSEDVRGAKELFESITSELSSLNVLLQTYFDSVDAYEELISYPVQGIGLDFVHDKGRNLEQLKTHGFPTDKVLAAGVIDGRNIWKADLEERLDAVLDILSIAKVDELWIQPSSSLLHVPVAKHPDEHLEKDLLNGLSYAKEKLAELTALKEGLVSGKAAISEEIQQAKADIQALKQFATGANSEQKKELEQLTDKDFKRPIPFEERLALQNESLGLPLLPTTTIGSFPQSAEVRSARQKWRKAEWSDEQYQNFINAETKRWIDIQEELELDVLVHGEFERTDMVEYFGEKLAGFAFTKYAWVQSYGSRCVRPPVIYGDVEFIEPMTVKDTVYAQSLTSKHVKGMLTGPVTILNWSFPRNDISRKEIAFQIGLALRKEVKALEDAGIQIIQVDEPALREGLPLKTRDWDEYLTWAAEAFRLTTSSVKNETQIHTHMCYSNFEDIVDTINDLDADVITIEHSRSHGGFLDYLKNHPYLKGLGLGVYDIHSPRVPSTEEMYNIIVDALAVCPTDRFWVNPDCGLKTRQQEETVAALKNMVEAAKQARAQQTQLV

SEQ ID NO: 101 - DNA, 2289 bp, Bacillus subtilis
atgacaaccatcaaaacatcgaatttaggatttccgagaatcggactgaaccgggaatggaaaaaagcacttgaagcgtattggaaaggcagtactgataaagatacgtttttgaagcaaatcgacgaactatttttatccgcagtaaaaacacaaattgaccagcagattgatgttgtgcctgtttctgatttcacacagtatgaccatgtactcgacacagcagtcagcttcaactggatcccgaaacggttcagacatttgactgacgctaccgatacatacttcgctatcgcccgcggaatcaaagacgctgtatctagtgaaatgacaaaatggtttaatacaaattaccattacatcgttccggaatatgacgagagcattgagttccgtctgacaagaaacaaacaactcgaagattatcgccggatcaaacaggaatacggtgtggaaacaaagcctgtgattgtcggcccttatacgttcgttacgcttgctaaaggctatgaaccgtctgaagcaaaagcgatccaaaaacgccttgtgccattatatgtacagcttttgaaagagcttgaagaagaaggcgtaaaatgggttcaaatcgatgagccggcgctcgttaccgcctctagtgaagatgtacgcggcgcaaaagaattatttgaaagcattacaagtgagctttcatccttgaatgtgcttttgcagacgtattttgattctgttgatgcttatgaagagctgatctcttacccggttcagggaattggccttgatttcgttcacgacaaaggcagaaacctggaacagcttaaaacacatggcttcccgacagataaagtgctggcagccggcgttatcgacggacgcaacatttggaaagcggaccttgaagagcgtctcgatgccgttcttgatattctcagcattgcaaaagttgatgaactgtggattcagccttccagcagcctgctgcatgttccagtagcgaaacaccctgatgagcatttggaaaaagacctattgaacggattatcctacgcaaaagaaaagctggccgagctgacagctttgaaagaaggcttagtatcaggaaaagcggcgatcagcgaagagattcagcaggctaaggctgatatccaggcgcttaaacagtttgcaacaggcgccaattctgaacaaaagaaagagcttgagcaattaactgataaagacttcaagcgcccgattccgtttgaagaacgtttagccctacaaaatgaatctctcggccttccgcttttgccgacgacaacgatcggcagcttcccgcagtctgctgaagtgcggagcgcacgccaaaaatggcggaaagctgagtggtccgatgaacagtatcaaaactttatcaatgcggaaacaaaaagatggattgatattcaggaagaattggagcttgatgtccttgttcacggcgaatttgaacggacagacatggtcgaatacttcggtgaaaagctggccggtttcgccttcactaaatatgcctgggttcaatcatacggctcacgctgtgtccgcccgccagtcatttacggagatgttgaatttattgaaccgatgacagtgaaagacacagtctacgcacagtcattgacttccaagcatgtgaaaggaatgctgacgggcccggttacaatcttaaactggtctttccctcgaaacgacatctcgaggaaagaaatcgccttccaaatcgggcttgcccttcgcaaagaagttaaagcgcttgaagacgcaggcattcaaatcattcaagtcgatgaaccagcgctgcgtgaaggccttccattgaaaacccgcgattgggatgagtatttgacttgggcggcagaagctttcagattaaccacttcttccgtgaaaaacgagacacaaattcatacacatatgtgctacagcaacttcgaagatatcgttgatacaatcaatgatcttgatgccgatgtgattacaatcgaacatagcagaagccacggaggatttttagattacttaaaaaaccacccgtatttgaaagggcttggccttggtgtatatgacattcacagccctcgtgtgccgtcaactgaagaaatgtacaatattatcgttgatgcgcttgccgtctgtccgactgaccgcttctgggtaaatccagactgcggattgaaaacaagacagcaggaagaaacggttgcagcattgaaaaatatggttgaagccgcaaaacaggcaagagcacagcagacacagctagtataa